(19) United States
(12) Patent Application Publication
(10) Pub. No.: US 2025/0250637 A1
(43) Pub. Date: Aug. 7, 2025

(54) METHODS AND SYSTEMS FOR IMMUNOME PROFILING FOR CANCER DIAGNOSIS AND TREATMENT PROGNOSIS

(71) Applicants: Krishnan VENKATAKRISHNAN, Toronto (CA); Bo TAN, Toronto (CA)

(72) Inventors: Krishnan VENKATAKRISHNAN, Toronto (CA); Bo TAN, Toronto (CA); Swarna GANESH, Toronto (CA); Rupa RAVINDRA Haldavnekar, Toronto (CA)

(21) Appl. No.: 18/856,112

(22) PCT Filed: Apr. 12, 2023
(86) PCT No.: PCT/CA2023/050501

§ 371 Date: Oct. 11, 2024

Related U.S. Application Data
(60) Provisional application No. 63/330,123, filed on Apr. 12, 2022.

(51) Int. Cl. C12Q 1/6886; C12N 5/09; G01N 33/543; G01N 33/574

(52) U.S. Cl. CPC C12Q 1/6886; C12N 5/0693; G01N 33/54346; G01N 33/54373; G01N 33/574; C12N 2502/30; C12Q 2600/112; C12Q 2600/118; C12Q 2600/154

(57) ABSTRACT

Provided is a method of providing a cancer assessment for a subject, comprising (i) measuring a value of one or more cancer traits of at least one tumor associated immune cell (TAIC) or TAIC-derived biomarker in a fluid sample obtained from the subject to obtain a test profile; (ii) comparing the test profile to one or more reference profiles; (iii) wherein similarity or a differential in the test profile to the one or more reference profiles is indicative of one or more cancer characteristics; and (iv) based on the one or more cancer characteristics, providing the cancer assessment of the subject.

### RELATED APPLICATION

This application claims the benefit of priority to U.S. Provisional Application No. 63/330,123, filed Apr. 12, 2022, the entire contents of which is hereby incorporated by reference in its entirety.

### FIELD

This disclosure relates generally to cancer detection and prognosis, and more specifically to methods and apparatus for early cancer detection and prognosis using tumor associated immune cells as biomarkers.

### BACKGROUND

The cancer burden has continued to grow globally with there being an estimated 18 million cancer cases and 9.6 million deaths caused by cancer in 2018. The majority of cancers can be cured when the disease gets diagnosed while it is still confined to the organ of origin. Therefore, rapid screening of onset of cancer is a key to curing cancer.

Unfortunately, many types of cancers only get diagnosed at advanced stages because the diagnostic intervention is often related to symptoms. Therefore, cancer screening, a strategy for early cancer intervention, needs to be employed to detect disease markers before the presentation of noticeable symptoms.

Early detection and monitoring recurrence have shown to have potential possibilities toward reducing cancer mortality. So far, research on non-invasive cancer diagnosis relies on endogenous biomarkers such as circulating cell free DNA, circulating tumor cells and exosomes. Cancer diagnosis using endogenous biomarkers are limited in sensitivity due to the rapid biomarker clearance rate, low concentration in circulation and high background signals, since these biomarkers are present in healthy individuals as well. Hence, there is need to focus on the immune system, which characterizes the first line of defense against the abnormal changes to homeostasis such as cancer.

Studies have shown that immune responses are associated with tumor growth by altering the tumor microenvironment and are well characterized with cancer progression and survival (Kresovich, J. K. et al., 2020). Immune profiles obtained after cancer diagnosis may be associated with prognosis but whether the immune profile can be used as a diagnostic marker remains largely unexplored.

### SUMMARY

In accordance with one broad aspect, at least one example embodiment described in accordance with the teachings herein provides a method of determining one or more cancer characteristics of a subject, the method comprising:
measuring a value of one or more cancer traits of at least one tumor associated immune cell (TAIC) or TAIC-derived biomarker in a fluid sample obtained from the subject to obtain a test profile;
comparing the test profile to one or more reference profiles;
wherein similarity or a differential in the test profile to the one or more reference profiles is indicative of one or more cancer characteristics.

In one embodiment, the method further comprises providing a cancer assessment of the subject based on the one or more cancer characteristics.

In one embodiment, the cancer characteristic is a presence or absence of cancer, a cancer type, a cancer stage, a cancer grade, a primary cancer, a metastatic cancer, cancer potential for metastasis, a molecular phenotype of the cancer, a benign cancer, a malignant cancer, the tissue of origin of the cancer, a metastatic location of the cancer or a combination thereof.

In another embodiment, the at least one tumor associated immune cell comprises a T cell, a NK cell, a macrophage, a dendritic cell, a B cell, or a neutrophil, or a combination thereof.

In another embodiment, the one or more TAIC-derived biomarkers comprise metabolic changes, activation status or a surface phenotype of the at least one tumor associated immune cell.

In another embodiment, the one or more TAIC-derived biomarkers comprise one or more extracellular vesicles from the at least one tumor associated immune cell.

In another embodiment, the one or more TAIC-derived biomarkers comprise cell-free nucleic acid of the at least one tumor associated immune cell.

In another embodiment, the cell-free nucleic acid is cell-free DNA, miRNA, lncRNA, mRNA or histone-associated DNA.

In another embodiment, the cell-free DNA is molecularly modified by one or more of methylation, oxidation, phosphorylation and acetylation.

In another embodiment, the one or more TAIC-derived biomarkers comprise the structure and molecular composition of the cell-free DNA.

In another embodiment, the one or more TAIC-derived biomarkers comprise DNA methylation status of the at least one tumor associated immune cell.

In another embodiment, the one or more cancer traits is measured by Raman spectroscopy.

In another embodiment, the one or more cancer traits is measured by DNA Next gen sequencing; PCR; repertoire sequencing; immunosequencing; single cell sequencing; single cell RNA sequencing; unique molecular identifier (UMI); optical microscopy techniques, optionally confocal microscopy, immunohistochemistry, super resolution microscopy; flow cytometry; or mass cytometry.

In another embodiment, the reference profile is derived from one or more reference TAICs.

In another embodiment, the reference TAIC is a T cell, a NK cell, a macrophage, dendritic cell, B cells or a neutrophil.

In another embodiment, the reference TAIC is a cancer cell associated T cell, a cancer stem cell associated T cell, or a tumor associated stem cell like T cell.

In another embodiment, the reference TAIC is a cancer cell associated NK cell, a cancer stem cell associated NK cell, or a tumor associated stem cell like NK cell.

In another embodiment, the reference TAIC is a cancer cell associated macrophage, a cancer stem cell associated macrophage, or a tumor associated stem cell like macrophage.

In another embodiment, the reference TAIC is a cancer cell associated dendritic cell, a cancer stem cell associated dendritic cell, or a tumor associated stem cell like dendritic cell.

In another embodiment, the reference TAIC is a cancer cell associated B cell, a cancer stem cell associated B cell, or a tumor associated stem cell like B cell.

In another embodiment, the reference TAIC is a cancer cell associated neutrophil, a cancer stem cell associated neutrophil, or a tumor associated stem cell like neutrophil.

In another embodiment, the fluid is blood plasma, serum, urine, stool, mucus or cerebrospinal fluid.

In another embodiment, providing the cancer assessment comprises providing a cancer type, a cancer location, a stage of the cancer, a metastatic potential of the cancer, or a cancer therapy efficacy.

In another embodiment, the providing the cancer assessment comprises providing a prognosis for the patient, early cancer diagnosis, determining whether a tumor is benign or malignant, determining whether a tumor is primary or metastatic, determining whether a primary tumor has potential for metastasis, determining a progression of the cancer, determining a nodal metastasis of the cancer, determining a clinical metastasis of the cancer, predicting patient survival, providing a prognosis for the patient, providing an early diagnosis of cancer, includes providing the early diagnosis of hard to detect cancers, determining a presence of an aggressive brain cancer, providing a location of the tumor, monitoring cancer recurrence during or after therapy, and/or determining a presence of minimal residual disease.

In another embodiment, the method further comprises when the tumor is benign, determining whether the tumor has potential for malignancy.

In another embodiment, the cancer is brain cancer, optionally glioblastoma, astrocytoma, or oligodendroglioma.

In another embodiment, the cancer is ovarian cancer, brain cancer, bladder cancer, breast cancer, colon cancer, esophageal cancer, gastric cancer, hepatic cancer, intestinal cancer, lung cancer, heat and neck cancer, rectal cancer, prostate cancer, pancreatic cancer, thyroid cancer, cervical cancer, melanoma, nasopharyngeal cancer, testicular cancer, or uterine cancer.

In accordance with another broad aspect, at least one example embodiment described in accordance with the teachings herein provides a method of determining one or more cancer characteristics for a subject, the method comprising:
isolating a volume of a fluid from a fluid sample of the subject, the volume of fluid including at least one tumor associated immune cell (TAIC) or TAIC-derived biomarker;
adding at least a portion of the volume of fluid to a nanosensor, the nanosensor comprising nanoparticles configured to capture the at least one TAIC) or TAIC-derived biomarker and amplify signals emitted by the at least one TAIC or TAIC-derived biomarker during Raman spectroscopy;
performing Raman spectroscopy on the volume of fluid on the nanosensor to produce a sample Raman spectrum, the sample Raman spectrum having amplified signals indicating the presence of the at least one TAIC or TAIC-derived biomarker on the nanosensor;
processing the sample Raman spectrum using data from template Raman spectra having cancer characteristics to detect whether the sample comprises one or more of the cancer characteristics.

In one embodiment, the method further comprises providing a cancer assessment of the subject based on the detected one or more cancer characteristics.

In one embodiment, the one or more cancer characteristics of the sample are detected based on determining which correlation values obtained by correlating the amplified signals of the sample Raman spectrum to template Raman spectra from the known cancer samples having the cancer characteristics are larger than a correlation threshold.

In another embodiment, the one or more cancer characteristics of the sample are detected by:
performing feature extraction on the Raman sample spectral data to extract feature values;
performing classification by applying the feature values to at least one set of classification models determined for the at least one TAIC or TAIC-derived biomarker to detect the one or more cancer characteristics; and
providing the cancer assessment by incorporating each of the detected cancer characteristics,
wherein the classification models are determined using the template Raman spectra from the known cancer samples.

In another embodiment, the feature extraction is performed using Principal Component Analysis, Multivariate Curve Resolution Analysis or a combination thereof.

In another embodiment, the classification model comprises Partial Least Squares Discriminant Analysis (PLSDA), Support Vector Machine Discriminant Analysis (SVMDA) and Artificial Neural Network analysis (ANN) tSNE and Random Forest classification.

In another embodiment, the sample Raman spectrum includes second amplified signals indicating a presence of a second biomarker on the nanosensor and the method further comprises:
performing further data processing on the sample Raman spectrum to compare the second amplified signals to a second template Raman spectrum to determine a correlation between the sample Raman spectrum and the second template Raman spectrum, the template Raman spectrum being of a known cancer characteristic, and
based on both the correlation between the sample Raman spectrum and the template Raman spectrum and the second correlation between the sample Raman spectrum and the second template Raman spectrum, providing a diagnosis of the cancer in the patient.

In accordance with another broad aspect, at least one example embodiment described in accordance with the teachings herein provides a method of generating a reference tumor associated immune cell (TAIC) for use in cancer assessment, the method comprising coculturing one or more naïve immune cells with a tumor and/or cancer cell line until the one or more reference tumor associated immune cell (TAICs) expresses one or more cancer traits.

In one embodiment, the one or more naïve immune cell comprises a T cell, an NK cell, a macrophage, a dendritic cell, a B cell, a neutrophil, or a combination thereof.

In another embodiment, the cancer assessment includes providing a type of the cancer, a location of the cancer, a stage of the cancer, a grade of the cancer, a metastatic potential of the cancer, or a therapy efficacy of the cancer.

In accordance with another broad aspect, at least one example embodiment described in accordance with the teachings herein provides a computing device for providing a cancer assessment for a subject or for determining one or more cancer characteristics for a subject.

In at least one embodiment, the processing unit is further configured to perform any of the steps of the methods described in accordance with the teachings herein.

These and other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a flow chart (a) showcasing the workflow for discovering cancer-specific signatures in immune cells derived from patients diagnosed with cancer, and a potential workflow (b) followed in a clinical setting for cancer diagnosis using the disclosed methods.

FIG. 2 shows an assessment of the viability of tumor associated immune cells for comprehensive cancer assessment. A) Similarity analysis validating the individual immune cells signature in the buffy coat profile from cancer patient. b) Principal component analysis showing the tumor-associated immune signals overlap between genders (top). Bar graphs of the individual macromolecular peak positions show no statistical significance (bottom). c) Principal component analysis showing the tumor-associated immune signals overlap between different the age groups below 45 years and above 75 years (top). Bar graphs of the individual macromolecular peak positions show no statistical significance (bottom).

FIG. 3 shows the discovery of a unique T cell signature specific to the presence of cancer. a) t-distributed stochastic neighbor embedding (tSNE) plot showcasing the differences in T cell phenotype of tumor-associated T cells and non-tumor T cells. b) Violin plot showing that T cells in early-stage cancer show distinct differences in phenotype compared to non-cancer. c) tSNE plot showing that T cells in early-stage cancer show distinct differences in phenotype compared to late-stage cancer. d) Heat map (bottom) and dendrogram (top) showcasing the distinct differences are contributed by peaks represented in PC1.

FIG. 4 shows an application of the unique tumor-associated T cell signature for cancer diagnosis, and for early cancer diagnosis. a) Scatter plot showcasing the probability of a sample to be diagnosed as cancer based on tumor-associated T cell signature. b) Calibration curve showing the performance of machine learning model for diagnosis of cancer using the tumor-associated T cell signature, with a sensitivity and specificity each of 100%. c) Principal components contributing to accurate cancer diagnosis. D) ROC curve for cancer prediction. e) Scatter plot showcasing the probability of a sample to be diagnosed as early-stage cancer based on tumor-associated T cell signature. f) Calibration curve showing the performance of machine learning model for diagnosis of early-stage cancer using tumor-associated T cell signature, with a sensitivity and specificity each of 100%. g) Principal components contributing to accurate early-stage cancer diagnosis. h) ROC curve for early-stage cancer prediction, with an AUC of 1.00.

FIG. 5 shows an application of the unique tumor-associated T cell signature for cancer staging and for diagnosis of metastasis. a) Scatter plot showcasing the probability of a sample to be for cancer staging based on tumor-associated T cell signatures. b) Calibration curve showing the performance of machine learning model for cancer staging using tumor-associated T cell signature, with a sensitivity of 98.4% and a specificity of 100%. c) Principal components contributing to accurate cancer staging. d) ROC curve for cancer staging, showing an AUC of 1.00. e) Scatter plot showcasing the probability that a sample is metastatic based on tumor-associated T cell signature. f) Calibration curve showing the performance of machine learning model to detect metastasis using tumor-associated T cell signature, with a sensitivity of 96.2% and a specificity of 100%. g) Principal components contributing to accurate detection of metastasis. h) ROC curve for prediction of cancer metastasis, showing an AUC of 1.00.

FIG. 6 shows a schematic representation of methods for using circulating natural killer cell profiling (CNKP) with the OncoImmune Probe Platform for cancer diagnosis. SERS training data are obtained from cancer cell associated NK cells and cancer stem cell (CSC) associated NK cells generated in vitro (left, top panel), and from healthy NK cells (left, bottom panel). Middle: The three NK cell phenotypes form the basis for cancer diagnosis using a simple machine learning paradigm. Once the principal components responsible for distinguishing cancer associated NK cells from healthy NK cells are identified from the training data, the model can make cancer predictions using SERS data obtained from patient blood samples (right panel).

FIG. 7 shows the discovery of a unique NK cell signature specific to the presence of cancer. a) tSNE plot showcasing the differences in NK cell phenotype of tumor-associated NK cell and non-tumor NK cell b) NK cells in early-stage cancer show distinct differences in phenotype compared to non-cancer. c) NK cells in early-stage cancer show distinct differences in phenotype compared to late-stage cancer. d) Dendrogram (left) and heat map (right) showcasing that the distinct differences are contributed by peaks represented in PC1.

FIG. 8 shows an application of the unique tumor-associated NK cell signature for cancer diagnosis and for early cancer diagnosis. a) Scatter plot showcasing the probability of a sample to be diagnosed as cancer based on tumor-associated NK cell signature b) Calibration curve showing the performance of machine learning model for diagnosis of cancer using tumor-associated NK cell signature, with a sensitivity and specificity each of 100%. C) Principal components contributing to accurate cancer diagnosis. d) ROC curve for cancer prediction, with an AUC of 1.0. e) Scatter plot showcasing the probability of a sample to be diagnosed as early-stage cancer based on tumor-associated NK cell signature. f) Calibration curve showing the performance of machine learning model for diagnosis of early-stage cancer using tumor-associated NK cell signature, with a sensitivity and specificity each of 100%. g) Principal components contributing to accurate early-stage cancer diagnosis. h) ROC curve for early-stage cancer prediction, with an AUC of 1.00.

FIG. 9 shows an application of the unique tumor-associated NK cell signature for cancer staging and for diagnosis of cancer metastasis. a) Scatter plot showcasing the probability of a sample to be for cancer staging based on tumor-associated NK cell signature. b) Calibration curve showing the performance of machine learning model for cancer staging using tumor-associated NK cell signature, with a sensitivity and specificity each of 100%. c) Principal components contributing to accurate cancer staging. d) ROC curve for cancer staging, with an AUC of 1.00. e) Scatter plot showcasing the probability that a sample is metastatic based on tumor-associated NK cell signature. f) Calibration curve showing the performance of machine learning model to detect metastasis using tumor-associated NK cell signature, with a sensitivity of 99.6% and a specificity of 100%. g) Principal components contributing to accurate detection of metastasis. h) ROC curve for prediction of cancer metastasis, with an AUC of 1.00.

FIG. 10 shows the discovery of a unique macrophage signature specific to the presence of cancer. a) tSNE plot showcasing the differences in macrophage phenotype of tumor-associated macrophage and non-tumor macrophage. b) Macrophages in early-stage cancer show distinct differences in phenotype compared to non-cancer. c) Macrophages in early-stage cancer show distinct differences in phenotype compared to late-stage cancer. d) Dendrogram (left) and heat map (right) showcasing the distinct differences are contributed by peaks represented in PC1.

FIG. 11 shows an application of the unique tumor-associated macrophage signature for cancer diagnosis and for early cancer diagnosis. a) Scatter plot showcasing the probability of a sample to be diagnosed as cancer based on tumor-associated macrophage signature. b) Calibration curve showing the performance of machine learning model for diagnosis of cancer using tumor-associated macrophage signature, with a sensitivity and specificity each of 100%. c) Principal components contributing to accurate cancer diagnosis. d) ROC curve for cancer prediction, with an AUC of 1.00. e) Scatter plot showcasing the probability of a sample to be diagnosed as early-stage cancer based on tumor-associated macrophage signature. f) Calibration curve showing the performance of machine learning model for diagnosis of early-stage cancer using tumor-associated macrophage signature, with a sensitivity of 98.5% and a specificity of 100%. g) Principal components contributing to accurate early-stage cancer diagnosis. h) ROC curve for early-stage cancer prediction, with an AUC of 1.00.

FIG. 12 shows an application of the unique tumor-associated macrophage signature for cancer staging and for diagnosis of cancer metastasis. a) Scatter plot showcasing the probability of a sample to be for cancer staging based on tumor-associated macrophage signature. b) Calibration curve showing the performance of machine learning model for cancer staging using tumor-associated macrophage signature, with a specificity and sensitivity each of 100%. c) Principal components contributing to accurate cancer staging. d) ROC curve for cancer staging, with an AUC of 1.00. e) Scatter plot showcasing the probability of metastasis based on tumor-associated macrophage signature. f) Calibration curve showing the performance of machine learning model to detect metastasis using tumor-associated macrophage signature, with a specificity and sensitivity each of 100%. g) Principal components contributing to accurate detection of metastasis. h) ROC curve for prediction of cancer metastasis, with an AUC of 1.00.

FIG. 13 shows the discovery of a unique dendritic cell signature specific to the presence of cancer. a) tSNE plot showcasing the differences in dendritic cell phenotype of tumor-associated dendritic cells and non-tumor dendritic cells. b) Dendritic cells in early-stage cancer show distinct differences in phenotype compared to non-cancer. c) Dendritic cells in early-stage cancer show distinct differences in phenotype compared to late-stage cancer. D) Dendrogram (left) and heat map (right) showcasing the distinct differences are contributed by peaks represented in PC1

FIG. 14 shows an application of the unique tumor-associated dendritic cell signature for cancer diagnosis and for early cancer diagnosis. a) Scatter plot showcasing the probability of a sample to be diagnosed as cancer based on tumor-associated dendritic cell signature. b) Calibration curve showing the performance of machine learning model for diagnosis of cancer using tumor-associated dendritic cell signature, with a specificity and sensitivity each of 100%. c) Principal components contributing to accurate cancer diagnosis d) ROC curve for cancer prediction. e) Scatter plot showcasing the probability of a sample to be diagnosed as early-stage cancer based on tumor-associated dendritic cell signature. f) Calibration curve showing the performance of machine learning model for diagnosis of early-stage cancer using tumor-associated dendritic cell signature, with a sensitivity of 97.1% and a specificity of 96.7% g) Principal components contributing to accurate early-stage cancer diagnosis. h) ROC curve for early-stage cancer prediction, with an AUC of 0.998.

FIG. 15 shows the discovery of a unique B cell signature specific to the presence of cancer. A) tSNE plot showcasing the differences in B cell phenotype of tumor-associated B cells and non-tumor B cells. b) B cell in early-stage cancer show distinct differences in phenotype compared to non-cancer. c) B cell in early-stage cancer show distinct differences in phenotype compared to late-stage cancer. d) Dendrogram (left) and heat map (right) showcasing the distinct differences are contributed by peaks represented in PC1.

FIG. 16 shows an application of the unique tumor-associated B cell signature for cancer diagnosis and for early cancer diagnosis. a) Scatter plot showcasing the probability of a sample to be diagnosed as cancer based on tumor-associated B cell signature. b) Calibration curve showing the performance of machine learning model for diagnosis of cancer using tumor-associated B cell signature, with a sensitivity and specificity each of 100%. c) Principal components contributing to accurate cancer diagnosis. d) ROC curve for cancer prediction. e) Scatter plot showcasing the probability of a sample to be diagnosed as early-stage cancer based on tumor-associated B cell signature. f) Calibration curve showing the performance of machine learning model for diagnosis of early-stage cancer using tumor-associated B cell signature, with a sensitivity and specificity each of 100%. g) Principal components contributing to accurate early-stage cancer diagnosis. h) ROC curve for early-stage cancer prediction, with an AUC of 1.00.

FIG. 17 shows an application of the unique signature of tumor-associated immune cells for multi-cancer early diagnosis and tumor tissue of origin determination in cancers from breast, lung, colorectal, renal, uterine, testicular, and brain (including astrocytoma, oligodendroglioma, glioblastoma), ovarian, head and neck tissues. a) Tumor-associated T cells for multicancer early diagnosis. b) Tumor-associated macrophages for multicancer early diagnosis. c) Tumor-associated dendritic cells for multicancer early diagnosis. d) Tumor-associated NK cells for multicancer early diagnosis. e) Accuracy of multicancer early diagnosis using tumor-associated immune cells, nearing 100%. f) Specificity of multicancer early diagnosis using tumor-associated immune cells, nearing 100%. g) Sensitivity of multicancer early diagnosis using tumor-associated immune cells, nearing 100%.

FIG. 18 shows the application of the unique signature of tumor-associated immune cells for diagnosis of early-stage tumors based on size. a) Scatter plot showcasing the probability of a sample to be diagnosed as cancer based on tumor-associated immune cell signature. b) Calibration curve showing the performance of machine learning model for diagnosis of cancer using tumor-associated immune cell signature, with a sensitivity of 100% and a specificity of 98.3%. c) Principal components contributing to accurate cancer diagnosis. d) ROC curve for cancer prediction. e) Multicancer diagnosis correctly diagnosing between 5 cancers at early stage based on tumor sizes of 0.5 to 2.4 cm.

FIG. 19 shows multi cancer diagnosis using the combined signature of tumor associated immune cells including T cells, NK cells, B cells, dendritic cells and macrophages. Specificity, sensitivity and accuracy of the model were each 100%.

FIG. 20 shows a schematic of neutrophil interaction in the tumor microenvironment (TME) of ovarian cancer (OC). The top panel depicts schematics of healthy and cancerous ovaries. Neutrophils are observed to interact with ovarian cancer cells and ovarian cancer stem cells in the OC TME. Neutrophils re-enter the circulation with altered phenotypes following interaction. The middle panel demonstrates the use of Raman Spectroscopy and machine learning to diagnose OC. Un-interacted neutrophils (Control) and tumor associated neutrophils (TANs) were used as training data, to train the ANN algorithm. Blood from OC patients was used as testing data. Bottom panel shows Raman profiles of early and late-stage neutrophils. Using these phenotypes as training data, and blood of OC patients as testing data, OC could be clearly distinguished into early and late stages.

FIG. 21 shows the tumor associated immune cells in ovarian cancer TME. A shows a heatmap demonstrating immune landscape in ovarian cancer tumor microenvironment (n=430). In the ovarian tumor microenvironment, signaling between immune cells and ovarian cancer cells can affect immune responses and regulate cancer progression. B shows proportion of neutrophils in ovarian cancer tumor microenvironment. Neutrophils are the predominant inflammatory cells among the immune cells. C shows a schematic of a neutrophil cell on the nano sensor. From the peripheral blood, immune cells were isolated and 5 ul of each immune cells were dropped on the sensor and Raman spectra were taken. D shows Raman spectra of neutrophil, dendritic cell (D cell), monocyte, NK cell and T cell. E shows multiple clusters corresponding to different immune cells. PCA allows visualization of Raman signal variance between the cells. F shows the Hierarchical cluster analysis (HCT) confirming the distinct features of immune cells

FIG. 22 shows the discovery of a unique neutrophil cell signature specific to the presence of ovarian cancer. A shows a schematic of uninteracted (i) and interacted (ii) neutrophil on the nano sensor. B shows the Raman spectra of un-interacted (i) and tumor interacted (ii) neutrophils, showing lipid, protein, and nucleic acid contribution identified by grey boxes. C shows a schematic of neutrophil function through oxidative and non-oxidative mechanisms. D shows a Biplot of un-interacted and ovarian cancer interacted neutrophils. E shows a loading plot obtained from the PCA highlighting the significant peaks contributing to the classification. F shows hierarchical cluster analysis confirming the distinct clusters formed by uninteracted and tumor interacted neutrophils.

FIG. 23 shows variation in ovarian cancer cell interacted and ovarian cancer stem cell interacted neutrophils over time. A shows a schematic of heterogenous ovarian cancer tumor micro-environment with ovarian cancer and ovarian cancer stem cells and neutrophils. B and C show Raman spectra of ovarian cancer associated (B) and ovarian cancer stem cell associated (C) neutrophils in 0, 6, 12 and 24 hours, respectively. D and E show variations in neutrophil Raman signal upon interaction with ovarian cancer cells (D) and ovarian cancer stem cells (E) at different time points. From left to right are the Raman signals from carbohydrates, lipids, and proteins. F shows PCA highlighting the distinct clusters of ovarian cancer and cancer stem cell at 0 h (i), 6 h (ii). 12 h (iii), and 24 h (iv).

FIG. 24 shows the Raman signature of programmed death ligand 1 (PD-L1), and an evaluation of the PD-L1 peaks in early and late-stage neutrophils A shows the Raman signature of immune check point protein PD-L1, highlighting significant peaks. B shows a simulation of early and late-stage neutrophils using TGFβ (left, top) & all trans retinoic acid (left, bottom) and a comparison of Raman signatures of early-stage neutrophils and late-stage neutrophils with significant peaks of PDL-1 immune check point protein (right). C shows Raman signatures of early (left) and late-stage (right) neutrophils. D shows Student t-tests indicating significant (p<0.0001=****) differences between early and late-stage neutrophils at 546 cm⁻¹, 672 cm⁻¹, 853 cm⁻¹, 1004 cm⁻¹, 1452 cm⁻¹, 1589 cm⁻¹, 1672 cm⁻¹ (from left to right).

FIG. 25 shows the ability of the model to predict healthy versus ovarian cancer status using patient blood. A) Schematic of OC detection using machine learning based diagnostic model with scatter plot prediction. B) Sensitivity and specificity curve for OC detection. C) Calibration curve vs predicted probability. D) Classification accuracy curve for OC detection. E) Neural network scatter plot indicating clear clusters between healthy and cancer. F) Sensitivity and specificity curve using blood-based training data sets. G) Neural network scatter plot for OC detection, wherein using OC patient data as both testing and training (80:20) yields 100% sensitivity and 100% specificity with Neural Network.

FIG. 26 shows the ability of unique neutrophil signatures to classify early versus late-stage cancer. A) Schematic of early OC detection using machine learning based diagnostic model. B) Sensitivity and Specificity curve for early ovarian cancer detection. C) Calibration curve vs predicted probability. D) Classification accuracy curve for early diagnostic model. The model has an area under the curve of 1.000, classification accuracy of 0.900, sensitivity of 0.900, specificity of 1.000, precision of 0.917, and recall of 0.900. E) Scatter plot of early and late-stage ovarian cancer using indicating clear clusters.

FIG. 27 shows in vitro validation of T cell DNA as biomarkers for early detection of breast cancer. (a) Schematic of experimental design (left) and SERS profiles (right) of DNA isolated from healthy T cells and T cells interacted with breast cancer stem cells showing a clear distinction of peak values. (b) PCA scatter plot showing the distinct differences between the SERS signatures of DNA from healthy T cells and T cells associated with breast cancer stem cells (BCSCs). (c) PC loading plots for principal components PC1, PC2, PC3 and PC4 of the PCA analysis showing the contributing peaks for the differences observed between healthy and BCSC associated T cells.

FIG. 28 shows a similarity analysis between breast cancer stem cell associated T cell DNA, breast cancer tumor associated T cell DNA, and the cell free DNA present in the liquid biopsy sample. (a) Schematic of experimental design. (b) tSNE analysis demonstrating similarity between liquid biopsy and breast cancer CSC associated T cell DNA. (c) tSNE analysis demonstrating similarity between serum and tumor DNA. (d) tSNE analysis demonstrating similarity between breast cancer stem cell associated T cell DNA and tumor DNA. (e) tSNE analysis, (f) dendrogram (left) and heatmap (right), and (g) hierarchical clustering, all showcasing the similarity between breast cancer stem cell associated T cell DNA, cell free DNA in serum, and tumor DNA, confirming the ability to apply immune cell associated cell free DNA for cancer diagnosis.

FIG. 29 shows the validation of nano sensors for early diagnosis of breast cancer using different sized organoid cultures. (a) Illustration showing different sized organoid cultures mimicking the different grades of tumor, cultured by seeding (i) 10 million cells (ii) 1 million cells (iii) 10,000 cells (iv) 1000 cells. (b) SERS signatures of DNA isolated from T cells interacted with differed sized breast cancer tumor organoids. (C) Light microscopy images showing the formation of breast cancer tumor organoids in aggrewell plate. (d) tSNE analysis confirming discrete clusters. (e)-(i) Comparisons of intensities of SERS spectra of the T cell DNA interacted with the breast cancer tumor organoids at the peak positions: (e) 1003 cm⁻¹, (f) 1142 cm⁻¹, (g) 1208 cm⁻¹, (h) 1342 cm⁻¹, and (i) 1492 cm⁻¹.

FIG. 30 shows the applicability cell free DNA from T cells for diagnosis in cancer. (a) and (b) Schematic representations of training data experimental design (a) and testing data experimental design (b). (c) Scatter plot showcasing the probability of a sample to be diagnosed as early-stage cancer (compared to healthy) based on T cell-associated cell-free DNA signature. (d) Calibration curve showing the performance of machine learning model for diagnosis of early-stage cancer using T cell-associated cell-free DNA signature, with a sensitivity of 93.6% and a specificity of 100%. (e) Principal components contributing to accurate early-stage cancer diagnosis. (f) ROC curve for early-stage cancer prediction. (g) Schematic of cancer staging experiments using T cell-associated cell-free DNA signature. (h) Scatter plot showcasing the probability of a sample to be diagnosed as late-stage cancer (as compared to early stage) based on T cell-associated cell-free DNA signature. (i) Calibration curve showing the performance of the machine learning model for diagnosis of late-stage cancer using T cell-associated cell-free DNA signature, with a specificity of 86.8% and a sensitivity of 99%. (j) ROC curve for late-stage cancer prediction. (k) Schematic of cancer cell differentiation diagnosis using T cell-associated cell-free DNA signature. (l) Scatter plot showcasing the probability of a sample to be from low differentiation, medium differentiation, or high differentiation based on T cell-associated cell-free DNA signature. (m) Calibration curve showing the performance of the machine learning model for diagnosis of state of cancer differentiation using T cell-associated cell-free DNA signature, with a specificity of 91.3.8% and a sensitivity of 98.2%. (n) ROC curve for state of cancer differentiation prediction.

FIG. 31 shows the unique immunogenomic signature of breast cancer. a) Schematic representation of the experimental setup to determine the immunogenomic signature of breast cancer. b) SERS spectral signatures showcasing the characteristic peaks associated with DNA. c) Scatter plot showcasing the differentiation of immunogenomic signature associated with breast cancer compared to no cancer using clustering analysis. d) Loading plot for principal components, namely PC1 and PC2, showing maximum variance. e) Violin plot showcasing the distribution of F1 score calculated using discriminant analysis. f) Heatmap (bottom) and dendrogram (top) showcasing the correlation and heterogeneity in the immunogenomic signature of breast cancer and non-cancer.

FIG. 32 shows that the immunogenomic signature differs between subtypes of breast cancer associated with HER2 expression. a) Schematic representation of the experimental setup to determine the immunogenomic signature of breast cancer subtypes based on HER2 expression. b) Representative SERS spectra showing the immunogenomic signature of HER2 positive breast cancer and HER2 negative breast cancer. c) Box plots comparing the normalized intensities of characteristic peaks associated with DNA in HER2 positive breast cancer and HER2 negative breast cancer. d) Correlation matrix showcasing the linear correlation between the normalized peak intensities associated with HER2 positive breast cancer and HER2 negative breast cancer. e) Multidimensional scaling analysis to assess the similarities and differences between the immunogenomic signature of HER2 positive breast cancer and HER2 negative breast cancer.

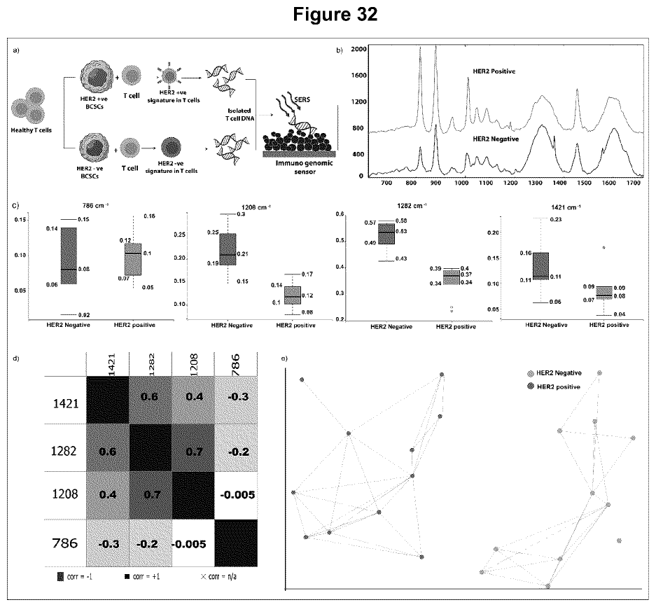

FIG. 33 shows that HER2 positive metastatic breast cancer exhibits a distinctive immunogenomic signature as compared to HER2 positive primary breast cancer. a) Schematic representation of the experimental model used to establish the immunogenomic signature of HER2 positive primary and metastatic breast cancer. b) Representative SERS spectra of HER2 positive primary breast cancer and HER2 positive metastatic breast cancer. c) Graph network analysis showcasing the difference between HER2 positive primary breast cancer and HER2 positive metastatic breast cancer based on immunogenomic signature. d) Box plots comparing the normalized SERS intensity of the DNA peaks contributing to the differences in the immunogenomic signature of HER2 positive primary breast cancer and HER2 positive metastatic breast cancer. For each comparison in d) the groups were statistically different, with p values <0.001 to <0.0001 using Student's t tests.

FIG. 34 shows a validation of the exclusiveness of the immunogenomic signature of metastatic breast cancer in clinical samples. a) Hierarchical clustering (left) and violin plot (right) showing the similarity of DNA derived from a tissue biopsy of metastatic breast cancer and the HER2 positive metastatic breast cancer profile. b) Graph network analysis of serum from clinical samples of patients diagnosed with metastatic breast cancer compared to the HER2 positive metastatic breast cancer profile. c) Hierarchical clustering (left) and violin plot (right) showing the differences in DNA derived from a tissue biopsy of metastatic breast cancer and the HER2 positive primary breast cancer profile, d) Graph network analysis of serum from clinical samples of patients diagnosed with metastatic breast cancer compared to the HER2 positive primary breast cancer profile.

FIG. 35 shows the deconvolution of SERS spectral data using synthetic mixtures. a) SERS spectra of tumor DNA mixed with varying percentages of immune cell DNA. b) SERS spectra of epithelial DNA mixed with varying percentages of immune cell DNA. c) Linear dependence of the characteristic peak intensities with change in immune cell DNA percentage in the mixture. d) Linear regression analysis to determine the cell types using immunogenomic signature. e) Estimate and actual cell-type proportions for each percentage of immune cell DNA. f) Synthetic mixture data was used to train the algorithm, which was validated using an independent cohort of clinical tissue biopsy samples (left) and liquid biopsy samples (right).

FIG. 36 shows that a metastatic immunogenomic signature can accurately predict metastasis of breast cancer and whether metastasis is intracranial. a) Schematic of the experimental design. b) Model prediction of HER2 positive phenotype in an independent cohort of clinical samples. c) Performance of the machine learning model assessed using specificity (99.3%) and sensitivity (97.3%) of prediction. d) Decision tree showcasing the predominant features of the data contributing to accurate prediction of HER2 positive phenotype. The HER2 positive immunogenomic signature can accurately classify the phenotype of breast cancer. e) Model prediction of metastatic phenotype in an independent cohort of clinical samples. f) Performance of the machine learning model assessed using specificity (97.2%) and sensitivity (100%) of classification algorithm. g) Decision tree showcasing the predominant features of the data contributing to precise classification of metastatic phenotype. h) Model prediction of intracranial site of metastasis in an independent cohort of clinical samples. i) Performance of the machine learning model. J) Principal components contributing to accurate prediction of intracranial site of metastasis.

FIG. 37 shows that NK cell DNA signature can distinguish between tumor interacted and non-tumor interacted phenotypes A shows a schematic of the experimental design using the sensor to generate Raman spectra. B shows Raman spectra of un-interacted (bottom), and tumor interacted (top) NK cell DNA. C shows univariate analysis using t-tests comparing between major peaks in the two phenotypes. * indicates p<0.05. ** indicates p<0.01. *** indicates p<0.001. D shows a principal component analysis (PCA) showing clear clustering of the two samples. E shows a hierarchical clustering analysis further supporting the clear separation of clusters seen in PCA, indicating that the phenotypes are distinct.

FIG. 38 shows that NK cell DNA methylation signature can distinguish between colorectal tumor interacted vs non-interacted phenotypes A shows the optimal standard curve generated with 5-mC standard control based on OD at 450 nm. B shows a linear correlation between peak intensity and methylation percentages. C show the SERS spectra of 5-mC with different methylation percentages. As the methylation percentage increases, the peak intensity is seen to rise. D shows colorimetric and Raman calculation for comparison of methylation specific peaks (765, 924, 1020, 1310 and 1555) for colo205-interacted & healthy control (HCT). E shows a comparison of methylation peaks in colorectal cancer interacted NK cell DNA and healthy NK cell DNA. F shows a Principal Component Analysis with methylation specific peaks. G shows a comparison of Raman spectra of NK cell methylation specific peaks in colorectal cancer patients and healthy patients. Increased immune cell methylation is seen in colorectal cancer patients.

FIG. 39 shows that NK cell methylation signature can distinguish between colorectal cancer patients and healthy volunteers A shows Raman spectra of colorectal cancer patients' blood (buffy) (n=7). B shows Raman spectra of healthy volunteers' blood (buffy) (n=10). C shows a scree plot for determining the number of principal components to be chosen. Using PC1 and PC2 gives the maximum variance. D shows a principal component analysis showing clear separation of clusters using Raman spectra of colorectal cancer patients' blood (buffy) and healthy volunteers' blood (buffy). E shows a loading plot showing characteristic peaks differentiating healthy and cancer patients Raman spectra. F shows a heat map showing differences in spectral intensity between Raman spectra of colorectal cancer patients' sample and healthy volunteers' sample.

FIG. 40 shows an exploratory analysis with augmented data of cell cultures with DNA structure and methylation peaks, wherein the cell cultures produced in vitro colorectal tumor interacted NK cells and tumor un-interacted NK cells. (A) PCA showing clear separation of clusters of un-interacted, and tumor interacted DNA. (B) Biplot for diagnosis. Features of healthy controls used for classification were 1276 cm⁻¹, 1358 cm⁻¹, 1515 cm⁻¹, 1662 cm⁻¹ and 1673 cm⁻¹. The features of colorectal cancer used for classification were 765 cm⁻¹, 924 cm⁻¹, 1011 cm⁻¹, 1123 cm⁻¹, 1310 cm⁻¹, 1555 cm⁻¹, 1560 cm⁻¹ and 1623 cm⁻¹. Then PLSDA was trained with the peaks for DNA structure and methylation in patient data. 10 spectra (5 from healthy and 5 from patient buffy coat) were kept separate as blind dataset for testing. C shows predicted ROC with AUC 1.0. PLSDA CV was venetian blinds and CV for ANN was 80:20 data. D shows ANN performance with model accuracy and model loss of ANN model.

FIG. 41 shows a schematic for detection of glioblastoma (GBM) through GBM associated NK cell derived extracellular vesicles and a supervised machine learning classifier. Left panel shows training data set collection with GBM-associated immune vesicle profile and tumor uninteracted immune vesicle profile as GBM and healthy classes, respectively. GBM associated immune vesicle profiles were obtained by coculture of NK92 with A172, U118, and T98 cells to represent cancer data set. Tumor uninteracted immune vesicle profiles were obtained from uninteracted NK92 to represent healthy data set. Middle panel shows model learning where both the training data sets were dimensionally reduced using PCA (principal component analysis), and then the PLSDA (partial least squares discriminant analysis) and ANN (artificial neural network) algorithms were applied. Right panel shows the blood-brain barrier (BBB) crossing circulating immune vesicle (CIV) biomarkers for cancer detection. Trained algorithms were validated with spectra from GBM cancer patients' serum and healthy donors' serum. With this model GBM was predicted with 96.82% sensitivity and 100% specificity with area under the curve (AUC) of 0.9568 with PLSDA and 100% sensitivity and specificity and AUC of 1.0 with ANN.

FIG. 42 shows a characterization of immune cells and their EVs, and identifies NK cell EV signatures as a candidate for diagnosing glioblastoma. (A) Natural killer cells infiltrate glioblastoma tumor. (i) Heat map showing immune landscape in glioblastoma tumor (n=174). (ii) Relative proportion of immune cell populations found in glioblastoma. (B) Relative population of immune cell infiltrating glioblastoma tumor with NK cells used in this study highlighted. (iii) Relative gene expression of immune cells seen in glioblastoma patients. (iv) Heat map showing immune cell gene expression among 174 glioblastoma samples. (B) Distinct spectral features of natural killer cell EVs in the mixed population of immune cells. (a) Schematic of immune cells and their extracellular vesicles interacting with the ImmunoProfiler Platform. (b) SERS spectra of individual immune cell extracellular vesicles. (i) B cell EV, (ii) dendritic cell EV, (iii) macrophage EV, (iv) NK cell EV, (v) T cell EV. (c) Natural killer cell EVs can be identified distinctly from the mix of immune cell EVs by PCA. (C) Similarity between NK cells and their EVs. (i) Schematic of extracellular vesicles (exosome and microvesicles) released by natural killer cells. (ii) Transmission electron microscopy (TEM) of NK cell and its associated extracellular vesicles. (iii) TEM of NKEVs with near spherical morphology, electron dense dark outer membrane, and electrolucent bright inner mass. (iv) and (v) SERS spectra of NK cell EV (iv) and NK cell (v). (vi) Chord diagram with red chords representing parent NK cell and blue chords representing NKEVs. The lower half of the circle represents the immune cell and its component. The interconnected chords show the similarity of NK cell and NKEVs. (D) Characterization of NK cell EVs. (i) Flow cytometry dot plot for CD56 positive NKEVs from healthy patient serum and histogram of the CD56 positive and negative population. (ii) Snapshot of NKEVs during measurement with mean concentration of 8.36×107 particles/mL and size of NKEVs ranging from 50 to 450 nm with a mean of 205 nm (n=101).

FIG. 43 shows the distinct phenotypes of CIVs associated with glioblastoma (GMB) and unassociated CIVs. (A) Schematic depicting the interaction of an immune cell (NK cell) with a tumor cell in the tumor microenvironment. Consumption of glucose and amino acids depletes the immune cell's fuel, and as a result, the immune cell undergoes metabolic reprogramming. Additionally, the fatty acids and lactate generated by the tumor cell acidify the tumor milieu, intensifying the reprogramming process. (B) SERS spectra of GBM associated immune cell vesicle and uninteracted immune cell vesicles. GBM associated immune cell vesicle spectra were obtained by coculturing A172, U118, and T98 GBM cells with NK-92 cells. (C) Bar graphs showing differences in pyruvate (left), lactate (middle), and glucose (right) between uninteracted and GBM associated vesicles and their corresponding regression plots (R2=0.9392 for glucose pyruvate and R2=0.9396 for glucose-lactate, P<0.0001). (D), (E), and (F) show PA for lipid, protein, and nucleic acid composition, respectively. Clear separation of clusters can be seen between the GBM interacted vesicles and uninteracted vesicles. (G), (H), and (I) show two tailed t test in lipids, proteins, and nucleic acids, respectively, between uninteracted immune cell vesicle and GBM associated immune cell vesicle. Increase in C—H vibration of lipids, skeletal stretching lipids, fatty acids, and CH2 vibration in lipids are observed to increase in GBM associated immune vesicles at 3015, 1064, 1142, and 2945 cm⁻¹, respectively. Increase in amide I (1672 cm⁻¹) and phenylalanine (1000 cm⁻¹) can be observed in uninteracted immune vesicles, while amide Ill (1235 cm⁻¹) and Ch2 deformation of proteins (1447 cm⁻¹) are increased in GBM associated immune vesicles (****=P<0.0001, ***=P<0.001, **=P<0.01).

FIG. 44 shows PDL-1 and CTLA-4 marker comparisons within GBM associated and unassociated NK cell EVs. (A) PDL1 and CTLA-4 signature Raman spectra. (B) Raman spectra of GBM associated immune vesicle (top) and uninteracted immune vesicle (bottom). (C) PDL1 and CTLA-4 peaks can be traced to 848 cm⁻¹, 855 cm⁻¹, 931 cm⁻¹, 936 cm⁻¹, 1121 cm⁻¹, 1250 cm⁻¹, 1335 cm⁻¹, 1344 cm⁻¹, 1463 cm⁻¹, 1460 cm⁻¹, 1658 cm⁻¹. (D) and (E) show PCA and biplot with respect to PDL1 and CTLA-4, respectively. (F) and (G) show relation of PDL1 (F) and CTLA-4 (G) expression in GBM associated immune vesicle and uninteracted immune vesicle with t tests, showing significant differences in PDL1 expression and CTLA4 between EV phenotypes. (H) Flow cytometry showing PDL1 (i) and CTLA-4 (ii) expression in GBM associated immune vesicle and uninteracted immune vesicle. ****=P<0.0001, ***=P<0.001, *=P<0.005.

FIG. 45 shows that unique signatures from NK cell EVs can be used to diagnose glioblastoma (GBM). (A) ImmunoProfiler identifies GBM signatures in NK cell derived extracellular vesicles and noncancer signatures from uninteracted NK cell derived extracellular vesicles. SERS spectra of EVs derived from NK-92 interacted with (top to second from the bottom) A172, U118, and T98 were used as input training data for cancer data set, and SERS spectra of uninteracted NK-92 cell derived EVs (bottom) were used as healthy control. Validation was performed with SERS spectra of serum samples from GBM patients (v) and healthy controls (vi). (B) Principal component analyses of uninteracted vesicles and GBM interacted vesicles show separate distinct clusters. (C) Partial least squares discriminant analysis showing score scatter plot for training model and validation model (n=20). (iii) Model prediction results with test data (n=20) from serum derived from GBM and healthy patients and assessment of model performance with ROC analysis. Sensitivity=96.82%, specificity=100%, AUC=0.9568. (viii) VIP scores with the variable of importance scores are plotted on x axis and corresponding variable is on y axis. (D) Artificial neural network (ANN) analysis with the results complementing PLSDA. ANN shows 100% sensitivity, specificity, accuracy, and precision.

FIG. 46 shows that circulating immune vesicles (CIVs) interacted with three brain cancers (astrocytoma, oligodendroglioma, and glioblastoma) show three distinct phenotypes. A SERS spectra of astrocytoma associated CIV. B SERS spectra of oligodendroglioma associated CIV. C Principal component analysis showing PC1 and PC2 score plots having separate clusters of oligodendroglioma, astrocytoma, and GBM associated CIVs. Red, glioblastoma associated CIVs. Top cluster is oligodendroglioma associated CIVs. Bottom right cluster is astrocytoma associated CIVs. Bottom left cluster is glioblastoma associated CIVs. D and € show loading plots of PC1 and PC2, respectively, showing classification markers for oligodendroglioma vs GBM and astrocytoma vs GBM, respectively. F Comparison of spectral markers among three different brain cancer types. G Hierarchical clustering analysis showing clear clusters for all three different types of brain tumors (astrocytoma, oligodendroglioma, and glioblastoma).

FIG. 47 shows the diagnosis of ovarian cancer with extracellular vesicles (EVs) of T cells associated with ovarian cancer. Top left shows Raman spectra of EVs from healthy T cells (bottom) and ovarian cancer associated T cells (top). Top right shows distinct Raman intensity changes featured with a heatmap (top). and a PCA plot (bottom) demonstrating clear clustering and thus the discrimination ability of this methodology. Left cluster is EVs from ovarian cancer associated T cells and right cluster is EVs from healthy T cells. Middle figure panel shows a PLSDA validation of the model with patient plasma demonstrating the ability to accurately classify ovarian cancer. Horizontal dashed bars represent prediction above 50%. Healthy plasma is indicated by light circles, whereas ovarian cancer plasma is indicated by dark circles. Bottom left shows an assessment of model performance in classifying a sample as healthy or ovarian cancer, with receiver operating characteristic curve (ROC) analysis. ROC demonstrated accuracy of classification, with area under the curve (AUC)=0.9412. Bottom right shows calibration curves showing that the predicted response sensitivity is 60% and specificity is 100%.

FIG. 48 shows that signatures of T cells associated with brain tumor can predict early vs late cancer diagnosis. Left shows Raman profiles of T cells associated with early brain cancer (bottom) and advanced brain cancer (top). Top right shows a principal component analysis demonstrating variation in the T cell signatures between early (right cluster) and late cancer (left cluster), enabling early brain cancer diagnosis. Bottom right shows hierarchical cluster analysis demonstrated distinct clusters in the dendrogram of T cells associated with early brain cancer (bottom cluster) vs T cells associated with advanced brain cancer (top cluster).

FIG. 49 shows that immune cell signatures can distinguish between benign and malignant cancer. Top left is a SERS spectrum of T cells associated with benign brain tumor. Top right is a SERS spectrum of T cells associated with malignant tumor. Middle shows that the distinct features of T cells associated with benign and malignant tumor form distinct clusters in PCA analysis. Bottom shows an ANN prediction model classifying benign and malignant tumor with 100% accuracy.

FIG. 50A is a block diagram showing steps of a method of cancer detection, according to at least one example embodiment described herein.

FIG. 50B is a block diagram of the hardware components used in obtaining Raman spectra of biomarkers on nanosensors during the method of FIG. 50A, according to at least one example embodiment described herein.

FIG. 50C is a block diagram of an example embodiment of a computing device and software that may be used with the hardware setup of FIG. 50B.

FIG. 50D is a flowchart of a method for performing a cancer assessment on a patient sample using Raman spectra obtained therefrom that may be performed by software of the computing device of FIG. 50C, according to at least one example embodiment described herein.

FIG. 50E is a flowchart of a method for performing training to obtain models that may be used by the software of the computing device of FIG. 50C in detecting various conditions from the patient sample.

Further aspects and features of the example embodiments described herein will appear from the following description taken together with the accompanying drawings.

### DESCRIPTION

Various apparatuses, methods and compositions are described below to provide an example of at least one embodiment of the claimed subject matter. No embodiment described below limits any claimed subject matter and any claimed subject matter may cover apparatuses and methods that differ from those described below. The claimed subject matter are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed subject matter. Any subject matter that is disclosed in an apparatus, method or composition described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the end result is not significantly changed. These terms of degree should be construed as including a deviation of the modified term, such as 1%, 2%, 5%, or 10%, for example, if this deviation does not negate the meaning of the term it modifies.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, for example, if the end result is not significantly changed.

It should also be noted that, as used herein, the wording “and/or” is intended to represent an inclusive-or. That is, “X and/or Y” is intended to mean X, Y or X and Y, for example. As a further example, “X, Y, and/or Z” is intended to mean X or Y or Z or any combination thereof. Also, the expression of A, B and C means various combinations including A; B; C; A and B; A and C; B and C; or A, B and C.

The term “subject” as used herein includes all members of the animal kingdom including mammals, and suitably refers to humans. Thus, the methods and uses of the present application are applicable to both human therapy and veterinary applications. In one embodiment, the subject is a patient.

The term “cell” as used herein refers to a single cell or a plurality of cells and includes a cell either in a cell culture or in a subject.

The example embodiments of the devices, systems or methods described in accordance with the teachings herein may be implemented as a combination of hardware and software. For example, the embodiments described herein may be implemented, at least in part, by using one or more computer programs, executing on one or more programmable devices comprising at least one processing element and at least one storage element (i.e. at least one volatile memory element and at least one non-volatile memory element). The hardware may comprise input devices including at least one of a touch screen, a keyboard, a mouse, buttons, keys, sliders and the like, as well as one or more of a display, a speaker, a printer, and the like depending on the implementation of the hardware.

It should also be noted that there may be some elements that are used to implement at least part of the embodiments described herein that may be implemented via software that is written in a high-level procedural language such as object oriented programming. The program code may be written in MATLAB, Julia, Python, C, C⁺⁺ or any other suitable programming language and may comprise modules or classes, as is known to those skilled in object oriented programming. Alternatively, or in addition thereto, some of these elements implemented via software may be written in assembly language, machine language or firmware as needed. In either case, the language may be a compiled or interpreted language.

At least some of these software programs may be stored on a computer readable medium such as, but not limited to, a ROM, a magnetic disk, an optical disc, a USB key and the like that is readable by a device having a processor, an operating system and the associated hardware and software that is necessary to implement the functionality of at least one of the embodiments described herein. The software program code, when read by the device, configures the device to operate in a new, specific and predefined manner in order to perform at least one of the methods described herein.

Furthermore, at least some of the programs associated with the devices, systems and methods of the embodiments described herein may be capable of being distributed in a computer program product comprising a computer readable medium that bears computer usable instructions, such as program code, for one or more processing units. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

The following description is not intended to limit or define any claimed or as yet unclaimed subject matter. Subject matter that may be claimed may reside in any combination or sub-combination of the elements or process steps disclosed in any part of this document including its claims and figures. Accordingly, it will be appreciated by a person skilled in the art that an apparatus, system or method disclosed in accordance with the teachings herein may embody any one or more of the features contained herein and that the features may be used in any particular combination or sub-combination that is physically feasible and realizable for its intended purpose.

Recently, there has been a growing interest in developing new technologies of early cancer detection and prognosis. Specifically, there has been a growing interest in identifying new biomarkers in physiological (for example, blood) samples from humans for early cancer detection and prognosis. In particular, the present disclosure relates to the use of tumor associated immune cells as biomarkers for cancer.

Accordingly, in one embodiment provided herein is a method of providing a cancer assessment for a subject, the method comprising:
measuring a value of one or more cancer traits of at least one tumor associated immune cell (TAIC) or TAIC-derived biomarker in a fluid sample obtained from the subject to obtain a test profile;
comparing the test profile to one or more reference profiles;
wherein similarity or a differential in the test profile to the one or more reference profiles is indicative of one or more cancer characteristics; and
based on the one or more cancer characteristics, providing the cancer assessment of the subject.

Also provided herein is a method of determining one or more cancer characteristics for a subject, the method comprising:
measuring a value of one or more cancer traits of at least one tumor associated immune cell (TAIC) or TAIC-derived biomarker in a fluid sample obtained from the subject to obtain a test profile;
comparing the test profile to one or more reference profiles;
wherein similarity or a differential in the test profile to the one or more reference profiles is indicative of one or more cancer characteristics.

As used herein, the term “tumor associated immune cell” or “TAIC” refers to an immune cell that is, or has been, present in the microenvironment of a tumor. The “microenvironment of a tumor” or “tumor microenvironment” (TME) refers to the environment around a tumor, including the surrounding blood vessels, immune cells, fibroblasts, signaling molecules and the extracellular matrix (ECM).

Immune cells include but are not limited to T cells, Natural Killer (NK) cells, macrophages, neutrophils, myeloid derived suppressor cells (MDSCs), dendritic cells (DCs), B cells and mast cells.

TAICs include, but are not limited to tumor associated T cells, including cancer cell associated T cells, cancer stem cell associated T cells, and tumor associated stem cell like T cells; tumor associated NK cells, including cancer cell associated NK cells, cancer stem cell associated NK cells, and tumor associated stem cell like NK cells; tumor associated macrophages, including cancer cell associated macrophages, cancer stem cell associated macrophages, and tumor associated stem cell like macrophages; tumor associated neutrophils, including cancer cell associated neutrophils, cancer stem cell associated neutrophils, and tumor associated stem cell like neutrophils; tumor associated dendritic cells, including cancer cell associated dendritic cells, cancer stem cell associated dendritic cells, and tumor associated stem cell like dendritic cells; and tumor associated B cells, including cancer cell associated B cells, cancer stem cell associated B cells, and tumor associated stem cell like dendritic cells.

As used herein, the term “tumor associated immune cell (TAIC)-derived biomarker” refers to a biomarker derived from or associated with a tumor associated immune cell. Examples of “tumor associated immune cell (TAIC)-derived biomarkers” include, but are not limited to, extracellular vesicles of tumor associated immune cells, cell-free DNA from tumor associated immune cells and methylated DNA from tumor associated immune cells. Other examples of “tumor associated immune cell (TAIC)-derived biomarkers” include metabolic changes, activation status and surface phenotypes of TAICs.

In one embodiment, the TAIC or TAIC-derived biomarker is circulating in the patient. Accordingly, in one embodiment, the method comprises obtaining a fluid sample from the subject. The fluid sample is optionally blood, plasma, serum, urine, stool, mucus, saliva, cerebrospinal fluid and/or amniotic fluid. In one embodiment, the fluid sample is blood.

In another embodiment, the TAIC or TAIC-derived biomarker is present in the tissue of the patient. Accordingly, in one embodiment, the method comprises obtaining a tissue sample from the subject, optionally by tissue biopsy.

As used herein, the expression “cancer trait” refers to a property of, or associated with, a TAIC or TAIC-derived biomarker that is indicative of, or correlated with, the presence or absence of cancer. In one embodiment, the “cancer trait” is a measurable value.

For example, in one embodiment, the cancer trait is a Raman spectrum of at least one TAIC or TAIC-derived biomarker. In one embodiment a Raman spectrum is obtained using the methods described in detail herein as well as in PCT Application No PCT/CA2022/050232, the contents of which are incorporated herein in their entirety.

According to the methods described herein, a value of one or more cancer traits is measured to obtain a test profile (also referred to herein as a sample profile). The test profile may be a single measured value or multiple measured values. In one embodiment, the test profile is a Ramen spectrum, optionally a Surface-enhanced Raman spectroscopy (SERS) spectrum.

In one embodiment, a value of one or more cancer traits is measured by DNA Next gen sequencing; PCR; repertoire sequencing; Immunosequencing; single cell RNA sequencing; DNA methylation sequencing; unique molecular identifier (UMI); optical microscopy techniques, optionally confocal microscopy, immunohistochemistry, or super resolution microscopy; flow cytometry; or mass cytometry.

The method further comprises determining the level of similarity of said test profile to one or more reference profiles (also referred to herein as templates or template profiles).

The reference profile may be a reference value and/or may be derived from one or more samples, optionally from historical data for a subject or pool of subjects who are known to have, or not have, cancer or having specific type, grade or stage of cancer. The historical data can be a value that is continually updated as further samples are collected and subjects are identified as having or not having cancer, or having specific type, grade or stage of cancer. For example, the control database may be stored on an online database, which is continually updated with data from diagnosed patients.

The reference profiles are optionally derived from one or more reference TAICs. In one embodiment, reference TAICs are immune cells, for example immune cells from a healthy subject (also referred to herein as “naïve immune cells”), which are cultured in the presence of with a tumor and/or cancer cell line until the one or more reference tumor associated immune cell (TAICs) expresses one or more cancer traits.

In another embodiment, reference TAICs are immune cells obtained from a subject with cancer.

A reference profile generated from one or more reference TAICs is an example of a “cancer specific reference profile”. A reference profile generated from one or more naïve immune cells is an example of a control reference profile, a healthy reference profile, or a “non-cancer specific reference profile”.

Where the reference profile is a cancer specific reference profile (for example, a reference profile that is indicative of cancer or a cancer with one or cancer characteristics), (i) a high level of similarity of the test profile to the cancer specific reference profile; and/or (ii) a higher level of similarity of the test profile to the cancer specific reference profile than to a non-cancer specific reference profile indicates the presence of, or an increased likelihood of, cancer or cancer with the one or more cancer characteristics.

Where the reference profile is a non-cancer specific reference profile (for example, a reference profile that is not indicative of cancer), (i) a low level of similarity of the test profile to the non-cancer specific reference profile; and/or (ii) a lower level of similarity to the non-cancer specific reference than to a cancer specific reference profile indicates the presence of, or an increased likelihood of, cancer or cancer with one or more cancer characteristics.

A “cancer characteristic” as used herein can include, but is not limited to, the presence or absence of cancer, a cancer type, a cancer stage, a cancer grade, a primary cancer, a metastatic cancer, cancer potential for metastasis, a molecular phenotype of the cancer, a benign cancer, a malignant cancer, the tissue of origin of the cancer, the metastatic location of the cancer or a combination thereof.

In one embodiment, a higher level of similarity to the cancer specific reference profile than to the non-cancer specific reference profile is indicated by a closer clustering between the test profile and the cancer specific reference profile than clustering between the test profile and the non-cancer specific reference profile, as measured, for example by unsupervised multivariate analysis (principal component analysis or PCA).

Likewise, a lower level of similarity to a non-cancer specific reference than to a cancer specific reference profile is indicated by a less close clustering between the test profile and the non-cancer specific reference profile than clustering between the profile and the cancer specific reference profile, as measured, for example by unsupervised multivariate analysis (principal component analysis or PCA). “Close clustering” can be indicated for example by two profiles falling within one cluster or forming their own separate cluster.

In another embodiment, a higher level of similarity to the cancer specific reference profile than to the non-cancer specific reference profile is indicated by a higher correlation value computed between the test profile and the cancer specific reference profile than an equivalent correlation value computed between the test profile and the non-cancer specific reference profile, optionally wherein the correlation value is a correlation coefficient.

In another embodiment, a lower level of similarity to a non-cancer specific reference than to a cancer specific reference profile is indicated by a lower correlation value computed between the test profile and the non-cancer specific reference profile than an equivalent correlation value computed between the test profile and the cancer specific reference profile, optionally wherein the correlation value is a correlation coefficient.

Without being bound by theory, a reference profile for the cancer characteristic “metastatic cancer” according to the methods described herein can be used to identify an early stage sample or a preliminary diagnostic sample with the cancer characteristic “cancer potential for metastasis”.

In another embodiment, the correlation coefficient is a linear correlation coefficient, optionally a Pearson correlation coefficient or a Spearman correlation coefficient.

The term “cancer” as used herein refers to cellular-proliferative disease states.

In one embodiment, the cancer is brain cancer, optionally glioblastoma, astrocytoma, or oligodendroglioma. In another embodiment, the cancer is ovarian cancer. In another embodiment, the cancer is bladder cancer, breast cancer, colon cancer, esophageal cancer, gastric cancer, hepatic cancer, intestinal cancer, lung cancer, ovarian cancer, head and neck cancer, rectal cancer, prostate cancer, pancreatic cancer, thyroid cancer, cervical cancer, melanoma, nasopharyngeal cancer, testicular cancer, or uterine cancer.

In one embodiment, the breast cancer is HER2+ breast cancer, triple positive breast cancer or triple negative breast cancer.

In one embodiment, the cancer is an adult cancer or a pediatric cancer.

As used herein, the term “cancer assessment” includes, but is not limited to, any assessment that that may be provided to a patient or a care provider, including for example providing a presence of cancer, a cancer type, a cancer location, a stage of the cancer, a grade of the cancer, a metastatic potential of the cancer, or a cancer therapy efficacy. “Cancer assessment” also includes providing a prognosis for the patient, early cancer diagnosis, determining whether a tumor is benign or malignant, determining a grade of the cancer, determining whether a tumor is primary or metastatic, determining whether a primary tumor has potential for metastasis, determining a progression of the cancer, determining a nodal metastasis of the cancer, determining a clinical metastasis of the cancer, predicting patient survival, providing a prognosis for the patient, providing an early diagnosis of cancer, including providing an early diagnosis of hard to detect cancers, determining a presence of an aggressive brain cancer, providing a location of a tumor, monitoring cancer recurrence during or after therapy, and/or determining a presence of minimal residual disease.

The present disclosure also provides a method of treating a subject diagnosed with cancer, comprising: (a) providing a cancer assessment of the subject according to the methods as described herein and (b) treating the subject.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. A subject with early cancer can be treated to prevent progression for example, or alternatively a subject in remission can be treated to prevent recurrence. Treatment methods can comprise administering to a subject a therapy such as a chemotherapeutic drug, radiation and/or preforming surgery on the subject.

As used herein, “treating a cancer” includes, but is not limited to, reversing, alleviating or inhibiting the progression of the cancer or symptoms or conditions associated with the cancer. “Treating the cancer” also includes extending survival in a subject. Survival is optionally extended by at least 1, 2, 3, 6 or 12 months, or at least 2, 3, 4, 5 or 10 years over the survival that would be expected without treatment with a cytotoxic agent or composition as described herein. “Treating the cancer” also includes reducing tumour mass and/or reducing tumour. Optionally, tumour mass and/or tumour burden is reduced by at least 5, 10, 25, 50, 75 or 100% following treatment with a cytotoxic agent or composition as described herein. “Treating the cancer” also includes reducing the aggressiveness, grade and/or invasiveness of a tumour.

The application also provides a method of generating a reference tumor associated immune cell (TAIC) for use in cancer assessment, the method comprising coculturing one or more naïve immune cells with a tumor and/or cancer cell line until the one or more reference tumor associated immune cell (TAICs) expresses one or more cancer traits.

In one embodiment, the one or more naïve immune cell comprises a T cell, an NK cell, a macrophage, a neutrophil, a dendritic cell, or a B cell, or a combination thereof.

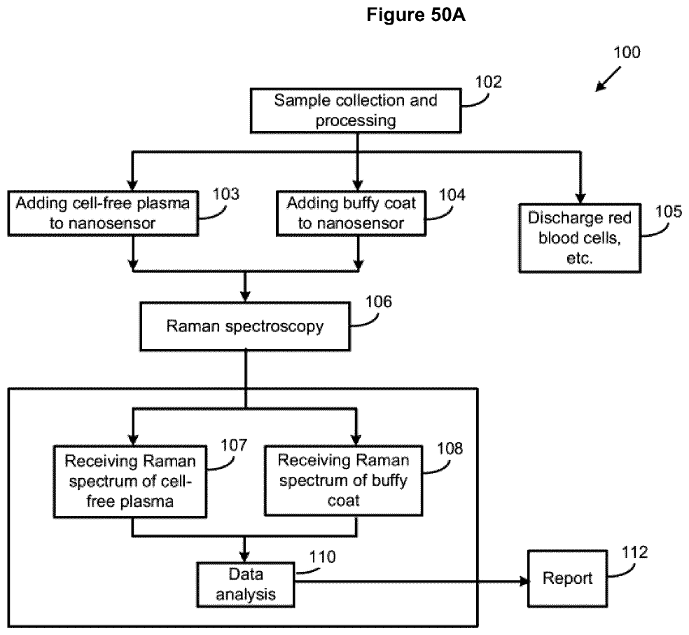

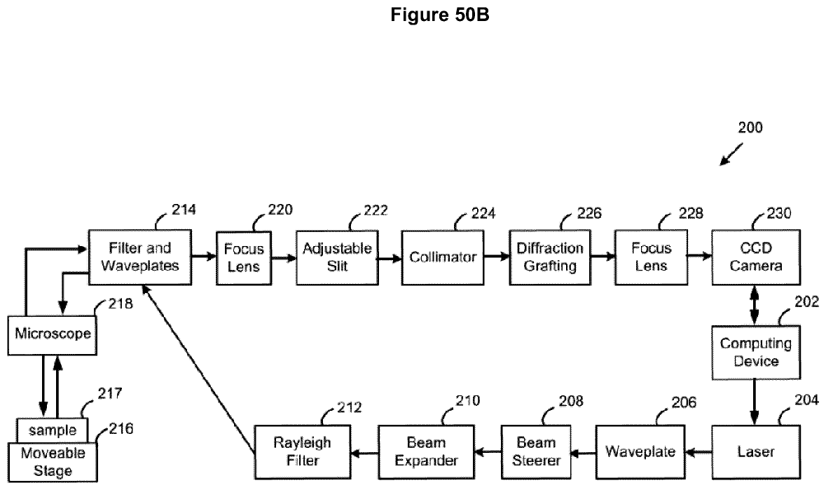

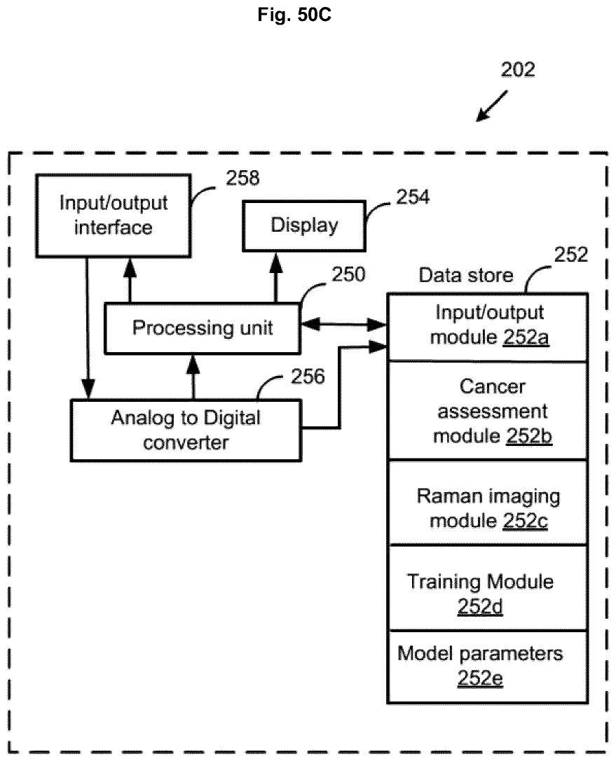

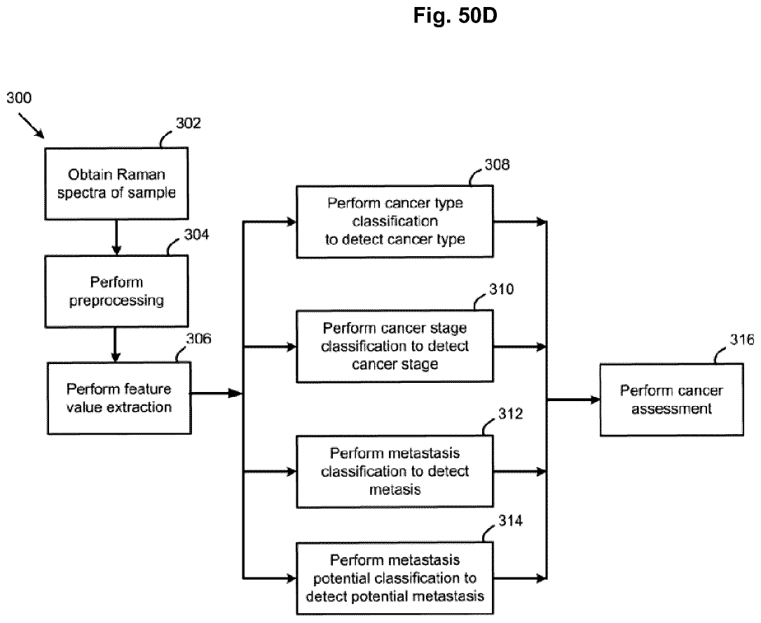

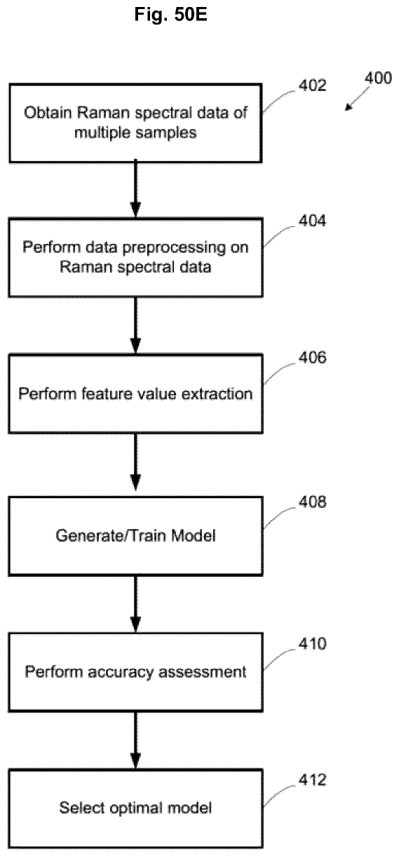

Referring now to the figures, FIG. 50A shows an example embodiment of a method 100 of assessing cancer in a subject in accordance with the teachings herein. FIG. 50B shows an example embodiment of a system 200 for obtaining Raman spectra of biomarkers captured by nanosensors, as described in the methods described herein.

In FIG. 50A, at step 102, a fluid sample is collected (e.g. raw blood plasma; plasma that has been collected from a subject) and processed to produce a volume of cell-free plasma, a volume of buffy coat and a volume of remaining products (e.g. red blood cells, etc.). The processing of step 102 may be done using suitable techniques such as, but not limited to, density gradient centrifugation, for example, to obtain the cell-free blood plasma, buffy coat and remaining products (e.g. red blood cells, etc.)

In at least one embodiment, at step 103, a volume of the blood plasma (e.g. cell-free blood plasma) produced from step 102 is dropped onto a first nanosensor for detection of cell-component biomarkers. In at least one embodiment, the volume of the blood plasma comprises at least one tumor associated immune cell (TAIC) or one tumor associated immune cell (TAIC)-derived biomarker.

In at least one embodiment, the volume of blood plasma added to the nanosensor is in a range of about 10 microliters (μL).

The nanosensors used herein for surface enhanced Raman scattering for biomolecule detection include one or more nanoparticles (e.g. nanoprobes). The nanoparticles are made of materials such as but not limited to gold, silver, platinum, titanium, silicon, aluminum, nickel, and/or graphite. The nanoparticles described herein may also be referred to as quantum dots (i.e. nanoparticles with particle size less than about 5 nm).

Unlike all other types quantum dots, the quantum dots of nanosensors described herein are generally non-toxic, making them particularly suitable for biomedical applications. In addition, the dots are free of contaminations and generally do not react/interfere with target molecules.

The quantum dots of the nanosensors described herein are smaller and have a unique structure (e.g. have a high vacancy density of crystalline nanoparticles) compared to conventional quantum dots, which translates to higher detection sensitivity and pushes the limit of detection to a lower concentration. Therefore, previously undetectable traces (undetectable because of their low concentration) may be detected using quantum dots of the nanosensors described herein.

In at least one embodiment, the nanosensor may amplify extremely weak signals of the one or more TAICs or TAIC-derived biomarkers. The nanosensor includes one or more self-assembled, three dimensional nanoprobes that can detect a biomolecular configuration of extremely low levels The nanosensor is a three-dimensional and porous network of nanoprobes. The interconnected crisscross of nanosensors can act as a trapping and screening device. In addition to biocomponents getting trapped by the nanosensor, fluids are easily drained from the nanosensor, thereby improving the interaction of the biocomponents with the nanosensors.

Generally, the nanosensors used in the methods and systems described herein have a three-dimensional (3D) structure comprising self-assembled closed rings and bridges which causes nanoparticles to aggregate together. Between the self-assembled closed rings and bridges are pores that are interconnected. The ring size is roughly the wavelength of a laser beam used to create the sensor.

In at least one embodiment, the nanoparticles contain rich crystalline defects. In at least one embodiment, the nanoparticle size is tuneable from about 100 nm to about 1 nm. For example, in at least one embodiment, the nanoparticle size may be less than about 1 nm.

In at least one embodiment, the nanosensors used in the methods and systems described herein are fabricated by the methods described in U.S. Provisional Patent Application No. 63/059,079 filed 30 Jul. 2020 entitled “ULTRASHORT LASER SYNTHESIS OF NANOPARTICLES OF ISOTOPES”, the contents of which are incorporated herein by reference.

In one embodiment, at step 104, optionally, a volume of the buffy coat produced from step 102 may be dropped onto a second nanosensor to detect immune-cell biomarkers. In at least one embodiment, the volume of buffy coat comprises one or more TAICs or TAIC-derived biomarkers.

In at least one embodiment, the volume of the buffy coat added to the nanosensor is in a range of about 10 uL.

In accordance with the teachings herein, the nanosensors that are used allow for the detection of TAICs or TAIC-derived biomarkers in low concentrations.

Optionally, at a step 105, the remaining products produced during step 102 may be discarded.

At step 106, in at least one embodiment, after a first incubation time, the first nanosensor including the volume of blood plasma is scanned under a Raman microscope to obtain a Raman spectrum.

In at least one embodiment, the first incubation time is in a range of about 1 to about 2 minutes. The length of the first incubation time is based on the time needed for the biomarkers to adsorb onto the surface of the nanosensors. The first incubation time may be determined empirically.

Also at step 106, in at least one embodiment, after a second incubation time, the second nanosensor including the volume of buffy coat is scanned under a Raman microscope to obtain a Raman spectrum.

In at least one embodiment, the second incubation time is in a range of about 1 to about 2 minutes. Again, the length of the second incubation time is based on the time needed for the biomarkers to adsorb onto the surface of the nanosensors. The second incubation time may be determined empirically.

At step 107 one or more Raman spectra of the volume of cell-free plasma on the nanosensor is generated by a Raman spectroscopy system.

At step 108 one or more Raman spectrum of the volume of buffy coat on the nanosensor is generated by a Raman spectroscopy system.

One example of a Raman spectroscopy system for obtaining Raman spectra of the samples on a nanosensor of steps 107 and/or 108 of method 100 is provided in FIG. 50B. FIG. 50B shows on example embodiment of a system 200 having one or more hardware components used in obtaining Raman spectra of biomarkers on nanosensors during, for example, step 106 of the method of FIG. 50A, according to at least one example embodiment described herein.

The system 200 includes a computing device 202, an excitation pathway including a laser 204, a waveplate 206, a beam steerer 208, a beam expander 210, a Rayleigh filter 212, filter and waveplates 214, and a moveable stage 216. The nanosensor is on the moveable stage 216. The system 200 further includes a return pathway including a microscope 218, the filter and waveplates 214, a focus lens 220, an adjustable slit 222, a collimator 224, a diffraction grating 226, a focus lens 228 and a CCD camera 230. In at least one embodiment, the system 200 is a confocal Raman microscope system with, for example, an excitation wavelength of 785 nm. This microscope system provides all of the components shown in FIG. 50B except for the computing device 202.

During use, the computing device 202 includes one or more software programs (see e.g. FIG. 50C) for performing Rayleigh imaging and analysis of a sample 217, such as the plasma or buffy coat sample, that has been placed on the nanosensor. Specifically, the computing device 202 is coupled to the laser 204 and the CCD camera 230 through I/O hardware including possibly an Analog to Digital Converter and/or USB cables, so that the computing device 202 may control the generation of laser pulses by the laser 204 and the recordal of images by the CCD camera 230. Laser pulses from the laser 204 are transitory optical beams of light that are polarized by the waveplate 206 and then steered by the beam steerer 208. The steered optical beams are then enlarged in diameter by the beam expander 210 and then filtered by the Rayleigh filter 212 and the filter and waveplates 214 to purify scattered light by which is then provided to the microscope 218, which may be a standard microscope, that includes focusing lens for focusing the excitation light pulses onto the sample 217.

The excitation light pulses excite molecules in the sample 217 and the excited molecules which emit scattered photons that are at different energies and different frequencies. There is also a change in the electric dipole-electric dipole polarization and a resulting Raman scattering which is proportional to the polarization change. The scattered photons, referred to as a Raman signal, are then focused and purified by the optical elements in the return pathway prior before reaching the CCD camera 230. The CCD camera 206 then records the images and transmits the images to the computing device 202 so that it can perform processing, as described below.

For example, referring now to FIG. 50C, shown therein is an example embodiment of a block diagram of an example embodiment of a computing device 202 and software that is used with the hardware setup of FIG. 50B. It should be noted that this is one example embodiment and in other embodiments the computing device 202 may have more or less components or alternative layouts in other embodiments. The computing device may be a desktop computer or other suitable computing device which is able to communicate with some of the hardware components in FIG. 50B perhaps wirelessly in which case the computing device may be a tablet.

The computing device 202 generally comprises a processing unit 250, an Analog to Digital Converter (ADC) 256, a data store 252, a display 254 and an input/output interface 258 which may be coupled to various peripheral components such as the laser 204 and the CCD camera 230, or a prepackaged Raman microscope which includes these hardware components. The computing device 202 may also include a power unit (not shown) or be connected to a power source to receive power needed to operate its components.

The processing unit 250 is operatively coupled to the other components of the computing device 202 for controlling various operations and performing certain functions, such as setting or modifying stimulus parameters for the laser 204 (i.e. wavelength and intensity), the data acquisition process (i.e. controlling image acquisition, etc.) and assessing patient samples for various aspects of cancer as described herein.

The processing unit 250 can be any suitable processor, controller or digital signal processor that can provide sufficient processing power depending on the operational requirements of the computing device 202 as is known by those skilled in the art. For example, the processing unit 250 may be a high performance processor. In alternative embodiments, the processing unit 250 may include more than one processor with each processor being configured to perform different dedicated tasks.

The data store 252 includes volatile and non-volatile memory elements such as, but not limited to, one or more of RAM, ROM, one or more hard drives, one or more flash drives or some other suitable data storage elements. The data store 252 may be used to store an operating system and programs as is commonly known by those skilled in the art. For instance, the operating system provides various basic operational processes for the processing unit 252 and the programs include various operational and user programs so that a user can interact with the computing device 202 to perform Raman imaging of a sample and subsequent cancer assessment of the sample, to determine and/or update models (e.g. through training) used in the cancer assessment or a combination of both of these operations.

The data store 252 may also include software code for implementing various components for Raman imaging, model training and various aspects of cancer assessment in accordance with the teachings herein as well as storing values for various operational parameters that are used for Raman imaging. For example, the data store 252 can include programs for implementing an input/output module 252a, a cancer assessment module 252b, a Raman imaging module 252c, and a training module 252d. It should be noted that there may be other embodiments in which the software modules may be organized differently, however, the same functions as described herein are performed.

The input/output module 252a can include program instructions for receiving acquired Raman image data and user control data. The input/output module 252a can also include program instructions for outputting and/or storing raw Raman image data, preprocessed Raman image data, and cancer assessment data.

The cancer assessment module 252b includes program instructions for obtaining and preprocessing Raman spectral data for a patient sample, performing feature extraction on the preprocessed Raman spectral data and providing feature extraction values to various models including classifiers for detecting various cancer characteristics that can be used for providing a cancer assessment for the patient sample based on one of more of the biomarkers described herein. The cancer characteristics may include cancer type, cancer stage, cancer grade, cancer metastasis, potential cancer metastasis or any combination thereof. If the analysis is done based on two or more biomarkers then the results from each biomarker can be combined to provide a cancer assessment with improved accuracy compared to basing the analysis on only one biomarker. The operation of the cancer assessment module 252b is further described with reference to FIG. 50D.

The Raman imaging module 252b is used to control the generation of excitation laser pulses by the laser 204 and recordal of resulting images when the patient sample 217 is on the moveable stage 216. The Raman images may then be directly processed by the cancer assessment module 252b and optionally stored on the data store 252.

The training module 252d includes program instructions using training data to obtain determine the models (e.g. classifiers) used by the cancer assessment module 252b as well as update these models over time as more training data is acquired. The training data may be obtained using the hardware setup of FIG. 50B or it may be obtained using other means and then accessed by the input/output module 252a whenever training is performed. In some embodiments, the training data may be stored in the data store 252. Once the training is performed for the models, the model parameters 252e may be stored in the data store 252. The operation of the training module 252d is described in more detail with respect to FIG. 50E.

The display 254 can be any suitable device for displaying images and various types of information such as an LCD monitor or touchscreen display. The input/output interface 258 can be various ports such as one of more of USB, Firewire, serial and parallel ports, for example, that may be coupled with various peripheral devices used for the input or output of data. Examples of these devices include the laser 204 and CCD camera 230 and may also include, but are not limited to, a keyboard, a mouse, a trackpad, a touch interface, and a printer or any combination thereof. The Analog to Digital Converter 256 may be needed to convert any analog data that is received by the input/output interface 258 into digital data.

Returning back to FIG. 50A, at step 110, the computing device 202 (i.e. a processor thereof) performs processing to analyse the one or more Raman spectra obtained from steps 107, 108. For instance, in one embodiment, at step 110, the one or more obtained Raman spectrum is/are compared to one or more template Raman spectra using correlation analysis, when the processor is executing one of the computer programs.

In at least one embodiment, the correlation analysis of the one or more obtained Raman spectra with the template Raman spectrum includes a comparison of signature peaks of Raman bands of the obtained Raman spectrum with signature peaks of Raman bands of the template Raman spectrum. The template Raman spectrum includes an average spectrum of a plurality of individual Raman spectra from samples of known cancer characteristics.

Raman spectra of plasma/cfDNA/exosomes, for example, contains rich information. This information is typically in the form of signature peaks. For example, the peaks at a particular wavenumber typically represent a type of biomolecule. Some representative Raman assignments are provided here as an example. For instance, a peak at 782 cm⁻¹ is assigned to the ring breathing mode of DNA/RNA bases, a peak at 1445 cm⁻¹ CH₂ is assigned to bending modes of proteins, a peak at 1670 cm⁻¹ is assigned to stretching modes of C═C in lipids, and a peak at 2850 cm⁻¹ is assigned to CH₂ symmetric stretch in lipids. The Raman assignments are well established by known literature. It should be understood that specific signature peaks of the spectra can be selected depending on the application of the data.

In at least one embodiment, the correlation analysis may include use of one or more correlation analysis tools. Some of the tools for performing correlation analysis may include: Pearson's correlation analysis, Spearman correlation test, Heat maps and/or plotting correlation matrix of eigenvalues using artificial neural network. In at least one embodiment, during the correlation analysis, ten measurements from one sample are obtained.

In at least one embodiment, a large number of peaks in any given spectrum may be selected. The number of peaks may be determined by the molecular vibrations in each cell or biomarker. Generally, a minimum of three peaks are considered for every cell or biomarker used. In at least one embodiment, the number of peaks used may be in a range of 3 to 10. In at least one embodiment, there may be overlap of many peaks in the Raman spectra of relatively large macromolecules like proteins, lipids and/or nucleic acids. Thus, many inaccuracies may be introduced into the analysis when the spectra are analyzed qualitatively and certain peaks are assigned to specific biochemical components. These errors are introduced from visual inspection and guessing to determine biochemical components from changes in intensity as there always is a possibility of combinational contribution from several components contributing to one peak. Also, there is a possibility of loss of important information from omitted regions of spectra. To overcome these errors, chemometric methods of multivariate data analysis can be employed, as is described with reference to FIGS. 61C and 61D.

In at least one embodiment, a diagnosis may be made if there is high similarity between the sample Raman spectrum and the template Raman spectrum. For instance, in at least one embodiment, if the correlation analysis of the obtained Raman spectrum (from the subject sample) with the template Raman spectrum demonstrates correlation of 65% or above, a diagnosis may be made based on the underlying cancer characteristics of the template Raman spectrum.

In at least one embodiment, the obtained Raman spectrum/spectra may be compared to more than one template simultaneously to make a multiplex diagnosis. In at least one embodiment, the comparison of the obtained Raman spectrum/spectra to the template(s) may be completed by a computer program. For instance, in at least one embodiment, Python™ and/or Matlab® modules may be used for data comparison and data interpretation.

Referring now to FIG. 50D, shown therein is a flow chart of a method 300 for performing a cancer assessment on a subject sample using Raman spectra obtained therefrom that may be performed by software of the computing device 202 according to at least one example embodiment described herein. In alternative embodiments some aspects of the method 300 may be modified. For example, while the application of four classifiers is shown in FIG. 50D, it is possible that some of the classifiers are not applied on the basis of the underlying biomarkers that are used for performing classification.

At acts 302 and 304, the Raman spectrum of the patient sample are obtained and preprocessed. This may be done using the hardware setup of FIG. 50B or this Raman spectrum may have already been obtained in which case it is accessed from storage and might already have been preprocessed. The preprocessing involves removing any amplitude shifts that are due to the baseline in the Raman spectrum data when this data is first acquired. The baseline may induce uneven amplitude shifts across different wavenumbers and negatively affect the analysis that is done by the cancer assessment module 252b. The baseline correction that is performed during preprocessing at act 304 is known to those skilled in the art.

At act 306, feature extraction is performed on the preprocessed Raman spectrum to obtain values for features that are used by the models in later steps of method 300. The feature extraction results in a reduction in the dimensionality of the data. The feature extraction may be performed using several different techniques. For instance, at step 306, Principal Component Analysis (PCA) may be used as a non-parametric approach that does not need any explicit background model. In principal component analysis, the input Raman spectral data is decomposed and the output that is generated is the principal components. The first principal component can be defined as a direction maximizing the variance. The i^{th} principal component is orthogonal to the first principal component maximizing the variance. In short, principal components are the eigenvectors of the covariance matrix of the input Raman spectral data. One can determine how many principal components are to be used for analysis ignoring the rest of the components. Known techniques may be used to perform the PCA to produce a certain number of principal components such as 10 principal components, for example. In at least one embodiment, described herein, the components that show maximum amount of data are used (e.g. usually three to five components are selected, which represent at least 70% of spectral data). The principal components are the features that are used as inputs to the classifiers. The principal components that are calculated can be further used for solving the problem of classification of one or more components (e.g. extracellular vesicles) that are detected after the sample is on the nanosensor and the Raman image is captured.

Alternatively, at act 306, Multivariate Curve Resolution analysis (MCR) may be used to provide an estimation of contributions of pure ingredients with mixed measurements (for example, a mixture of various types of cfDNA in a plasma sample) providing information on component profiles to generate scientifically meaningful information, which in this case are the features that are then provided to the classifiers. In this way, interpretation of results from complex and large data gathered from Raman spectra may be easily understood.

MCR analysis generally provides more accurate information on the pure components in a mixture compared to the PC analysis. Therefore, by applying it to the extracellular vesicles, one can derive the variation in the pure components of extracellular vesicles. Alternatively, PC analysis is faster than MCR analysis. However, it should be understood that the choice of whether to perform PC analysis or MCR analysis depends on which type of analysis was used in generating/training the classifiers (e.g. classification models). For example, if PC analysis was used to generate/train the cancer type classifier used in act 308 then PC analysis is performed at act 306 and the extracted feature values are input to act 308 meanwhile if MCR analysis was used to generate/train the cancer stage classifier used in act 310 then MCR analysis is performed at act 306 and the extracted feature values are input to act 310. Accordingly, it is possible that both PC and MCR analysis may be performed in act 306 if PC and MCR analysis were each used in determining/training at least one of the classifiers used in acts 308 to 314.

At act 308, the extracted feature values from act 306 are provided as input to a cancer type classifier which provides probabilities for different cancer type classes that are used to detect whether at least one type of cancer is present in the patient sample. This detection result is then provided to act 316 where it is incorporated into the cancer assessment that is performed. It should be noted that if no cancer type is detected at act 310, then there is no need to perform acts 310 to 314.

At act 310, the extracted feature values from act 306 are provided as input to a cancer stage classifier which provides probabilities for different cancer stage classes that are used to detect what the cancer stage is if there is cancer present in the patient sample. This detection result is then provided to act 316 where it is incorporated into the cancer assessment that is performed.

At act 312, the extracted feature values from act 306 are provided as input to a metastasis classifier which provides probabilities for whether the cancer is metastasized or not when there is cancer present in the patient sample. This detection result is then provided to act 316 where it is incorporated into the cancer assessment that is performed.

At act 314, the extracted feature values from act 306 are provided as input to a potential metastasis classifier which provides probabilities for potential cancer metastasis when there is cancer present in the patient sample. This detection result is then provided to act 316 where it is incorporated into the cancer assessment that is performed.

It should be noted that one or more of acts 308 to 314 may be optional in some embodiments.

At act 316, the detection results from acts 308 to 314 which were performed (as some might be optional) are used to provide a cancer assessment, which might be considered to be a cancer diagnosis for the subject based on the analysis of the subject sample. For instance, the cancer assessment, might be that the subject has a certain cancer type, a certain cancer stage, a certain cancer grade, whether the cancer has metastasized and the potential for cancer metastasis to occur. For example, the subject may have breast cancer that is at stage 4 which has metastasized and based on further potential metastasis, the subject may be given a probably of survival of 5 years.

The classification models that are used in acts 308 to 314 can be based on a machine learning model. For example, the machine learning model may be based on Partial Least Square Discriminant Analysis (PLSDA), Support Vector Machine Discriminant Analysis (SVMDA), or an PLSDA or an Artificial Neural Network topology. Alternatively, other types of machine learning techniques might be used such as, but not limited to, Convolutional Neural Networks, and the Random Forrest method, for example.

In an alternative embodiment, the classification models may be based on a deep learning model in which case the feature extraction step of act 306 does not need to be performed since the deep learning model can perform feature extraction while it determines a classification result.

However, it is possible that each of the classifiers used in acts 308 to 314 are based on different types of models (some examples of which were listed previously) that may be obtained using the training method 400 shown in FIG. 50E. In particular, the training method 400 can be used to determine the best classification model to be used for each of the classifiers used in acts 308 to 314. Each of the classification model parameters can be stored in the data store 252. In at least one embodiment, Matlab-based software and/or Python open source libraries may be used for implementing the classifiers used in acts 308 to 314.

Classifiers that are based on PLSDA, determine a probability of a sample belonging to each class along with a classification threshold. This is done by fitting a Gaussian distribution to all calculated class probabilities.

Classifiers that are based on SVMDA, use a supervised method for classification, regression and outlier detection. The SVMDA is an effective tool in high dimensional spaces. Features are separated in different domains and a probability of each sample belonging to a certain class is calculated. These probabilities are used to estimate the most likely class for each sample (e.g. prediction probability).

Classifiers that use an Artificial Neural Network (ANN) employ a sequential model to build a solution to a problem layer by layer. A “hard sigmoid” activation function may be used for each computation node with binary cross entropy to predict the probability of origin of cancer with, an Adam optimizer algorithm, for example. The cancer origin may be used to determine the cancer type, e.g. lung cancer versus breast cancer. The Adam algorithm utilizes a method of adaptive learning rates and obtains learning rates for individual parameters to optimize the ANN. Alternatively, the ANN may be also be useful, for example, in classifiers that are used to obtain information on the trajectory of cancer prognosis. The ANN may be implemented using python's keras, numpy or sklearn functions, for example.

It should be noted that the method 300 can be performed for each biomarker that is used in performing the cancer assessment which may increase the accuracy of the cancer assessment. Each biomarker will have its own set of classification models. For example, if two biomarkers are used, e.g. a first biomarker and a second biomarker, then there will be a first set of classifiers including a first set having a first cancer type classification model, a first cancer stage classification model, a first metastasis classification model and a first metastasis potential classification model that all correspond to the first biomarker that are used to provide a first set of detection results and then a second set having a second cancer type classification model, a second cancer stage classification model, a second metastasis classification model and a second metastasis potential classification model that all correspond to the second biomarker that are used to provide a second set of detection results. The first and second detection results are then combined at act 316 to provide a more accurate cancer assessment. In at least one embodiment, any combination of the features for cell free DNA, immune biomarkers, exosomes and methylation markers may be used as the training dataset. The combination of biomarkers improves the accuracy of the detection process when compared to using single biomarker. The first and second biomarkers for the classification model can be, tumour associated immune cells, cell free DNA, immune biomarkers, exosomes or methylation markers.

Accordingly, in at least one embodiment, the cancer assessment at act 316 can be comprehensive depending on the types of biomarkers that are used for performing the method 300. For example, in at least one embodiment, the cancer assessment may include: cancer location, cancer stage, cancer grade, cancer characteristics such as but not limited to metastatic potential, therapy efficacy and the development of adoptive immunity to cancer. Any of the biomarkers described herein can be used to detect these cancer characteristics although the specificity and accuracy can change depending on the particular biomarker used to detect a given cancer characteristic. In at least one embodiment, the comprehensive cancer analysis may include a prognosis for a cancer patient.

Referring now to FIG. 50E, shown therein is a flowchart of an example embodiment of a method 400 for performing training to obtain models that may be used by the software of the computing device 202 in detecting one or more cancer characteristics from the patient sample. For example, the method 400 can be used for obtaining the best model for each of the classifiers that are used in acts 308 to 314 of the cancer assessment method 300. In alternative embodiments, some aspects of the method 400 may be modified.

Acts 402, and 404 are performed in a similar fashion as acts 302 and 304 of method 300. Act 406 involves using PC analysis or MCR analysis, which can be performed as descried previously. However, only one of PC or MCR analysis is performed based on the type of model that is being generated/trained. This may be determined based on the nature of the underlying biomarker for which the model is used.

It should be noted that acts 402 to 406 are performed on several samples to obtain training data which is then used at act 408 for generating/training the classification model. For example, ten or more known cancer samples may be used for training.

Act 408 then involves using the training data from acts 402 to 406. For each sample, there are several features that are used for the training depending on the model structure. For example, when the model is based on PLSDA, the features can be provided by the MCR analysis in which case the first five MCR components can be used.

At act 410, an accuracy assessment of the generated/trained model may be performed, which might be done by running several classifiers in series by applying preprocessing and feature extraction to Raman spectrum of known cancer samples. The classifier giving the highest classification probability is used to determine the type of cancer of the Raman spectrum. This Raman spectra associated with this type of cancer can be removed and the analysis can be repeated for classifying the remaining cancer types.

At act 412, if a number of classification models have been generated/trained then the most accurate one is selected as the optimal model. For example, the model may be further optimized by performing 2,000 iterations for feature extraction and using the features values for the training dataset. Alternatively, a minimum of 10 spectra may be used in the training data for statistical purposes to ensure reproducibility. Each of these spectra were acquired three times and averaged.

### EXAMPLES

### Example 1—T Cells for Cancer Diagnosis

The immune system primarily comprises of immune cells such as lymphocytes and monocytes in peripheral circulation, out of which T cells and B cells play a pivotal role in immune responses to diseases (Ichimura, T et al., 2016). Among the lymphocytes, T cells are the highest in number. Zhao, W et al. (2021) performed single cell RNA sequencing of 8397 cells from Glioblastoma patient biopsy and generated a sequencing library (Zhao, W et al., 2021). Oncogenes were collected from Cancer Gene Census (CGC), OncoKB, Network of Cancer Genes (NCG), TSGene, IntOGene and Volkan Okur and Wendy K. Chung, 2017. T cells show an elevated expression of oncogenes which confirms the presence of cancer related features that make T cells a potential diagnostic element.

T cells play a crucial role in modulating the cancer microenvironment¹. There are differences in the phenotype of T cells in cancer and noncancer states. For example, differentiation status is essential for T cell exhaustion, antitumor immunity, and pharmacological interventions^{2,3}. Strategies have been developed to produce less differentiated T cells. The morphological features of single T cells can also enable accurate differentiation between cancer and noncancer cells. Therefore, understanding the T cell phenotype is essential for Differentiating between cancer and noncancer Microenvironments.

The immune system predominantly shapes tumor growth, progression, and metastasis. Therefore, the presence of immune cell infiltration within the tumor microenvironment largely determines the prognosis of the patient (Varn, F S et al., 2018). Additionally, findings have shown that immune cells can have both pro-tumor and anti-tumor effects based on the tumor stage (Wellenstein, M D et al., 2018). Distinct cancer types have been associated with a characteristic immune activation state in the tumor microenvironment. For example, studies have shown that a high intratumoral abundance of CD8 T cells in breast cancer (Savas P et al., 2016) correlates with high patient survival.

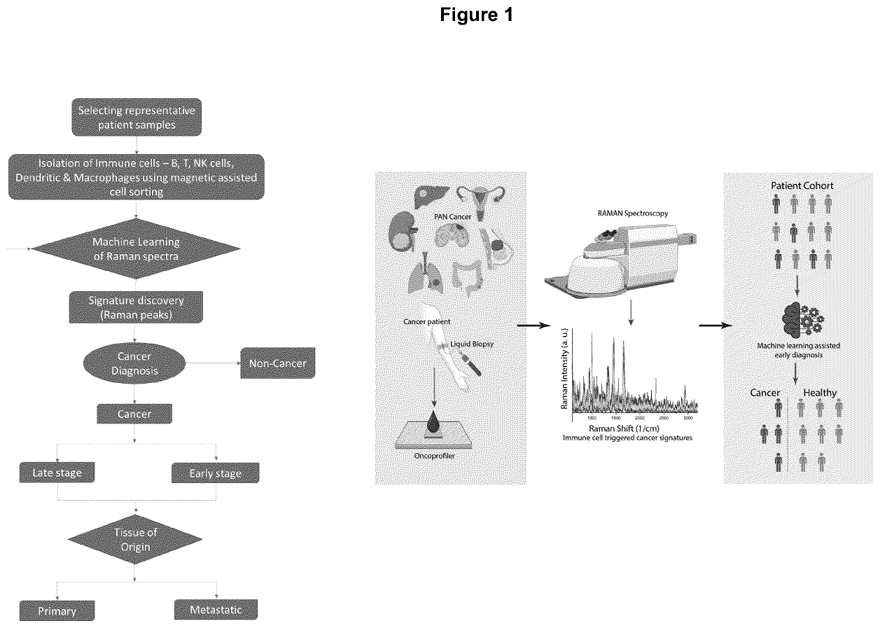

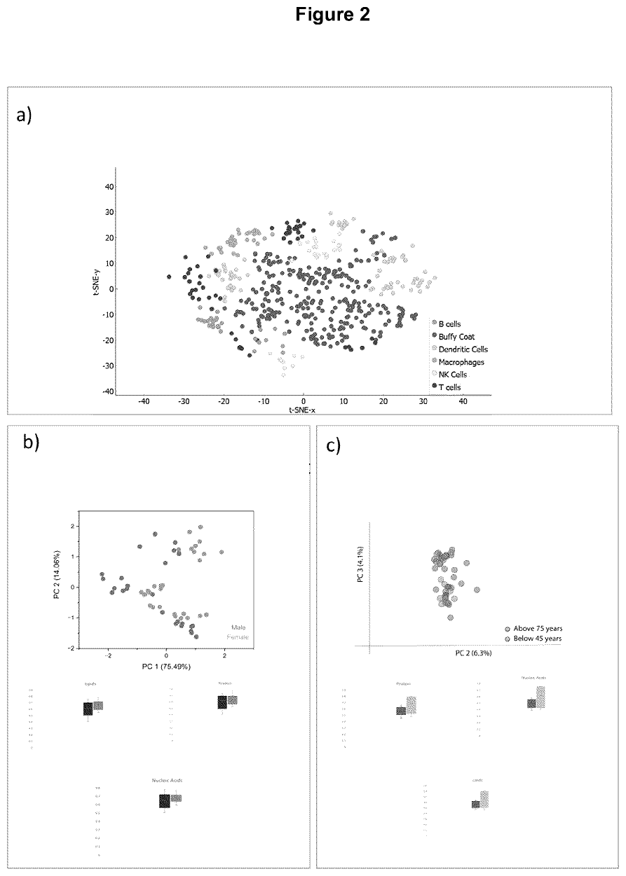

Similarly, the presence of CD4 T cells has been associated with adverse outcomes in bladder cancer and favorable outcomes in lung cancer (Gentles, A J et al., 2015). These clinical insights suggest that immune profile variations are phenotypically and functionally distinct and could be used to characterize cancers based on the molecular subtype. FIG. 1 shows a schematic of an experimental design for obtaining immune cell signatures (a), along with a clinical workflow (b). FIG. 2 shows the viability of tumor associated immune cell-based signatures for comprehensive cancer assessment (a), and further shows that the tumor associated immune signatures show significant overlap between genders (b), and do not vary with age (c). Therefore, distinct immune profile variation is usable for cancer diagnosis.

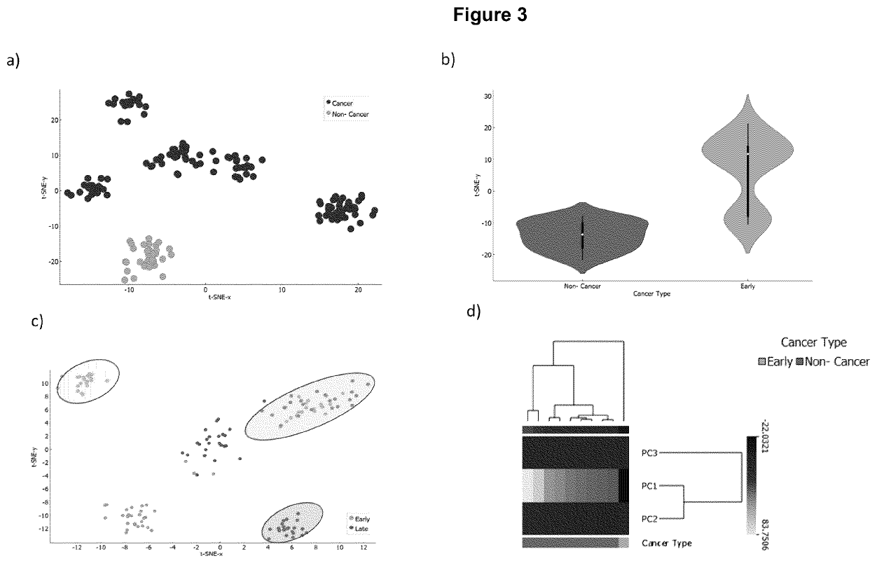

The t-distributed stochastic neighbor embedding (tSNE) analysis observed in FIG. 3, a) shows the variation in a T cell phenotype between cancer and noncancer, showing that the Raman signature of tumor-associated T cells distinctly varies between noncancer and cancer by utilizing tSNE. Tumor-associated T cells were used for comprehensive cancer assessment, including for cancer diagnosis, detection of early-stage cancer, identification of cancer tissue of origin, and cancer metastasis detection. It can be observed in FIG. 3, c) that the signature of tumor-associated T cells also varies between early-stage and late-stage cancers. A phenotype specific to the cancer tissue of origin was also observed. Similarly, the heat map in FIG. 3, d) shows that most of the differences between cancer-associated T cells and non-cancer-associated T cells are contributed using the PC1 corresponding to the peak positions in Table 1. This unique phenotype can be applied to determine the cancer's tissue of origin and the ability to diagnose multiple cancers at early-stage.

TABLE 1
Peak positions corresponding to PC1 contributing to the differences
between cancer associated T cells and non-cancer associated T cells.

| PC1-Peaks | Assignment |
|---|---|
| 676.22 | DNA (G) |
| 854.81 | Glycogen |
| 953.12 | Proline/Valine |
| 1129.6 | Lipid acyl backbone |
| 1339.5 | Tryptophan |
| 1419.8 | CH2 scissoring vibration (lipid band) |

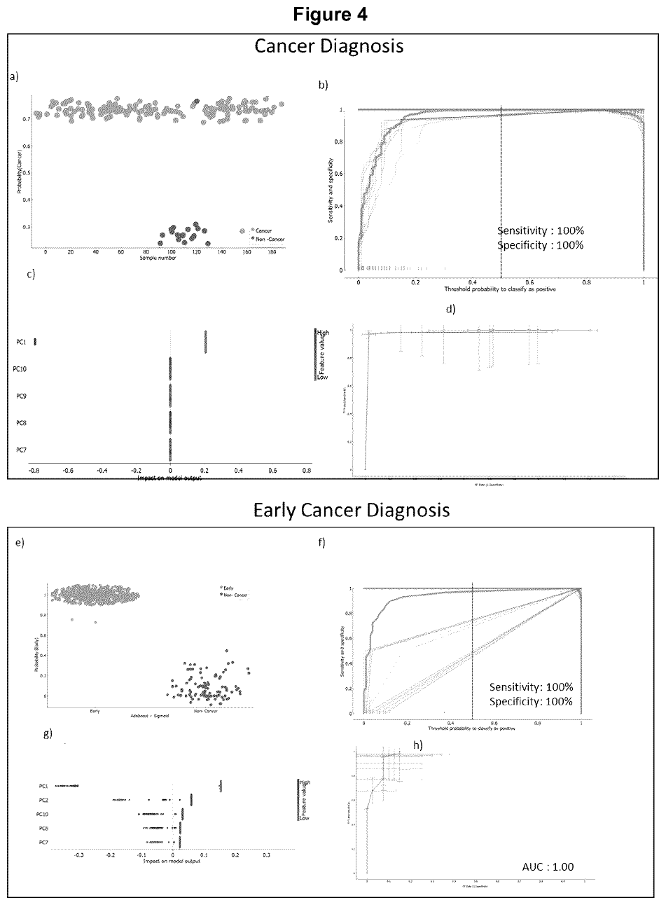

These unique differences between tumor-associated T cells and noncancer T cells were tested for cancer diagnosis, as shown in FIG. 4. FIG. 4, a) shows the scatter plot that showcases the probability of a sample being classified as cancer using this unique tumor-associated T-cell signature. It can be observed from FIG. 4, a) that the classification is accurate, with only one sample being misclassified as cancer, and no cancer sample being misclassified as non-cancer. FIG. 4, b) shows the calibration curve of the machine learning model's performance for cancer diagnosis using the unique signature of tumor-associated T cells identified above. Based on the machine learning model, 100% sensitivity and specificity were achieved. Next, the principal components contributing to accurate cancer diagnosis were analyzed. It can be observed from FIG. 4, c) that the primary principal component, PC1, shows the maximum contribution in assessing the probability of a sample being diagnosed with cancer. Further, to assess the machine learning model's performance, the tradeoff between sensitivity and specificity known as the receiver operating characteristic (ROC) curve was used. It can be observed from FIG. 4, d) that the area under the curve was 1.00, confirming the accurate cancer classification using the tumor-associated T cell signature.

Next, the unique tumor-associated T-cell signature was used to assess the applicability of the signature for early-stage cancer diagnosis, as first indicated in FIG. 3. Diagnosing cancer early is critical because it increases the chances of successful treatment and survival⁴. Further, delayed or inaccessible cancer care can lead to a lower chance of survival, more significant problems associated with treatment, and higher care costs. Further studies have shown that T cells play a crucial role in directly and indirectly fighting cancer. Killer T cells can kill cancer cells directly, while helper T cells organize and orchestrate the fight against cancer⁵. So, defining the precise phenotype and function of the tumor-specific T cell population is a central goal of tumor immunology with important implications for cancer diagnosis, patient prognosis, and therapy in the context of early and metastatic cancer⁶. Furthermore, recent studies have shown that genetic variations in cancer immune response pathways can impact outcomes. They can serve as predictors for treatment efficacy in early-stage lung cancer patients⁷.

FIG. 4, e) shows the scatter plot that showcases the probability of a sample being classified as early-stage cancer using a tumor-associated T-cell signature. It can be observed that the tumor-associated T-cell signature provides an accurate classification of early-stage cancer and can distinguish between early-stage and noncancer samples with 100% sensitivity and specificity (FIG. 4, f). In analyzing the principal components contributing to the accurate diagnosis of early-stage cancer, PC1 and PC2 were the primary factors for early-stage cancer diagnosis (FIG. 4, g). Next, the model's accuracy was assessed using the ROC Curve, where the observed area under the curve was of 1.00 (FIG. 4, h).

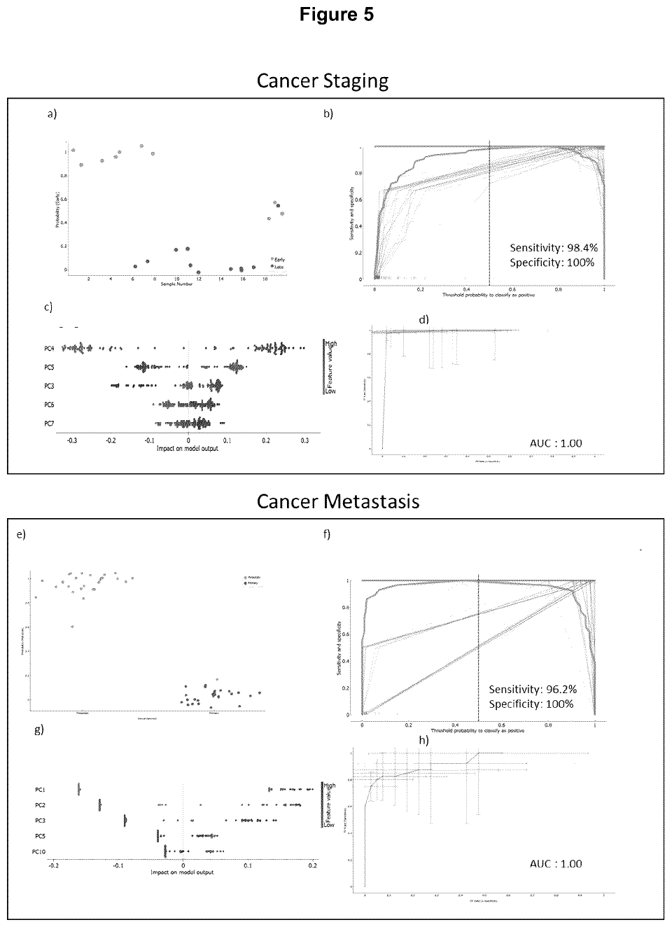

FIG. 5, a) shows the scatter plot that showcases the probability of a sample being classified based on cancer-stage using a tumor-associated T-cell signature. It can be observed that the tumor-associated T-cell signature provides an accurate classification of cancer stage and can distinguish between early-stage and late-stage samples with 100% sensitivity and specificity (FIG. 5, b). In analyzing the principal components contributing to the accurate diagnosis of early-stage cancer versus late-stage cancer, PC4 and PC5 were identified as the primary factors for early-stage vs late-stage cancer diagnosis (FIG. 5, c). Next, the model's accuracy was analyzed using the ROC Curve, where the area under the curve was of 1.00 (FIG. 5, d). The prominent peaks in PC4 and PC5 are 1002 cm⁻¹, 1090 cm⁻¹, 1324 cm⁻¹, 1449 cm⁻¹ and 1659 cm⁻¹, which correspond to amino acids from proteins, nucleic acids, lipids and RNA respectively.

Further, the unique tumor associated T-cell signatures can be used to distinguish primary from metastatic cancer. FIG. 5, e) shows a scatterplot demonstrating the probability that a sample is classified as metastatic/potential to metastasize. It can be observed that the tumor-associated T-cell signature provides an accurate classification of metastasis and can distinguish between metastatic and primary samples with 96.2% sensitivity and 100% specificity (FIG. 5, f). In analyzing the principal components contributing to the accurate diagnosis of metastatic cancer versus primary cancer, PC1 and PC2 were identified as the primary factors (FIG. 5, g). Next, the model's accuracy was analyzed using the ROC Curve, where the area under the curve was of 1.00 (FIG. 5, h).

To summarize, the signatures from cancer associated T cells formed a cluster separate from healthy T cells. The peak differences are predominantly due to shifts in the protein, DNA, lipid and RNA peaks. The spectral data was then analyzed by performing t-distributed stochastic neighbor embedding (tSNE) classification. The T cells formed two distinct clustered groups (FIG. 3, a), implying that they display different phenotypes and cellular compositions when from cancer samples versus from noncancer samples. The violin plot in FIG. 3, b) shows the significant differences exhibited by the spectral data of the T cells associated with early cancer and non-cancer.

Similarly, FIG. 2, a) shows the scatter plot of the tSNE analysis of the SERS data of immune cells associated with cancer and buffy coat from patient blood. The clustering was uniform and widespread, implying that the immune cells express diagnostic features that are demonstrated in FIG. 3, a) for T cells. Therefore, the immune cells in patient circulation exhibit similarity, indicating that immune cells in circulation reflect the entire spectrum of cancer characteristics, and can be assessed for effective cancer diagnosis from liquid biopsy.

### Methods

T cells were isolated from patient tumor by mincing the tumor and digesting the tissue to release the tumor infiltrated T cells according to the following protocol. The surgically resected tumors were minced using a sterile 10 blade and enzymatically digested using collagenase and hyaluronidase at 37° C. The obtained suspension was filtered repeatedly through a 100 μm filter. The suspension was then treated with RBC lysis buffer and incubated at 25° C. for 5 minutes followed by centrifugation and washing using 1×PBS. T cells were isolated from the single cell suspension by following a magnetic separation method.

### Example 2—Tumor-Associated NK Cells for Cancer Diagnosis

### Methods

### Clinical Sample Acquisition

This study was conducted in accordance with Ryerson Ethics Board of Ryerson University (REB 2020-275). Informed written consent and blood samples from cancer patients were obtained by Ontario Tumor Board (OTB). The buffy coat was extracted by density gradient centrifugation.

### Cell Culture

The primary NK cells (Stem Cell Technologies) and NK-92 cell line (derived from non-Hodgkin lymphoma) were obtained from American Tissue Type Culture Collection (ATCC, USA) and maintained in Alpha minimum essential medium w/o nucleosides with 0.2 mM myo-inositol, 0.1 mM 2-mercaptoethanol, 0.02 mM folic acid, 12.5% horse serum and 12.5% fetal bovine serum. To maintain sufficient proliferation, IL-2 was added at 150 IU/ml. Cancer cell lines were similarly obtained from ATCC for breast adenocarcinoma (MDA-MB231), non-small lung carcinoma (H69 AR) and colon adenocarcinoma (colo 205). Breast cancer cell line was maintained with DMEM with 10% FBS. The lung and colon cancer cell lines were maintained in RPMI 1640 with 10% FBS. All cells were cultured at 37 C in 5% C02 atmosphere.

### Co Culture Assays

NK-92 cells were co-cultured with three cancer cell lines using transwell apparatus (0.4 μm pore size, corning, Lowell, MA). NK-92 cells were seeded in the upper insert of a 24 well transwell plate. The cancer cell lines were seeded in three separate lower chambers. Cancer stem cells (CSCs) were grown separately prior to the assay in a serum free media using ultralow low attachment plates. Tumor spheroids were verified in microscope and seeded in a set of other low chambers. After 24 hours of co-culture, the NK-92 cells were centrifuged and re-suspended in water for SERS analysis. NK cells grown separately without co-culture with cancer cell lines were used as control.

### RT-qPCR

RNA was extracted from the cell pellets by the Trizol/chloroform (Invitrogen) extraction method and resuspended in diethylpyrocarbonate (DEPC)-treated water. RNA concentrations and ratios were determined photometrically (Nanodrop). The expression levels of BCL-2 were assessed by RT-qPCR. RT-qPCR reactions were performed on 96-well plates (Micro Amp® Fast Optical 96-well reaction plate with barcode; ABI, Foster City, CA, USA). The relative expression levels of target genes were expressed as fold-change against housekeeping gene GAPDH. Statistical analysis (one-way ANOVA) were done using Prism Graphpad (v.9) compare the gene expression levels.

### Raman Data Acquisition

5 μl of buffy coat (blood) and 10 μl of cultured NK-92 cells were dropped on the OncoImmune probe platform. After 1 minute (to allow trapping of NK-92 cells in the sensor mesh), Raman spectral scanning was done at 785 nm wavelength. A minimum of 10 spectra and 3 acquisitions were made with laser power at 5 W. The spectra were collectively saved and processed using Spectragryph software.

### Data Analysis

Partial least squares regression analysis was done after doing multivariate analysis. The NK-92 cell culture data was given as training data and the spectra from buffy coat was given as validation data. PLSDA analysis was done using PLS Toolbox software. For the localization of cancer, data from buffy coat of known patient samples were used to train the algorithm. The statistical analysis for all the results is indicated in the figure legends. All data are represented as mean±S.D. For SERS analysis two tailed student t test was performed. P<0.05 was considered statistically significant. Experimental results were done at least three times, unless stated otherwise in figure legends.

### Results

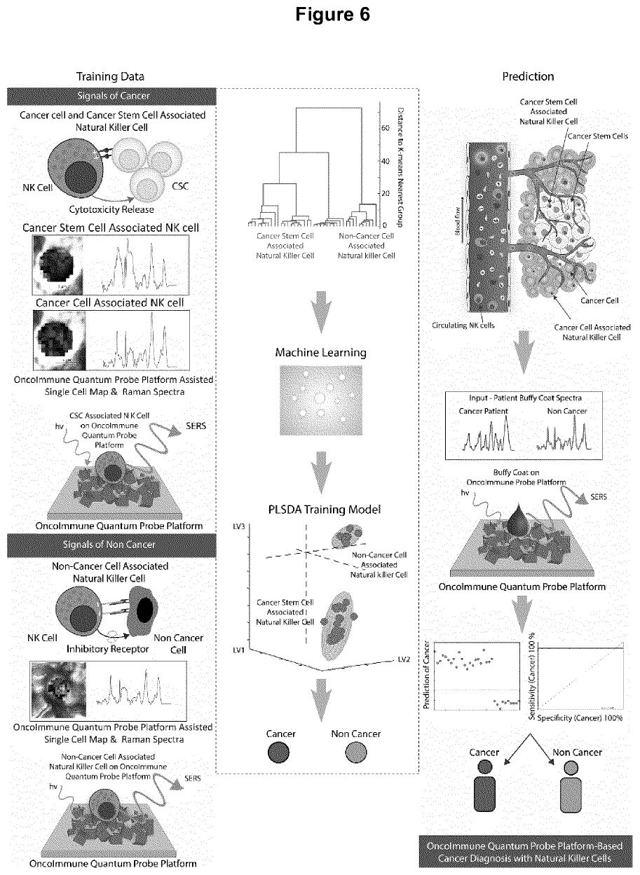

This study demonstrates that molecular probing of natural killer (NK) cells has the potential to provide diagnostic information for cancer patients. The presence of cancer stem cells (CSCs) was detected through changes in NK cell expression, as CSCs resist antiproliferative therapies and have the ability to repopulate bulk tumor (Ames E, et al., 2015). It is essential to detect the presence of CSCs not only for cancer diagnosis but also for patient care management. To detect the presence of CSCs, NK cells were selected for a number of reasons. NK cells, the critical part of innate immune system, are the first line of defense against cancer and are responsible for cancer immune surveillance (Myers J A and Miller J S, 2021). Additionally, NK cells do not require any prior sensitization to recognize tumors (Tallerico R et al., 2013). Moreover, amongst all immune cells, only NK cells demonstrate preferential cytotoxicity towards CSCs (Butler K T, et al., 2018). Although CSCs are able escape other immune cells, CSCs cannot escape NK cell surveillance and demonstrate vulnerability towards NK cells (Luna J I, et al., 2017). Therefore, the presence of CSCs may naturally activate NK cells with signature molecular changes, enabling identification of the presence of CSCs and hence the presence of cancer. FIG. 6 illustrates this diagnostic approach. NK cells were co-cultured with cancer cells as well as CSCs. This led to NK cells exhibiting three phenotypes based on cell-specific association. Consistent with this idea, naïve NK cell spectra, cancer associated NK cell spectra and CSC associated NK cell spectra were obtained from cell culture. The three phenotypes form the basis for distinction of cancer diagnosis in this study. Analysis of SERS spectra of human blood samples based on the similarity to the SERS spectra of NK cell activity using a simple machine learning algorithm was undertaken. The Raman signals of NK cell interaction with cancer cells and CSCs was predicted to be detectable from patient blood. Thus, NK cells were first co-cultured with cancer cells, CSCs, and non-cancer cells and SERS signals were collected using SERS functionalized OncoImmune Probe Platform.

In this study, machine learning (ML), a subfield of artificial intelligence that has evolved rapidly in recent years, was adopted for diagnostic prediction. Unlike conventional techniques, ML techniques have capabilities for addressing complex problems involving massive combinatorial spaces or nonlinear processes without incurring massive computational costs (Guo S., et al., 2021). The use of ML was here explored by adopting an ML approach for cancer diagnosis, to address the complex molecular fingerprinting of tumor-associated NK cells for prediction of cancer. ML was chosen as ML tools have consistently generated, tested, and refined scientific models (Meza Ramirez C A, et al., 2021; Sheetz K E and Squier J, 2009; Wong D M, et al., 2010). This family of statistics-based methods that can make predictions of properties of molecules and materials without invoking computationally demanding electronic structure calculations has the potential to accelerate a variety of applications in chemical and molecular sciences, including Raman spectroscopy. The spectral dataset from co-cultures was used to train the machine learning model with binary classification (cancer & non-cancer). The supervised model successfully classified the co-culture data into two clusters. Human blood samples from cancer patients and healthy controls were also classified through this model.

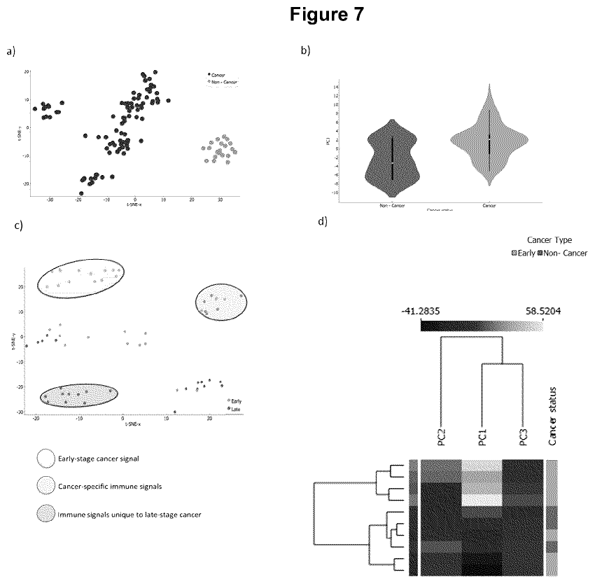

FIG. 7, a) represents the tSNE analysis-based distinction of normal NK cells and tumor associated NK cells. PCA analysis revealed that the Raman signature of tumor associated NK cell shows characteristic bands from carbohydrates, proteins and lipids. Noticeable peaks are phenylalanine band at 1003 cm⁻¹, CH deformation at 1450 cm⁻¹ and amide I at 1661 cm⁻¹. However, the most distinct bands typical of lymphocytes are seen at 1522 cm⁻¹ and 1158 cm⁻¹ belonging to carotenoids (El-Said W A, et al., 2011). Carotenoids are robust Raman scatterers and have characteristic Raman spectra. The presence of carotenoids in NK cells is an indication of cytotoxicity and cell surface activation. Other spectral peaks originate from enzymes in the dense granules within the cytoplasm (Shen Y, et al., 2021).

Ultrasensitive Detection with OncoImmune Sensor Assisted CNKP

Next, the ability of OncoImmune sensor to generate circulating NK cell profile was analyzed. Surface-enhanced Raman scattering (SERS) was adopted for ultrasensitive detection. SERS-based methods, very useful for monitoring intracellular proteins and other macromolecules such as lipids and nucleic acids enabled intensive analysis of cellular biochemical composition (Movasaghi Z et al., 2007). SERS-based molecular fingerprints at the sub-cellular level will allow real-time information in a non-destructive way (López-Soto A, et al., 2017). This marker-free approach will be extremely useful for the analysis of minute intracellular changes (Xu Y, et al., 2020).

NK cells that are completely devoid of any tumor cell association showed characteristic Raman spectra with characteristic bands from carbohydrates, proteins, and lipids. This profile can be correlated to the NK cells in circulation. The band at CH deformation at 1450 cm⁻¹ and amide I at 1662 cm⁻¹, amide II band at 1555 cm⁻¹ and amide Ill band at 1339 cm⁻¹. Other spectral features are disulphide bonds (S—S) between 500-550 cm⁻¹ and aromatic amino acids at 1004 cm⁻¹ (from phenylalanine), 830 cm⁻¹ and 854 cm⁻¹ (from tyrosine doublets), 1340 cm⁻¹ of tryptophan (Jiang S, et al., 1996-1997).

### Evaluation of Cancer Stem Cell Associated NK Cell for Cancer Diagnosis

CSC-associated NK cells were then compared. Major changes in the metabolites in NK-CSCs included significantly decreased disulphide stretching proteins, relative increase in quantity of nucleic acids, decrease in tyrosine and phenylalanine, increase in glycogen and fatty acids and lipids, increase in carotenoids, phospholipids, cytosine, decrease in tryptophan corresponding to 521 cm⁻¹, 787 cm⁻¹, 854 cm-1-¹, 1000 cm⁻¹, 1048 cm⁻¹, 1137 cm⁻¹, 1168 cm⁻¹, 1268 cm⁻¹, 1509 cm⁻¹, 1339 cm⁻¹. The decrease in the peak at 520 cm⁻¹ of NK cell in association with cancer stem cells indicate that NK-CSCs have down-regulated their Killer immunoglobulin receptor (KIR) expression, suggesting a possible decrease in major histocompatibility complex (MHC) expression by CSCs. This paves the way for increased cytotoxicity. The loss or decreased expression in CSCs is correlated with better clinical outcomes and a promising strategy to reverse the immune escape (Kornberg M D, 2020). The most distinct bands typical of lymphocytes are seen at 1522 cm⁻¹ and 1158 cm⁻¹ belonging to carotenoids (El-Said W A, et al., 2011). As mentioned earlier, carotenoids are robust Raman scatterers and have characteristic Raman spectra. Dramatic changes in the profile of CSC-associated NK cells indicated the suitability of CSC associated NK cell for further analysis. One significant feature of this association is that unlike cancer cells, CSCs are unable to escape immune detection with NK cell and their association readily reflects the tumor profile. NK cells have a unique affinity for CSC as shown by many studies where NK cells lyse and kill CSC population in mice. The observed spectra show the immune cell stimulated/inhibited status of the cells. Several markers that are indicative of lymphocyte activation were identified. The peak at 521 cm⁻¹ is a disulphide band suggestive of the formation of immunoglobulins (Schafer J R, et al., 2019). The disulphide bonds are characteristic in Raman spectra and appear distinct and separated from other peaks, and help in the conformation of protein. NK cells associated with CSCs exhibit killer-cell immunoglobulin-like receptors (KIRs) on their surface, which is involved in the education of NK cells.

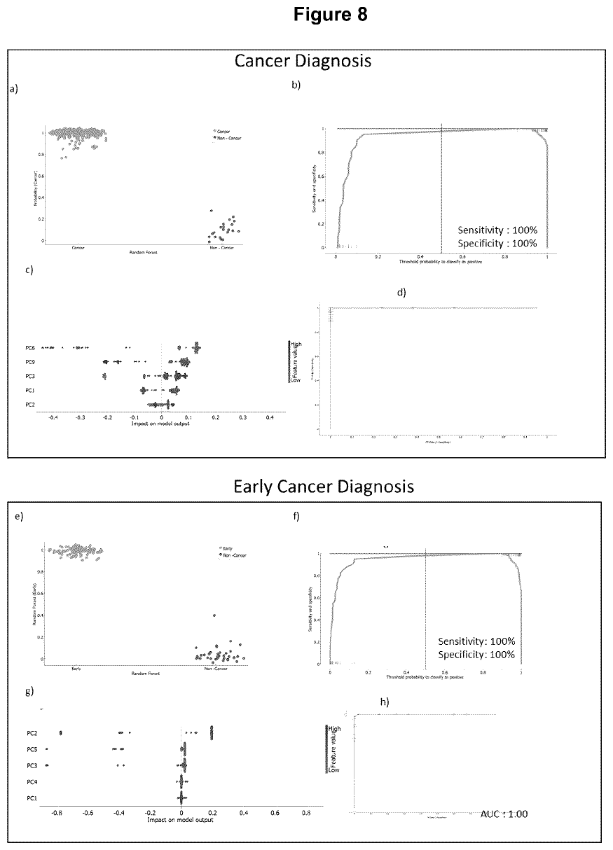

These unique differences between tumor-associated NK cells and noncancer NK cells were used for cancer diagnosis, as shown in FIG. 8. FIG. 8, a) shows the scatter plot that showcases the probability of a sample being classified as cancer using this unique tumor-associated NK-cell signature. FIG. 8, b) shows the calibration curve of the machine learning model's performance for cancer diagnosis using the unique signature of tumor-associated NK cells. Based on the machine learning model, 100% sensitivity and specificity were achieved. Next, the principal components contributing to accurate cancer diagnosis were analyzed. It can be observed from FIG. 8, c) that the primary principal component, PC6, shows the maximum contribution in assessing the probability of a sample being diagnosed with cancer. Further, to assess the machine learning model's performance, the ROC curve was evaluated. It can be observed from FIG. 8, d) that the area under the curve was 1.00, confirming accurate cancer classification using tumor-associated NK cell signature.

Next, the unique Tumor-associated NK-cell signature was used to assess the applicability of the signature for early-stage cancer diagnosis. Studies have shown that NK cells can potentially detect early cancer. In preclinical studies, NK cells were shown to indicate survival and therapeutic response in different types of cancer, as detected by immunohistochemistry, immunofluorescence, or flow cytometry using different surface and functional markers⁸. Molecular probing of NK cells has the potential to provide diagnostic information for cancer patients, and the diagnostic value of NK cell activity is significant. Hence, it can be concluded that NK cells' specific contributions to early-stage cancer are observed in the Raman spectroscopic signature of early-stage and late-stage cancer, as seen in FIG. 7, c).

FIG. 8, e) shows the scatter plot that showcases the probability of a sample being classified as early-stage cancer using a tumor-associated NK-cell signature. It can be observed that the tumor-associated NK-cell signature provides an accurate classification of early-stage cancer and can distinguish between early-stage and noncancer samples with 100% sensitivity and specificity (FIG. 8, f). In analyzing the principal components contributing to the accurate diagnosis of early-stage cancer, PC2 and PC5 were observed as the primary factors for early-stage cancer diagnosis (FIG. 8, g). Next, the model's accuracy was analyzed using the ROC Curve, where the area under the curve was of 1.00 (FIG. 8, h).

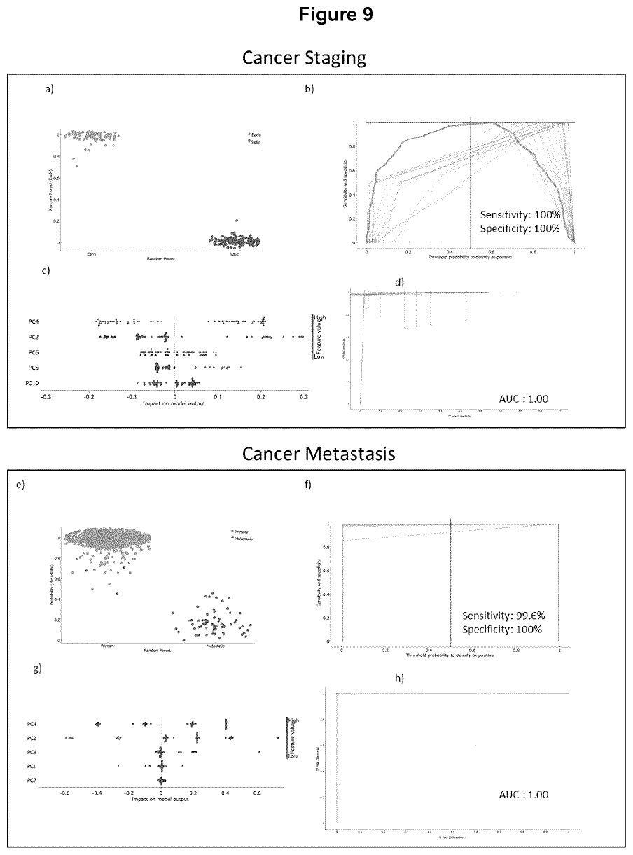

FIG. 9, a) shows the scatter plot that showcases the probability of a sample being classified based on cancer-stage using a tumor-associated NK-cell signature. It can be observed that the tumor-associated NK-cell signature provides an accurate classification of cancer stage and can distinguish between early-stage and late-stage samples with 100% sensitivity and specificity (FIG. 9, b). In analyzing the principal components contributing to the accurate diagnosis of early-stage cancer, PC4 and PC2 were observed as the primary factors for early-stage cancer diagnosis (FIG. 9, c). Next, the model's accuracy was analyzed using the ROC Curve, where the area under the curve was of 1.00 (FIG. 9, d).

NK cells can potentially be used for the detection of cancer metastasis. NK cells serve a critical role in the control of metastasis, and they are shown to preferentially control monoclonal metastases derived from single circulating tumor cells rather than polyclonal metastases derived from cell clusters⁴. Hence, the unique tumor associated NK cell signature was used for detection of cancer metastasis. FIG. 9, e) shows the scatter plot that showcases the probability that a sample is metastatic using a tumor-associated NK-cell signature. It can be observed that the tumor-associated NK-cell signature provides an accurate classification of cancer metastasis and can distinguish between primary and metastatic samples with 100% sensitivity and specificity (FIG. 9, f). In analyzing the principal components contributing to the accurate diagnosis of early-stage cancer, PC4 and PC2 were observed as the primary factors for detecting metastasis (FIG. 9, g). Next, the model's accuracy was analyzed using the ROC Curve, where the area under the curve was of 1.00 (FIG. 9, h), thereby indicating that NK cells can be used to detect cancer metastasis.

### Example 3—Tumor Associated Macrophages for Cancer Diagnosis

Macrophages are essential in the antitumor activity of chemotherapy, radiotherapy, and monoclonal antibodies¹⁵. Tumor-associated macrophages (TAMs) play an essential role in the development of tumors, modulation of neo-angiogenesis, immune suppression, and metastasis¹⁶. The high ratio of macrophages in cancers has been thought to be a mechanism involved in anticancer surveillance¹⁷. Macrophages can facilitate tumor death by promoting cytotoxicity, and targeting macrophages in cancer is a potential therapeutic approach¹⁸. As macrophages are involved in various aspects of cancer development and progression, it is crucial to develop strategies to target them for cancer diagnosis and treatment¹⁹.

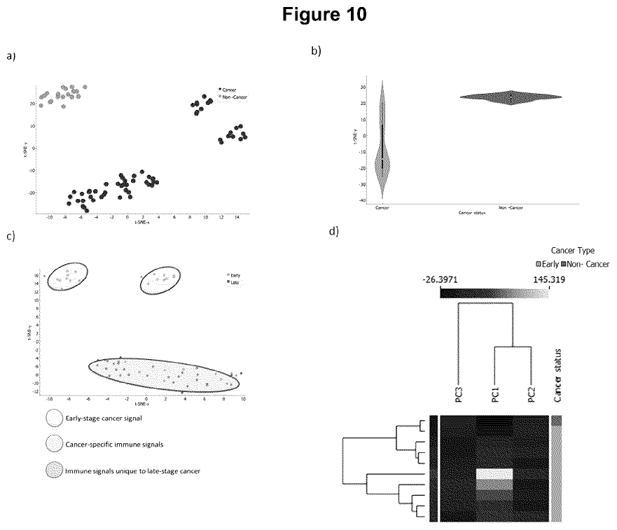

The tSNE analysis observed in FIG. 10, a) shows the variation in a macrophage phenotype between cancer and noncancer, showing that the Raman signature of tumor-associated macrophages distinctly varies between noncancer and cancer. By utilizing this distinct variation, tumor-associated macrophage signatures were used for comprehensive cancer assessment, including cancer diagnosis, identification of early-stage cancer and of cancer tissue of origin, and for cancer metastasis detection. It can be observed in FIG. 10, c) that the signature of Tumor-associated macrophages also varies between early-stage and late-stage cancers. A phenotype-specific to the cancer tissue of origin was also observed. Similarly, the heat map in FIG. 10, d) shows that most of the differences between cancer-associated macrophages and non-cancer-associated macrophages are contributed using PC1 corresponding to the peak positions presented in Table 2.

TABLE 2
The peak Raman shift positions of differences between cancer-
associated macrophages and non-cancer-associated macrophages.

| Raman Shift (1/cm) | Assignment |
|---|---|
| 677.92 | Ring breathing modes in the DNA bases |
| 850.46 | single bond stretching vibrations for the amino acids and valine |
| 1130.5 | Phospholipid structural changes |
| 1341.3 | Nucleic acid mode |
| 1420.3 | CH deformation (DNA/RNA & proteins & lipids & carbohydrates) |
| 1557.4 | Tryptophan |
| 1658.7 | Amide I (a-helix) |

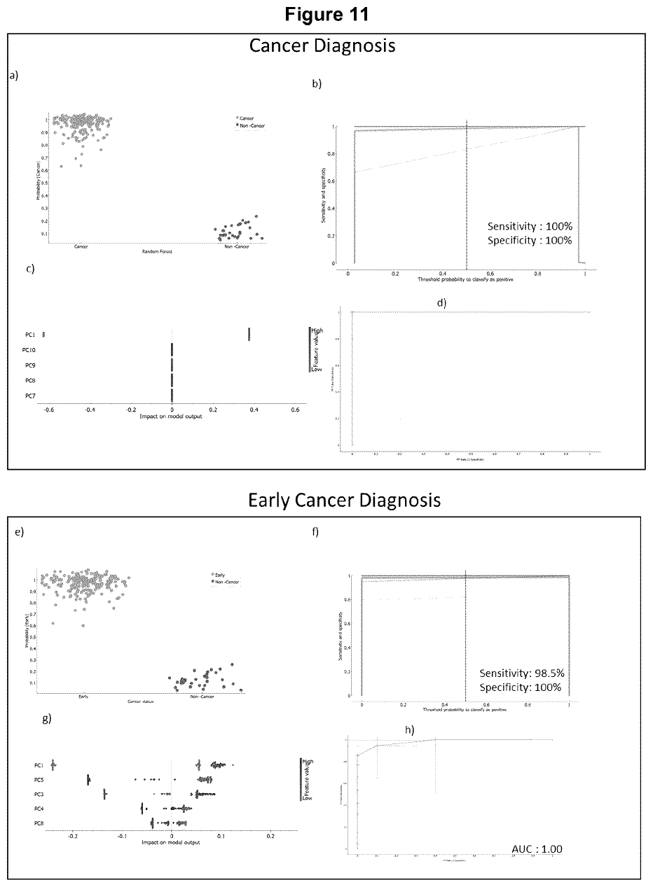

These unique differences between Tumor-associated macrophage cells and noncancer macrophages were used for cancer diagnosis, as shown in FIG. 11. FIG. 11, a) shows the scatter plot that showcases the probability of a sample being classified as cancer using this unique tumor-associated macrophage signature. FIG. 11, b) shows the calibration curve of the machine learning model's performance for cancer diagnosis using the unique signature of tumor-associated macrophage. Based on the machine learning model, 100% sensitivity and specificity were achieved (FIG. 11, b). Next, the principal components contributing to accurate cancer diagnosis were analyzed. It can be observed from FIG. 11, c) that the primary principal component, PC1, shows the maximum contribution in assessing the probability of a sample being diagnosed with cancer. Further, to assess the machine learning model's performance, the ROC curve was analyzed. It can be observed from FIG. 11, d) that the area under the curve was 1.00, confirming the accurate cancer classification using tumor-associated macrophage cell signatures.

Next, the unique tumor-associated macrophage cell signature was used to assess the applicability of the signature for early-stage cancer diagnosis, as initially indicated in FIG. 10, c). Macrophages play diverse roles in cancer development, ranging from antitumor activity in early progression stages to tumor-promoting roles in established cancer¹⁶. Tumor-associated macrophages (TAMs) are one of the important cellular components of the tumor microenvironment and are critical for tumor growth. Depending on their activation status, macrophages can exert a dual influence on tumorigenesis by either promoting or inhibiting cancer development²⁰. In early stages of cancer, macrophages have been thought to be involved in anticancer surveillance and can have antitumor activity¹⁷. However, several studies have shown that macrophages might act as pro-tumor in oncogenesis and neoplastic development by promoting tumor growth and metastasis²⁰. A higher density of M2-type macrophages is associated with high tumor cell proliferation, vascularity, immune suppression, drug resistance, induced histological malignancy, and poor clinical prognosis²¹. Therefore, macrophages have a complex role in cancer development, and their potential use for cancer diagnosis remains an area of active research.

FIG. 11, e) shows the scatter plot that showcases the probability of a sample being classified as early-stage cancer using a tumor-associated macrophage signature. It can be observed that the tumor-associated macrophage cell signature provides an accurate classification of early-stage cancer and can distinguish between early-stage and noncancer samples with 98.5% sensitivity and 100% specificity (FIG. 11, f). In analyzing the principal components contributing to the accurate diagnosis of early-stage cancer, PC1 and PC5 were identified as the primary factors for early-stage cancer diagnosis (FIG. 11, g). Next, the model's accuracy was analyzed using the ROC Curve, where the area under the curve was of 1.00 (FIG. 11, h).

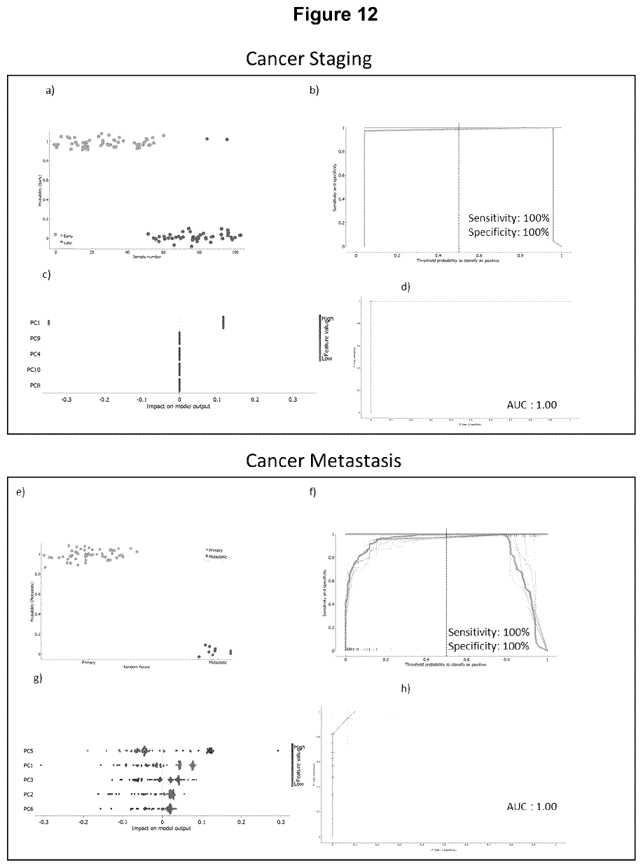

FIG. 12, a) shows the scatter plot that showcases the probability of a sample being classified based on cancer-stage using a tumor-associated macrophage signature. It can be observed that the tumor-associated macrophage cell signature provides an accurate classification of cancer stage and can distinguish between early-stage and late-stage samples with 100% sensitivity and specificity (FIG. 12, b). In analyzing the principal components contributing to the accurate diagnosis of early-stage cancer, PC1 and PC9 were identified as the primary factors for early-stage cancer diagnosis (FIG. 12, c). Next, the model's accuracy was analyzed using the ROC Curve, where the area under the curve was of 1.00 (FIG. 12, d).

Macrophages cells can potentially be used for the detection of cancer metastasis. Tumor-associated macrophages (TAMs) are one of the cellular components of the tumor microenvironment and play an critical role in the development of tumors, and metastasis¹⁸. Macrophages can also induce invasiveness and metastasis, making them a potential target for cancer immunotherapies²⁴. FIG. 12, e) shows the scatter plot that showcases the probability that a sample is metastatic using a tumor-associated macrophages-cell signature. It can be observed that the tumor-associated macrophages-cell signature provides an accurate classification of cancer metastasis and can distinguish between primary and metastatic samples with 100% sensitivity and specificity (FIG. 12, f). In analyzing the principal components contributing to the accurate diagnosis of early-stage cancer, PC5 and PC1 were identified as the primary factors for detecting metastasis (FIG. 12, g). Next, the model's accuracy was analyzed using the ROC Curve, where the area under the curve was of 1.00 (FIG. 12, g).

### Example 4—Tumor Associated Dendritic Cells for Cancer Diagnosis

Dendritic cells have been studied for their potential in cancer diagnosis. Dendritic cells (DCs) are a diverse group of specialized antigen-presenting cells with key roles in initiating and regulating innate and adaptive immune responses²⁵. DCs are specialized in antigen recognition and presentation and play a central role in the initiation of antigen-specific immunity as well as tolerance²⁶. Despite their high potential in promoting antitumor responses, tumor-associated DCs are largely defective in their functional activity and can contribute to immune suppression in cancer²⁷. The potent ability of DCs to initiate and regulate adaptive immune responses underpins the successful generation of anti-tumor immune responses^{26,28}. DCs are the most efficient, potent, and professional antigen-presenting cells of the immune system, inducing and dispersing a primary immune response by activating T cells²⁹. Hence, use of the SERS technique was employed to discover the unique signature of tumor-associated dendritic cells and apply it to cancer diagnosis.

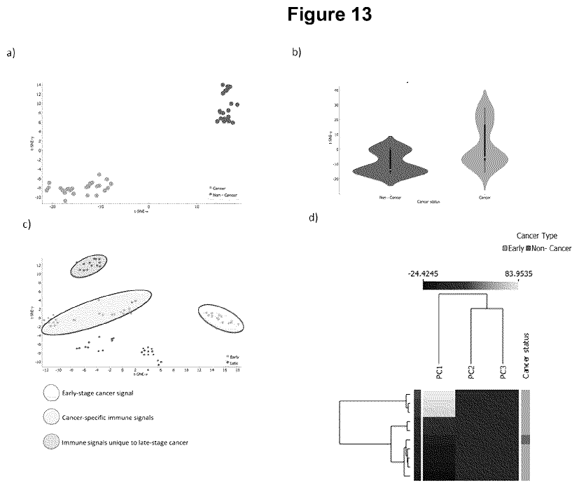

The tSNE analysis observed in FIG. 13, a) shows the variation in a dendritic cell phenotype between cancer and noncancer, showing that The Raman signature of tumor-associated dendritic cells distinctly varies between noncancer and cancer. By utilizing this distinct variation, tumor-associated dendritic cells were used for comprehensive cancer assessment, including cancer diagnosis, detection of early-stage cancer and cancer tissue of origin, and for cancer metastasis detection. It can be observed in FIG. 13, c) that the signature of tumor-associated dendritic cells also varies between early-stage and late-stage cancers. A phenotype specific to the cancer tissue of origin was also observed. Similarly, the heat map in FIG. 13, d) shows that most of the differences between cancer-associated dendritic cells and non-cancer-associated dendritic cells are contributed using the PC1 corresponding to the peak positions presented in Table 3.

TABLE 3
The peak Raman shift positions of differences between cancer-associated
dendritic cells and non-cancer-associated dendritic cells.

| Raman Shift (1/cm) | Assignment |
|---|---|
| 677.07 | Ring breathing modes in the DNA bases |
| 851.51 | single bond stretching vibrations for the amino acids and valine |
| 951.48 | vs(CH3) of proteins (a-helix) |
| 1130.4 | Phospholipid structural changes |
| 1339.9 | C—C stretch of phenyl (1) and C3—C3 stretch and C5—O5 bend |
| | stretch CHa in-plane bend |
| 1419.7 | CH2 scissoring vibration (lipid band) |

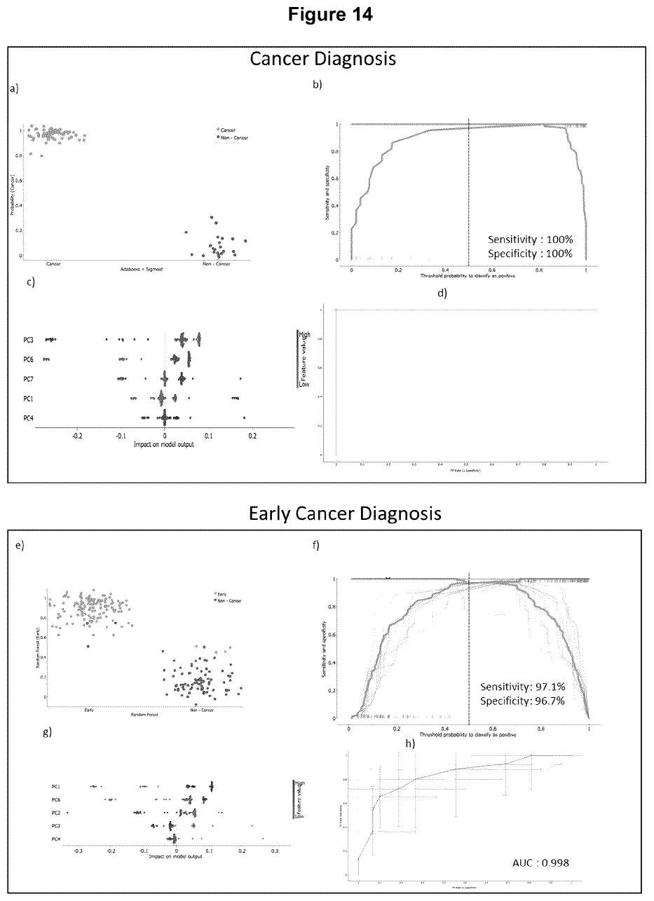

These unique differences between tumor-associated dendritic cells and noncancer dendritic cells were tested cancer diagnosis, as shown in FIG. 14. FIG. 14, a) shows the scatter plot that showcases the probability of a sample being classified as cancer using this unique tumor-associated dendritic cell signature. It can be observed from FIG. 14, a) that the classification is accurate. FIG. 14, b) shows the calibration curve of the machine learning model's performance for cancer diagnosis using the unique signature of Tumor-associated dendritic cells. Based on the machine learning model, 100% sensitivity and specificity were achieved. Next, the principal components contributing to accurate cancer diagnosis were analyzed. It can be observed from FIG. 14, c) that the primary principal component, PC3, shows the maximum contribution in assessing the probability of a sample being diagnosed with cancer. Further, to assess the machine learning model's performance, the ROC curve was analyzed. It can be observed from FIG. 14, d) that the area under the curve was 1.00, confirming the accurate cancer classification using tumor-associated dendritic cell signature.

Next, the unique tumor-associated dendritic cell signature was used to assess the applicability of the signature for early-stage cancer diagnosis, as initially indicated in FIG. 13, c). The phenotype of dendritic cells (DCs) in early-stage cancer has been studied. DCs are a diverse group of specialized antigen-presenting cells with key roles in the initiation and regulation of innate and adaptive immune responses^{25,27}. The phenotype of DCs in cancer is heterogeneous, and different subsets of DCs have been identified that drive specific types of immune responses^{28,30}

FIG. 14, e) shows the scatter plot that showcases the probability of a sample being classified as early-stage cancer using a tumor-associated dendritic cell signature. It can be observed that the tumor-associated dendritic cell signature provides an accurate classification of early-stage cancer and can distinguish between early-stage and noncancer samples with 97.1% sensitivity and 96.7% specificity (FIG. 14, f). In analyzing the principal components contributing to the accurate diagnosis of early-stage cancer, PC1 and PC6 were identified as the primary factors for early-stage cancer diagnosis (FIG. 14, g). Next, the model's accuracy using the ROC Curve was analyzed, where the area under the curve was of 0.998 (FIG. 14, h).

### Example 5—Tumor Associated B Cells for Cancer Diagnosis

B cells are a type of lymphocyte that produces antibodies to help the body fight infections and can be involved in the immune response to cancer³¹. The immune system can be divided into innate and adaptive immune systems, both contributing to the recognition and removal of foreign pathogens as well as tumors. Recent studies have shown that intratumoral B cells are present in human cancer. Studies have shown that B cells can present cognate tumor-derived antigens to T cells, with the functional consequences of such interactions being shaped by the B cell phenotype³². Hence, use of the SERS technique was employed to discover the unique signature of tumor-associated B cells and apply it to cancer diagnosis.

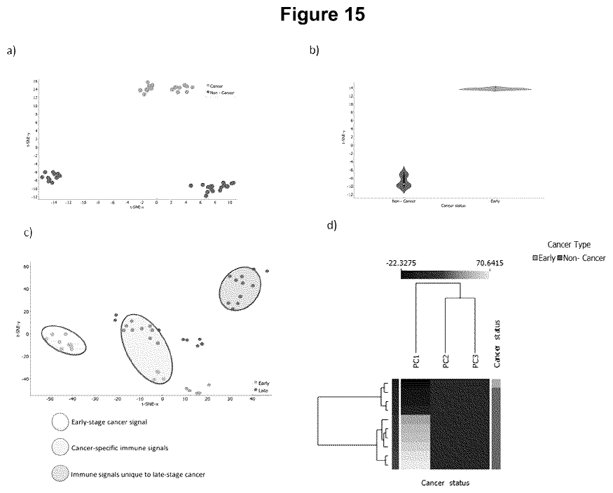

The tSNE analysis observed in FIG. 15, a) shows the variation in B cell phenotype between cancer and noncancer, showing that the Raman signature of tumor-associated B cells distinctly varies between noncancer and cancer (FIG. 15, b). By utilizing this distinct variation, tumor-associated B cells were used for comprehensive cancer assessment, including cancer diagnosis, detection of early-stage cancer and cancer tissue of origin, and for cancer metastasis detection. It can be observed in FIG. 15, c) that the signature of tumor-associated B cells also varies between early-stage and late-stage cancers. A phenotype specific to the cancer tissue of origin was also observed. Similarly, the heat map in FIG. 15, d) shows that most of the differences between cancer-associated B cells and non-cancer-associated B cells are contributed using PC1 corresponding to the peak positions presented in Table 4.

TABLE 4
The peak Raman shift positions of differences between cancer-
associated B cells and non-cancer-associated B cells.

| Raman Shift (1/cm) | Assignment |
|---|---|
| 677.94 | Ring breathing modes in the DNA bases |
| 850.41 | single bond stretching vibrations for the amino acids and valine |
| 1083.4 | C—N stretching mode of proteins |
| 1158 | C—C/C—N stretching (proteins) |
| 1420 | CH2 scissoring vibration (lipid band) |
| 1722.4 | C═O stretching (lipids) |

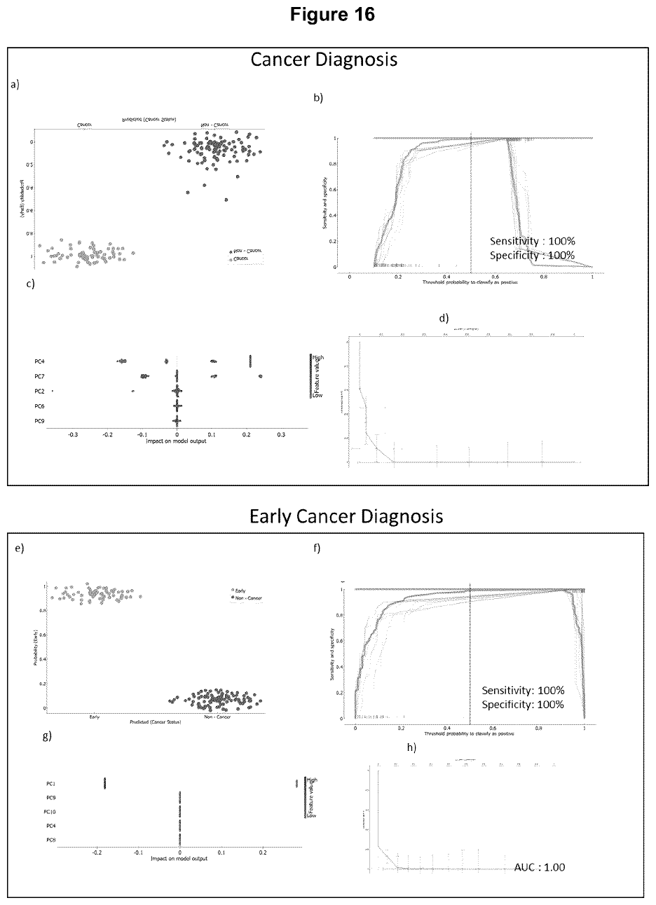

These unique differences between tumor-associated B cells and noncancer B cells were used for cancer diagnosis, as shown in FIG. 16. FIG. 16, a) shows the scatter plot that showcases the probability of a sample being classified as cancer using this unique tumor-associated B cell signature. It can be observed from FIG. 16, a) that the classification is accurate. FIG. 16, b) shows the calibration curve of the machine learning model's performance for cancer diagnosis using the unique signature of Tumor-associated B cell. Based on the machine learning model, 100% sensitivity and specificity were achieved (FIG. 16, b). Next, the principal components contributing to accurate cancer diagnosis were analyzed. It can be observed from FIG. 16, c) that the primary principal component, PC4, shows the maximum contribution in assessing the probability of a sample being diagnosed with cancer. Further, to assess the machine learning model's performance, the ROC curve was analyzed. It can be observed from FIG. 16, d) that the area under the curve was 1.00, confirming the accurate cancer classification using Tumor-associated B cell signature.

Next, the unique tumor-associated B cell signature was used to assess the applicability of the signature for early-stage cancer diagnosis, as initially indicated in FIG. 15, c). The phenotype of B cells in early-stage lung cancers, the varied landscape of B cells and plasma cells in the tumor appears to influence patient outcomes and treatment responses^{3,35}. Additionally, the presence of B cells in tertiary lymphoid structures (TLS) is associated with protective immunity in patients with lung cancer³⁶. In contrast, in some tumors, B cells in TLS form germinal centers and actively secrete antibodies that can recognize tumor-associated antigens, and patients whose tumor-associated TLS contain germinal centers sometimes have better prognoses³¹. The phenotype of B cells in different types of B-cell lymphoma can also vary depending on where the cancer cells are located. Overall, the phenotype of B cells in cancer could be influenced by the stage and type of cancer.

FIG. 16, e) shows the scatter plot that showcases the probability of a sample being classified as early-stage cancer using a tumor-associated B cell signature. It can be observed that the tumor-associated B cell signature provides an accurate classification of early-stage cancer and can distinguish between early-stage and noncancer samples with 100% sensitivity and specificity (FIG. 16, f). In analyzing the principal components contributing to the accurate diagnosis of early-stage cancer, PC1 was identified as the primary factor for early-stage cancer diagnosis (FIG. 16, g). Next, the model's accuracy was analyzed using the ROC Curve, where the area under the curve was of 1.00 (FIG. 16, h).

### Example 6—Tumor Associated Immune Cells for Multicancer Diagnosis and Identifying Cancer Tissue of Origin

Identifying the tissue of origin in cancer is important because it allows for more specific treatment and improves outcomes³⁷. Cancers of unknown primary (CUP) origin account for approximately 3% of all cancer diagnoses, and identifying the tissue of origin in these tumors is particularly challenging but crucial for effective treatment^{38,39}. Understanding the normal cellular hierarchy within a given tissue is an important prerequisite to identifying the cells of origin of cancers⁴⁰. While the combination of clinical data, tissue histology, and immunohistochemistry is usually sufficient for classifying cancers by tissue of origin, there remains a small but not insignificant proportion of difficult-to-classify cases⁴¹. Overall, identifying the tissue of origin in cancer is essential for determining the most appropriate treatment and improving patient outcomes.

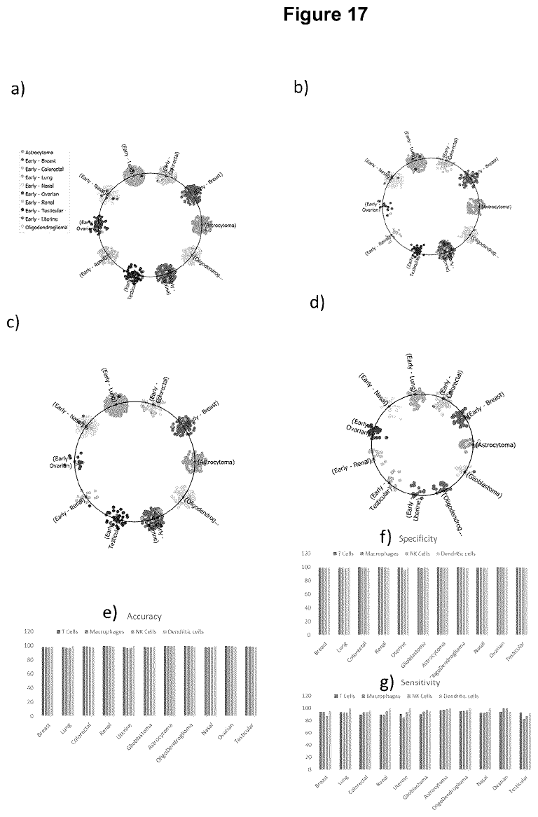

To identify the cancer tissue of origin using tumor associated immune cells, the machine learning algorithm was trained with the signatures of tumor associated immune cells including T cells (FIG. 17, a), macrophages (FIG. 17, b), dendritic cells (FIG. 17, c), NK cells (FIG. 14, d) and B cells (data not shown) to provide the probability of a sample to be from a specific cancer tissue of origin. These tumor associated immune cell signatures can distinguish cancer tissue of origin with more than 98% accuracy (FIG. 17, e) and more than 99% specificity (FIG. 17, f) and sensitivity (FIG. 17, g).

### Example 7—Diagnosis of Early-Stage Tumors Based on Size Using Tumor Associated Immune Cell Signature

One of the critical pitfalls of existing screening and early-stage diagnostic techniques is the lack of the ability to detect the tumors at early stages, when the size is a few mm or cm. When the tumor is smaller in size, the immune response will be in an active state, since the tumor defense mechanisms has not been activated. Hence, by utilizing signatures of the activated state of the immune response when the tumor size is small, this methodology could accurately detect cancer at a very early stage.

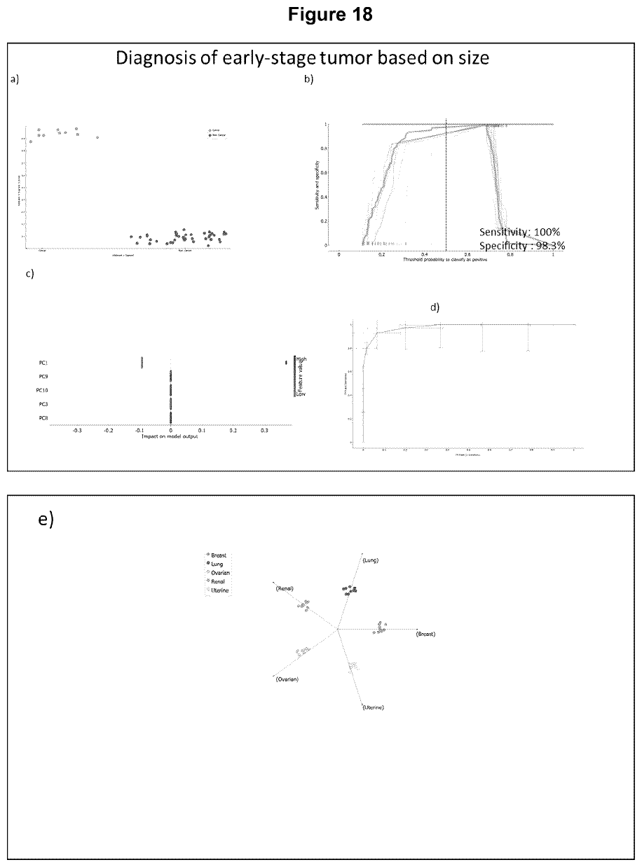

Indeed, FIG. 18 demonstrates the ability of tumor associated immune cell signatures to detect small sized tumors. It can be observed that the tumor-associated immune cell signature provides an accurate classification of early-stage cancer (FIG. 18, a) and can distinguish between early-stage and noncancer samples with 100% sensitivity and 98.3% specificity (FIG. 18, b). In analyzing the principal components contributing to the accurate diagnosis of early-stage cancer, PC1 was identified as the primary factor for early-stage cancer diagnosis (FIG. 18, c). Next, the model's accuracy was analyzed using the ROC Curve, where the area under the curve was of 1.00 (FIG. 18, d). The tumor tissue of origin for small tumors was classified with a 100% accuracy, specificity and sensitivity as observed in FIG. 18, e) and Table 5.

TABLE 5
Accuracy of multicancer diagnosis machine
learning model for small tumors.

| | | | | Tumor Size |
|---|---|---|---|---|
| Cancer Type | Specificity | Sensitivity | Accuracy | Range (cm) |
| Uterine | 100 | 100 | 100 | 0.5-2.4 |
| Breast | 100 | 100 | 100 | 2.4-5.0 |
| Lung | 100 | 100 | 100 | 2.4-6.3 |
| Ovarian | 100 | 100 | 100 | 0.5-1.6 |
| Renal | 100 | 100 | 100 | 1.5-2.2 |

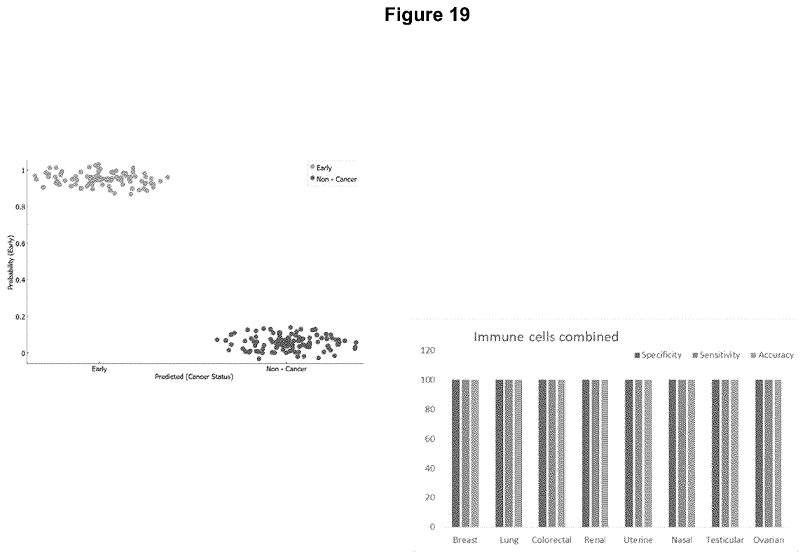

FIG. 19 demonstrates the applicability of tumor associated immune cell signature for multi-cancer diagnosis. Individually, tumor associated immune cells perform multi-cancer diagnosis with an accuracy of more than 95% (Table 6), specificity of more than 98% (Table 7), and sensitivity of more than 96% (Table 8). By combining the immune cell signatures of T cells, NK cells, B cells, dendritic cells, and macrophages, the performance of the tumor associated immune cell based multi-cancer diagnostic assay reached 100% (FIG. 19), eliminating false negatives.

TABLE 6
The accuracy of multicancer diagnosis
using immune cell signatures.

| Cancer Type | T Cells | Macrophages | NK Cells | Dendritic Cells |
|---|---|---|---|---|
| Breast | 98.9 | 100 | 99.7 | 100 |
| Lung | 100 | 99.7 | 98.3 | 99.7 |
| Colorectal | 97.3 | 98.8 | 95.7 | 98.9 |
| Renal | 100 | 100 | 100 | 100 |
| Uterine | 97.9 | 99.2 | 99.2 | 100 |
| Glioblastoma | 97.3 | 99.2 | 99.2 | 98.3 |
| Astrocytoma | 91.7 | 90.5 | 100 | 100 |
| Oligodendroglioma | 96.2 | 98.3 | 98.3 | 100 |
| Nasal | 100 | 98.9 | 98.9 | 100 |
| Ovarian | 99.3 | 100 | 100 | 97.8 |
| Testicular | 97.8 | 100 | 100 | 100 |

TABLE 7
The specificity of multicancer diagnosis
using immune cell signatures.

| Cancer Type | T Cells | Macrophages | NK Cells | Dendritic Cells |
|---|---|---|---|---|
| Breast | 98.9 | 100 | 99.7 | 100 |
| Lung | 100 | 99.7 | 98.3 | 99.7 |
| Colorectal | 97.3 | 98.8 | 95.7 | 95.8 |
| Renal | 100 | 100 | 100 | 100 |
| Uterine | 97.9 | 99.2 | 99.2 | 100 |
| Glioblastoma | 97.9 | 99.2 | 99.2 | 98.9 |
| Astrocytoma | 97.1 | 90.5 | 100 | 100 |
| Oligodendroglioma | 96.2 | 98.3 | 98.3 | 100 |
| Nasal | 100 | 98.9 | 98.9 | 100 |
| Ovarian | 99.3 | 100 | 100 | 98.5 |
| Testicular | 97.8 | 100 | 100 | 100 |

TABLE 8
The sensitivity of multicancer diagnosis
using immune cell signatures.

| Cancer Type | T Cells | Macrophages | NK Cells | Dendritic Cells |
|---|---|---|---|---|
| Breast | 94.4 | 100 | 99.7 | 100 |
| Lung | 100 | 99.7 | 98.3 | 100 |
| Colorectal | 100 | 98.8 | 95.7 | 100 |
| Renal | 100 | 100 | 100 | 100 |
| Uterine | 96.4 | 99.2 | 99.2 | 100 |
| Glioblastoma | 99.7 | 99.2 | 99.2 | 95.8 |
| Astrocytoma | 100 | 90.5 | 100 | 100 |
| Oligodendroglioma | 100 | 98.3 | 98.3 | 100 |
| Nasal | 100 | 98.9 | 98.9 | 100 |
| Ovarian | 98.6 | 100 | 100 | 95.5 |
| Testicular | 97.5 | 100 | 100 | 100 |

### Example 8—Cancer Diagnosis with Tumor Associated Neutrophils

### Materials and Methods

### Cell Culture

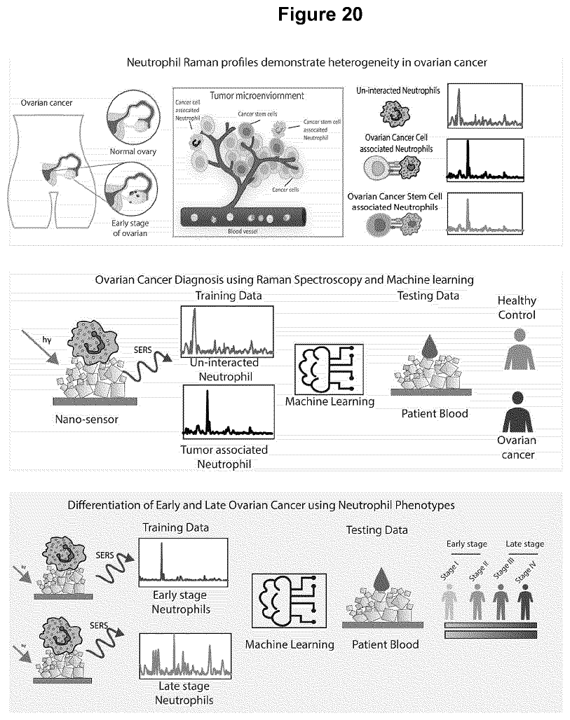

The neutrophil cells (Stem cell technologies) (HL60 cells) are a human cell line derived from peripheral blood lymphocytes. Cells were cultured in RPMI 1640+2 mM Glutamine+10-20% Foetal Bovine Serum (FBS). Cancer cell lines were obtained from ATCC for breast adenocarcinoma (MDA-MB231), non-small lung carcinoma (H69 AR) and colon adenocarcinoma (colo 205). Breast cancer cell line was maintained with DMEM with 10% FBS. The lung and colon cancer cell lines were maintained in RPMI 1640 with 10% FBS. All cells were cultured at 37 C in 5% C02 atmosphere. Raman spectral signatures were obtained from healthy neutrophils and neutrophils associated with pre-clinical models of cancer as demonstrated in the schematic of the experimental design in FIG. 20.

Inflammation is considered to be one of the hallmarks of cancer⁴². The complicated interaction of immune cells inside the tumor microenvironment enables ovarian cancer (OC) cells to rapidly colonize and propagate throughout the organ. Neutrophils are the classic inflammatory immune cells, accounting for 50-70 percent of circulating leucocytes⁴³. By interacting with the tumor microenvironment, neutrophils form a crucial link between inflammation and the development of cancer⁴⁴. Currently, it is believed that tumor-associated neutrophils (TANs) in the early stages of tumorigenesis exhibit anti-tumor properties, whereas TAN in the late stages of tumorigenesis, in which chronic inflammation has already developed, exhibit tumor promoting functions, such as proliferation, aggressiveness, and dissemination, as well as immune suppression⁴⁵. Similar to cancer cells that exhibit heterogeneity, it appears that the phenotype and functions of neutrophils fluctuate substantially with tumor growth, indicating the presence of neutrophil heterogeneity⁴⁴. In addition, neutrophils are short-lived cells with a lifespan of 24 hours, therefore, monitoring neutrophils can reveal the tumor's state in real time. Typically, the formation of pro- or anti-tumor neutrophils is driven by a change from a quiescent to an active cell state⁴⁶. It is therefore possible that the presence of tumor will be reflected in the biochemical and metabolic profile of neutrophils following activation. Indeed, by analyzing signals from the functional plasticity of TAN, it is possible to make an accurate cancer diagnosis, as demonstrated below.

This study shows that molecular profiles of Ovarian Tumor Associated Neutrophils (TAN) that have interacted with ovarian cancer and ovarian cancer stem cells provide diagnostic information for the detection of different stages of cancer. Neutrophils play a significant role in ovarian cancer immunosurveillance⁴⁷, and in response to signals from OC Tumor microenvironment, neutrophils cells are reprogrammed to either promote or suppress the tumor^{48,49}. This editing by tumor cells induces metabolic changes in the neutrophils cell resulting in altered Raman profiles. To detect these altered signals in the neutrophils, an ultrasensitive sensor capable of detecting low levels of TAN in circulation is needed. For this purpose, an in-house neutrophil profiler was developed as a scalable liquid biopsy platform. Raman spectroscopy (SERS) was used as the detection methodology (FIG. 20). SERS is a versatile spectroscopic technique characterized by non-destructive and rapid detection of analytes in circulation. Using SERS, two neutrophil Raman profiles were obtained, namely an un-interacted profile, and an ovarian cancer associated Raman profile for the detection of ovarian cancer. These phenotypic Raman subtypes of neutrophils form the basis for ovarian cancer detection using an ANN algorithm. Here, Neutrophils (HL-60) were co-cultured with Ovarian cancer (OVCAR) for 1, 6, 12 and 24 hours to obtain TAN profiles. Un-interacted HL-60 neutrophils were used as control to represent healthy neutrophils. Further, pro-tumor and anti-tumor Raman profiles of neutrophils were obtained by stimulating HL-60 with TGF-b and all-trans retinoic acid, respectively. These tumor profiles helped differentiate early and late-stage ovarian cancer. To validate the pro-tumor and anti-tumor profiles, the study was correlated using PDL-1 antibody. Thess validated and confirmed Raman profiles were then used as input training data for early and late stages of OC. To identify the different neutrophil signatures, a machine learning classifier was used to get tumor interacted signals from the Raman data of patient's blood. This was possible with the use of Artificial Neural Network (ANN) to classify the spectra into healthy and those associated with OC. The successful classification was obtained after training and the model provided sensitivity of 90% and specificity of 100% (Area Under the Curve (AUC)=1.00) with the test data.

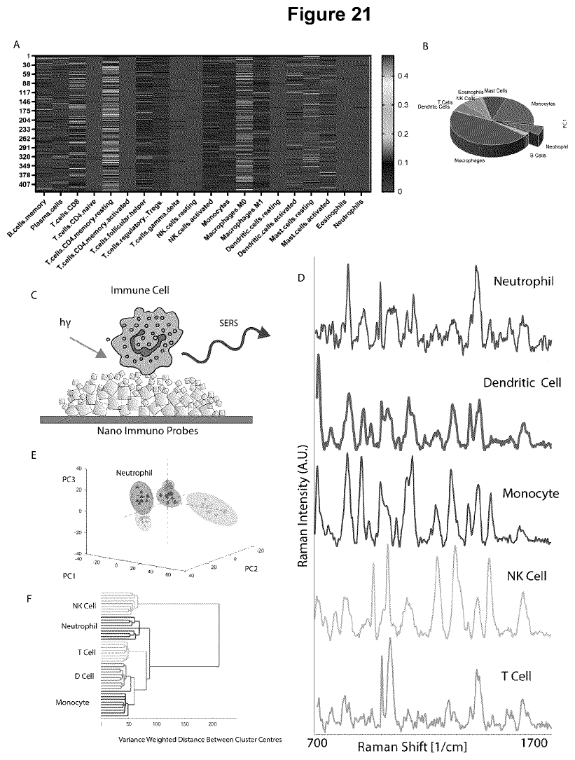

In the TME of OC a significant amount of immune cells infiltration can be seen^{50,51} The Cancer Genome Atlas (TCGA) of 430 Ovarian Cancer samples shows the presence of neutrophils in addition to other immune cells⁵². This data was obtained to through CIBERSORT analysis of 22 leucocyte subsets. FIG. 21, a) shows heatmap of immune cell infiltration found in ovarian cancer and FIG. 21, b) shows a pie chart with proportion of immune cells (neutrophils found in OC). These data collectively shows that neutrophils interact with ovarian tumor cells. Neutrophils, the most common leukocytes, are terminally differentiated effector cells⁵³. Neutrophils activate cytotoxic immunity⁵⁴ or NO production to halt primary tumor growth. As the tumor progresses, neutrophils signal through ROS, RNS, and proteases to promote tumorigenesis⁵⁵. Thus, neutrophils show metabolic adaptation as they move from circulation to intra-tumoral site⁵⁶. In order to obtain unique signatures of each individual immune cells, the Raman profile was taken using an in-house synthesized nano sensor (FIG. 21, c). From the peripheral blood, immune cells were isolated using commercially available kit and 5 μl of isolated immune cells dropped on the sensor and the Raman spectra were taken. FIG. 21, d) shows Raman spectra of neutrophil, dendritic cell, monocyte, natural killer cell and T cells. From the spectra changes in protein, lipids, and nucleic acids intensity were observed within the immune cells. The spectral differences between the Raman spectra of different leukocyte nuclei are subtle and imperceptible to the naked eye. Compared to lymphocytes (NK cells, T cells) that have a much larger nucleus that almost fills the cell, neutrophils have a small lobed nucleus⁵⁷⁻⁵⁹. Consequently, the vibrational bands of nucleic acid differ significantly between neutrophils and lymphocytes. In addition, since neutrophils are granulocytic cells protein contribution dominate neutrophil spectra while carotenoids bands predominate in lymphocyte spectra^{6,61}.

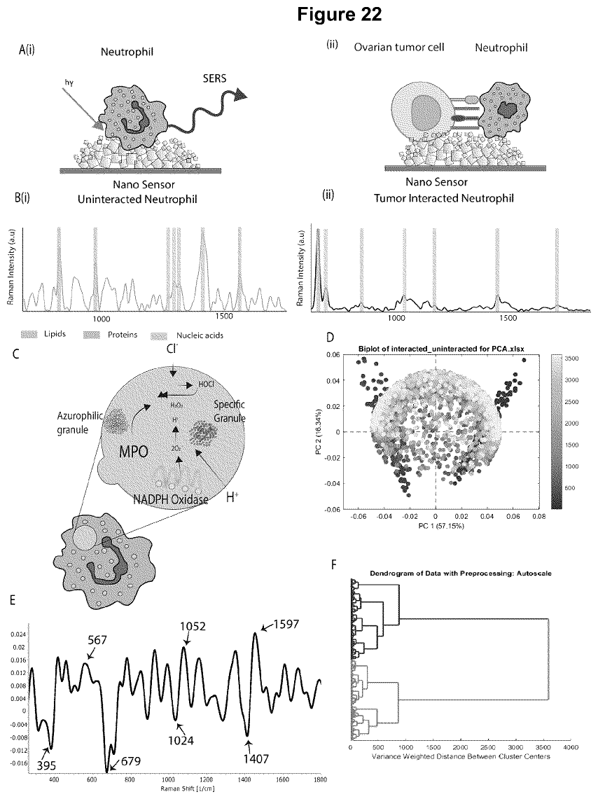

To obtain the metabolic phenotypes of tumor-associated neutrophils (TAN) HL60 (Human leukaemia cell line from ATCC) were differentiated into neutrophils. These neutrophils were co-cultured with OVCAR (ovarian cancer cell line from ATCC) to obtain ovarian tumor associated neutrophils. The un-interacted neutrophils are used as the control. Next, metabolic phenotypes existing in the control and TAN were analyzed using surface enhanced Raman spectroscopy (SERS). SERS is a non-destructive technique which is widely being used to analyze biological samples. SERS identifies the biochemical composition within these cells that can be used to identify the biochemical changes in different phenotypes of neutrophils in TME. FIG. 22, A(i) and A(ii) shows the schematics of un-interacted neutrophil and ovarian tumor associated neutrophils on the nano sensor, respectively. FIG. 22, B (i) and B (ii) shows the SERS spectra of un-interacted and ovarian tumor associated neutrophils. In stable conditions, neutrophils in circulation are not stimulated by outside stimuli. As a result, these cells are in a resting state, and the neutrophils used as controls in this experiment mimic these resting neutrophils. Several prominent peaks corresponding to protein, lipids and nucleic acid can be seen in the resting neutrophil spectra. Major contributors of resting Raman spectra come from cellular biomacromolecules like 672 cm⁻¹ corresponding to C—S stretching mode of cystine and cytosine, 1003 cm⁻¹ corresponding to phenylalanine and 2918 cm⁻¹ corresponding to asymmetric stretch of lipids and proteins.

Since SERS produces high dimensional data points, it is necessary to reduce the dimentionality of the data in order to extract the features prominent to TANs. For this purpose Principal Component Analysis (PCA) has proven to be a powerful dimensionality reduction technique suitable for SERS. FIG. 22, D is a biplot showing two distinct clusters for un-interacted neutrophils and TANs. From the loading plot of PCA (FIG. 22, E) it is apparent that TANs are characterized by a decrease in lipid contribution and an increase in nucleic acid contribution. Similar results were obtained using Hierarchical Cluster analysis (FIG. 22, F).

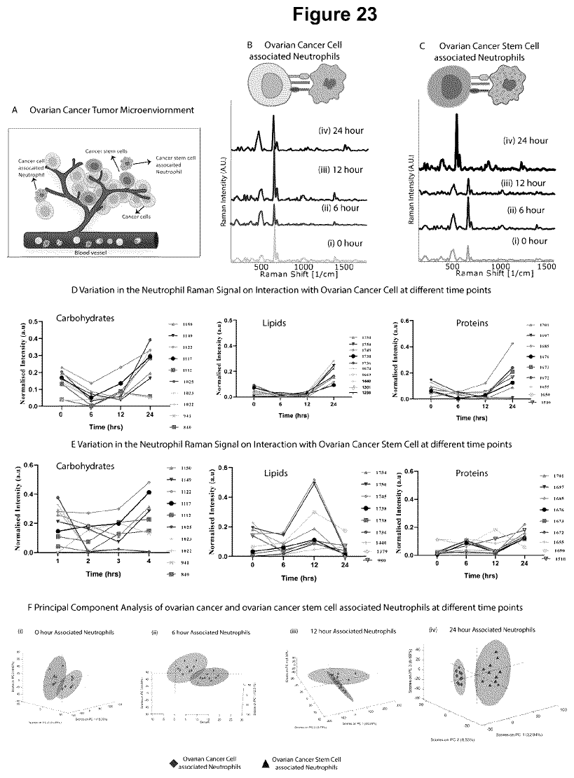

The ovarian cancer tumor microenvironment (TME) is a heterogenous population containing ovarian cancer cells, cancer stem cells, immune cells, endothelial cells, and fibroblasts to name a few (FIG. 23, A)⁶². When neutrophils infiltrate this ovarian cancer TME there is a shift in the phenotype depending on their interaction with cancer cell or cancer stem cells (FIG. 23, B and C, respectively). This shift in the metabolic phenotype occurs within a single day, resulting in a neutrophil population with distinct properties. In order to capture the shift in the metabolic phenotype, the Raman spectra of cancer interacted and cancer stem cell interacted neutrophils was taken at different time periods. Immediately upon interaction (0 Hr) there is a significant change in the Raman profile, implying that neutrophils undergo remarkable functional plasticity in a short period of time. Given the short life span of neutrophils (24 hrs), it is necessary for neutrophils to have dynamic participation in the TME in relatively short time.

The metabolic variations in cancer and CSCs were analyzed at different time points, including 0, 6, 12 and 24 hours. Neutrophils interacted with cancer cells have altered carbohydrates profile corresponding to 1122 cm, (C—O—C) polysaccharides and 1112 cm, saccharide band. These findings imply that there is significant glycolytic reprogramming occurring at different time points (FIG. 23, D). Similar reprogramming was also seen in neutrophils interacted with cancer stem cells (FIG. 23, E). These phenotypes can be attributed to the nutritional deprivations classically seen in the tumor microenvironment. Therefore, neutrophils have to compete for glucose energy source with cancer and cancer stem cells. Further, lipids and fatty acid metabolism was also analyzed in neutrophils interacting with the cancer and CSCs. Unlike glycolytic profile, lipid profile between cancer and CSC associated neutrophils show drastic variation between 0 and 24 hours (FIGS. 23, D and E, respectively). While TAN show gradual increase in lipids from 0 to 24 hours, CSC associated neutrophils show a decrease in lipid profile at 24 hours corresponding to 1745 cm, (phospholipids), 1750 cm, ((C═O), lipids in normal tissue). Next, metabolic changes associated with proteins were analyzed in neutrophils associated with cancer and CSCs (FIGS. 23, D and E, respectively). On observation it is apparent that there is a gradual increase in protein content from 0 to 24 hours in neutrophil associated with cancer, whereas in neutrophils associated with CSCs, two significant increases in protein content can be seen at 6 and 24 hours.

Since Raman spectroscopy generates multidimensional data, identification of features can be done effectively through PCA. FIG. 23, F shows variation in neutrophil Raman signals on interaction with ovarian cancer cell and CSC through PCA. Significant variation can be seen between the neutrophils associated with cancer and CSCs at 0, 6, 12 and, 24 hours. Clear clusters can be seen at all the time points observed showing that neutrophils acquire heterogenous phenotype depending on the cells of interaction and the duration of their interaction.

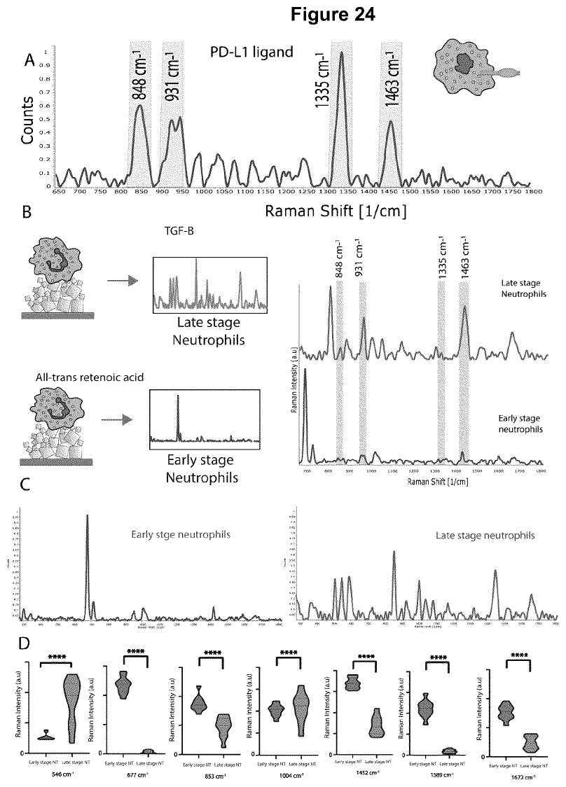

In this study, the immune check point protein programmed death ligand-1 (PD-L1) was used as an indicator for cancer interaction. PD-L1 is expressed in neutrophils upon tumour interactions⁶³ and hence the presence of signals of PD-L1 in neutrophils should be consistent with tumor interaction (FIG. 24, A). For this purpose, PD-L1 spectra were taken and the respective peaks were identified in early and late-stage neutrophils. From FIG. 24, A it is apparent the PDI-1 peaks are at 848 cm⁻¹, 931 cm⁻¹, 1335 cm⁻¹, and 1463 cm⁻¹. These PD-L1 peaks were recorded and identified in early and late-stage neutrophils. To stimulate early and late-stage neutrophils, in vitro neutrophils were cultured with all trans retinoic acid and TGFβ, respectively⁶⁴. SERS spectra were taken (FIG. 24, C) and PD-L1 peaks were identified in both early and late-stage neutrophils (FIG. 24, B). From the figure it is apparent that late-stage neutrophils show prominent PD-L1 peaks compared to early-stage neutrophils. Further univariate analysis was done using student t-test for early and late-stage neutrophils corresponding to 546 cm⁻¹, 677 cm⁻¹, 853 cm⁻¹, 1004 cm⁻¹, 1452 cm⁻¹, 1589 cm⁻¹, and 1672 cm⁻¹ (FIG. 24, D). All analyses show statistically significant differences (**** P<0.0001) between early and late-stage neutrophils.

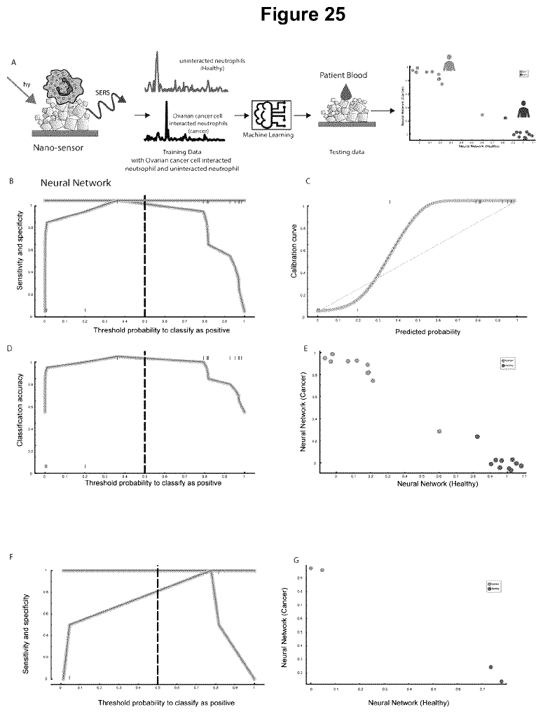

The ANN model diagnosed OC with 90% sensitivity and 100% specificity as shown in FIG. 25, B and Table 9. This deep learning based diagnostic model showed 95% classification accuracy (FIG. 25, D), Area under the curve was 1.000 and precision was 1.000. Clear clusters were observed between healthy, and cancer as shown in the ANN scatter plot (FIG. 25, E). The overlap of cancer subset in the healthy cluster was an indication of misclassification in one sample resulting 90% sensitivity (FIG. 25, B).

TABLE 9
Neural network model accuracy for OC detection.

| Model | AUC | CA | Sensitivity | Specificity | Precision | Recall |
|---|---|---|---|---|---|---|
| Neural | 1.000 | 0.950 | 0.900 | 1.000 | 1.000 | 0.900 |
| Network | | | | | | |

To further validate the above results, an ANN classification model was developed using blood from healthy and OC patients to classify OC and healthy for training and testing data sets. This yielded 100% sensitivity and 100% sensitivity with overall 100% classification accuracy (FIG. 24, F and Table 10). These results validate the ANN-based diagnostic model. The scatter plot (FIG. 25, G) shows distinct clusters with 100% precision.

TABLE 10
Neural network model accuracy using
patient data for OC detection.

| Model | AUC | CA | Sensitivity | Specificity | Precision | Recall |
|---|---|---|---|---|---|---|
| Neural | 1.000 | 1.000 | 1.000 | 1.000 | 1.000 | 1.000 |
| Network | | | | | | |

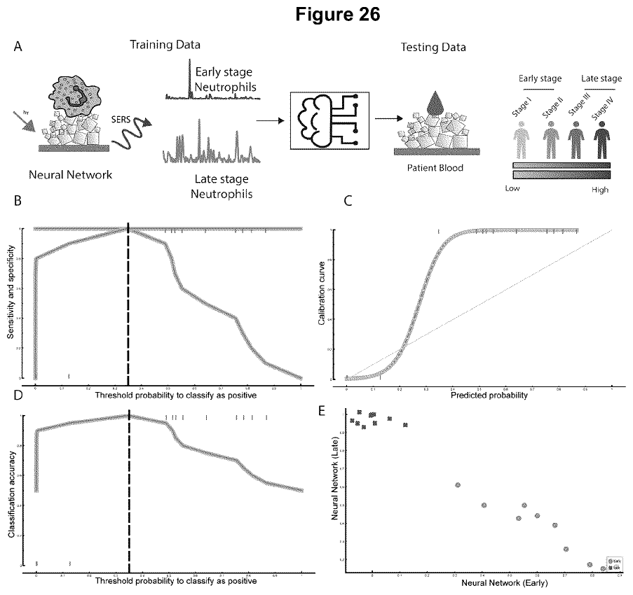

After successful prediction with OC diagnosis, the diagnostic model was tested for early OC detection. In order to classify early stage and late-stage OC, SERS data of early-stage neutrophils and late-stage neutrophils were used as training data. In the testing dataset, patient blood from early-stage OC and late-stage OC was used. The neural network scatter plot (FIG. 26, E) showed distinct clusters between early-stage OC and late-stage OC. The homogenous clusters (early and late) indicated high precision and accuracy during the classification. A total of 200 iterations with 100 neurons in hidden layers were performed and yielded 90% sensitivity and 100% specificity (FIG. 26, B). Overall classification accuracy peaks to 90% (FIG. 26, D) with Area Under the Curve (AUC) at 1.000. These results demonstrate that early-stage detection of OC is possible by using neutrophil as biomarker with machine learning based diagnostic model.

### Example 9—Immune Cell DNA as a Diagnostic Marker for Cancer Diagnosis

Immune cells have shown disease-selective activation through selective promoters primarily expressed in diseased cells (Ronald, J A et al., 2015). The disease selective activation causes changes to the expression profile of immune cell DNA, including changes to DNA methylation status and changes in the chemical structure. By detecting these changes to immune cell DNA in circulation, these inherent molecular changes may be useful as surrogate markers for cancer diagnosis.

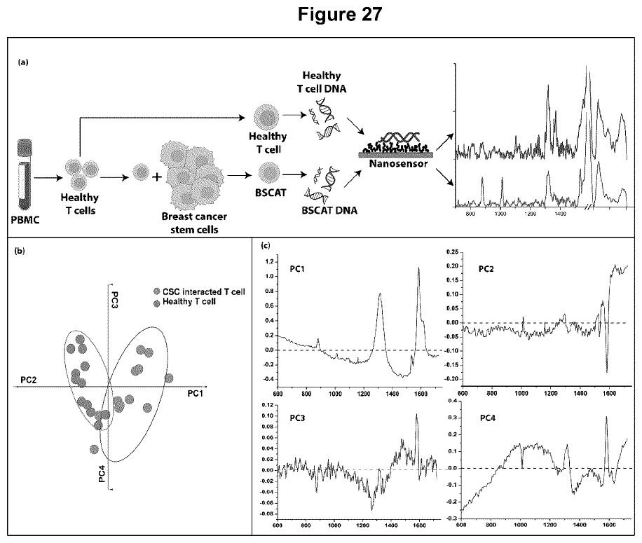

Studies have shown that T cells undergo drastic molecular level and surface receptor level changes after interaction with breast cancer stem cells. T cells were demonstrated to induce expression of stemness genes and genes associated with dedifferentitation.⁶⁵ T cells stimulates enhanced malignancy by bringing changes in the tumor at DNA and cellular levels leading to hyper progression of tumor. The collective molecular changes in the DNA of T cells interacted with breast cancer stem cells provides profound information regarding a disease state in an individual. Here, validation of T cell DNA as biomarker for breast cancer diagnosis was performed by isolating T cells from healthy PBMC (peripheral blood mononuclear cells) and coculturing it with breast cancer stem cells (BCSCs). The T cells after interaction with BCSC were isolated and the DNA from these T cells were isolated for further SERS analysis. SERS profiles of the DNA of healthy T cells and BCSC interacted T cells were acquired using the nano sensors to investigate the molecular level changes in the DNA of the T cells. The spectra of healthy T cells and T cells interacted with BCSC are shown in FIG. 27, a) which shows the differences in peak positions and intensities at 1098 cm⁻¹, 838 cm⁻¹ and 1276 cm⁻¹ corresponding to the DNA phosphorylation, adenine & thymine and cytosine peaks respectively, all of which are associated with the DNA changes.

Further, machine learning was applied to the SERS data of DNA to study the whole set of spectra so that differences between the healthy T cells and BCSC associated T cells could be established. Principal component analysis was performed, and the scatter plot is shown in FIG. 27, b) which shows that the spectra data points of healthy T cell DNA and BCSC associated T cell DNA were distinguished from each other except for a brief overlapping region. The minimal overlap is attributed to the common DNA features like DNA proteins, phosphates and the nitrogenous bases. The differences were observed as a whole spectrum which was contributed collectively by all the components in the genomic DNA. To further investigate the specific peak values contributing to the changes observed in the PCA analysis, the PC loading plots of the principal components PC1, PC2, PC3 and PC4 were analyzed. The PC loading plots are shown in FIG. 27, c). The peaks that were responsible for significant contribution to the changes as observed from the PC loading plots are 887, 1563, 1648, 1098, 1276 and 1376 cm⁻¹.^{66,67} The peak values observed in the loading plots corresponded to A&T, C, PO₂, G, Amino acid vibration and phosphodiester bridge, respectively.

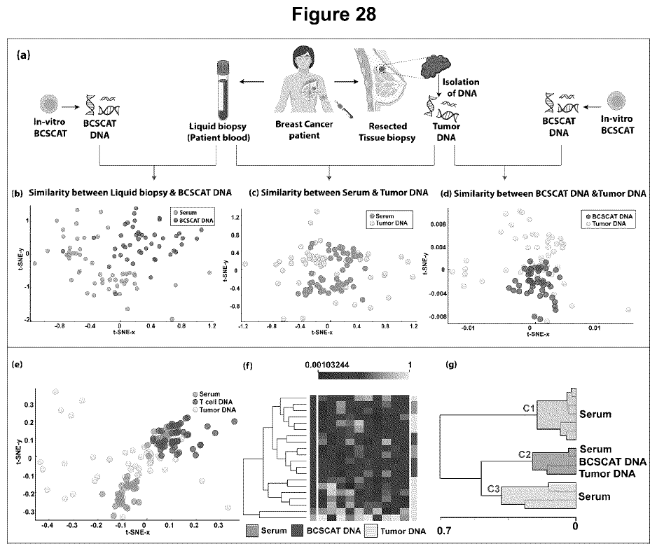

This study emphasized the use of liquid biopsy (peripheral blood) to detect tumor associated signals in circulation through cell free DNA present in plasma. To validate the feasibility of using liquid biopsy to reflect tumor signals, the similarities in signals existing between the patient tumor DNA, BCSCAT DNA and matched patient plasma (i.e., cell free DNA) were investigated. DNA was isolated from the patient tumor samples and from T cells interacted with breast cancer tumors and Raman spectra of the isolated DNA and of plasma were recorded. The data were analyzed to ascertain the similarity scores of the three types of samples. FIG. 27, a) shows an illustration of the study conducted to validate the biomarker in liquid biopsy. As seen throughout FIG. 28, b)-d), there is significant similarity between serum and BCSC associated T cell DNA (FIG. 28, b), serum and tumor DNA (FIG. 28, c), and BCSC associated T cell DNA and tumor DNA (FIG. 28, d), indicating that cell free DNA in plasma is a useful biomarker in cancer diagnosis.

Tumor microenvironment (TME) plays a pivotal role in tumor initiation, progress and metastasis. T cells form a substantial proportion of the breast TME performing contrast roles such as controlling tumor growth as well as promoting tumor growth by exerting immunosuppressive activities. The infiltrating T cells in breast cancer stem cells interact with the tumor cells and exhibit breast cancer associated features. Studies have established that there exists distinct difference in the phenotype and abundance of different types of T cells in different histological grades of breast cancer.⁶⁸⁻⁷⁰ They have shown that there is an increase in expression of FOXP3 and CD152 which are associated with regulatory T cell functions that are a cause for the immunosuppressive activity. Additionally, in higher grades, the increased levels of FOXP3+ Tregs are directly associated with poor prognosis and estrogen receptor negative status making it more challenging to diagnose and treat.⁷¹

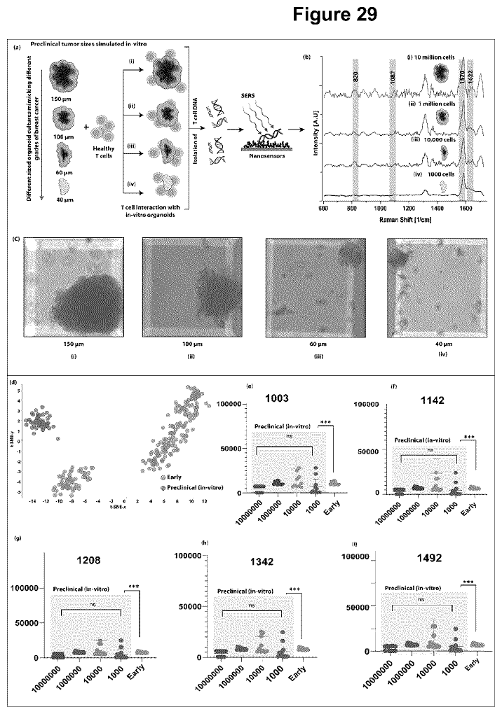

Since there is a significant and distinct change reported in the T cell phenotype and abundance in different grades, 4 sizes of tumor organoids were mimicked in vitro using the heterogenous population of primary breast cancer cell line BT 474, as shown in FIG. 29, a). The cancer stem cell enriched heterogenous cells were counted and seeded in aggrewell plates to form tumor organoids of known sizes. The seeding density of the cells were 10 million, 1 million, 10,000 and 1000 cells per well in aggrewell plates supplemented in serum free medium to promote the formation of tumor organoids. The organoid cultures were then treated with T cells isolated from healthy PBMC for 24 hours to enable T cell and breast cancer TME interaction. The T cells were then isolated, and DNA was isolated from the T cells interacted with different sized tumor cultures. SERS spectra of the DNA were recorded using the nanosensors and the peaks are shown in FIG. 29, b). The DNA spectral profile shows peaks at 820, 1087, 1318, 1570 and 1622 cm⁻¹ which correspond to citrate, PO2- symmetric stretching of the phosphodiester bonds, adenine ring stretching, adenine and guanine, and thymine and cytosine peaks, respectively.^{67,72,73} These peaks corresponding to the DNA peaks are observed in the T cells interacted with different sized breast cancer tumors as shown in FIG. 29, c).

The 4 significant peaks are shown individually in FIG. 29, b) to show the reduction in intensity of the peaks with reduction in size of the breast cancer tumor that the T cells have interacted with. The smallest tumor still showed a positive effect on the T cells which is observed in the form of the peak intensities and the DNA corresponding wavenumbers (FIG. 29, e)-i). In contrast, these peaks were not observed in the DNA of a healthy or an un interacted T cells which clearly establishes the genomic level change that T cells undergo upon interaction with the tumors irrespective of their size and number of cells in the TME. These subtle differences between the DNA of healthy and tumor interacted T cells can be utilized as a biomarker for breast cancer detection. Since the smallest tumor was able to exert a change in phenotype and genotype of T cells, this biomarker is a reliable and accurate one for early diagnosis and prognosis of breast cancer.

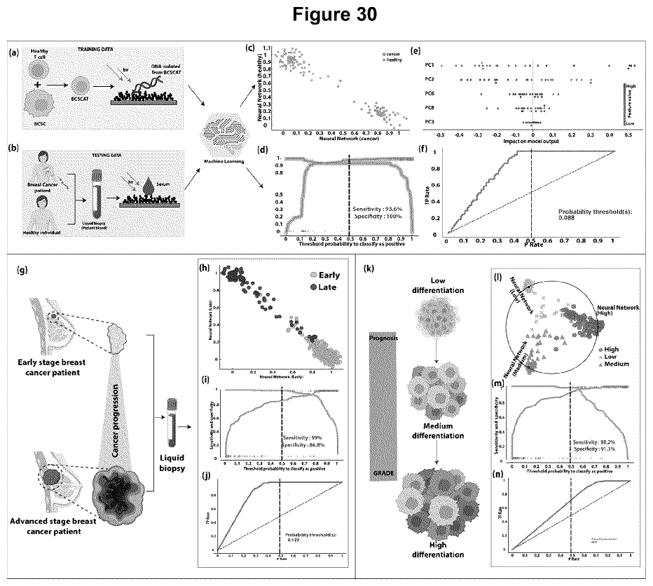

Next, the in vitro model generated for early diagnosis of breast cancer was applied and the ability to detect the differentiation levels of cancer cells in early stage tumors was assessed. It can be observed form FIG. 30 that the immune cell DNA based methodology for early diagnosis of breast cancer could be performed with 93.6% sensitivity and 100% specificity (FIG. 30, d). The cancer stage could be detected with a 99% sensitivity and 86.8% specificity (FIG. 30, i). Similarly, the cellular differentiation levels in early stage breast cancer could be detected with 98.2% sensitivity and 91.3% specificity (FIG. 30, m).

### Example 10—Immune Cell DNA for Detection of Cancer Metastasis

The tumor immune microenvironment is a crucial part of the development and metastasis of breast cancer. For example, studies have shown that tumor-infiltrating lymphocytes were associated with a better prognosis in breast cancer⁷⁴. In addition, the immune cells infiltrating the tumor are easily accessible markers for the prognosis of breast cancer^{75,76}. Hence, these immune cell signatures, especially the immunogenomic signatures, may provide a comprehensive and reliable way to determine the prognosis and metastasis of breast cancer.

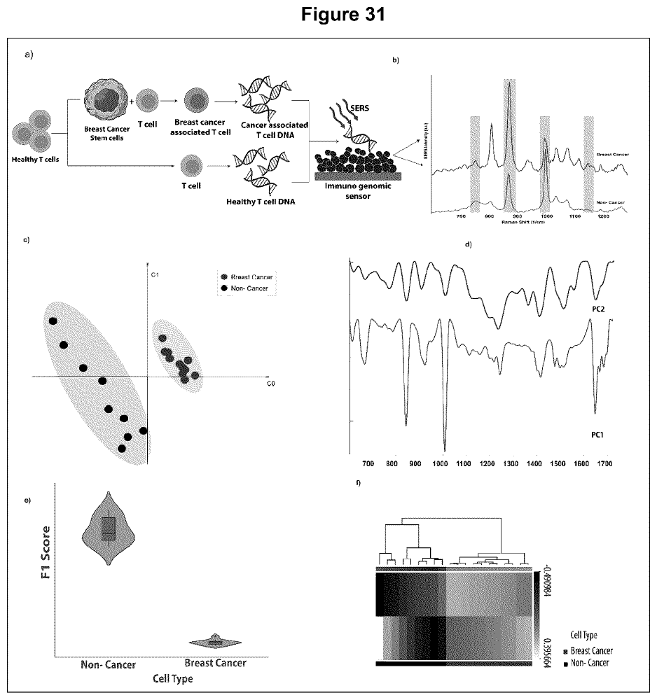

The immunogenomic signature unique to breast cancer was developed by coculturing naïve T cells with breast cancer stem cell-enriched spheroids generated from in vitro models (MDA-MB-231, MDA-MB 361). Breast cancer stem cell-enriched spheroids were adopted to account for the tumor heterogeneity observed in tumor samples. The DNA isolated from the T cells cocultured with breast cancer stem cell spheroids was used to obtain the immunogenomic SERS signature presented in FIG. 31, b). The representative SERS spectra presented in FIG. 31, b) shows the characteristic peaks associated with DNA. Further, compared with the DNA isolated from naïve T cells visual differences in the O—P—O bond at 831 cm⁻¹ and the cytosine residue at 1173 cm⁻¹ were observed, as reported in the literature⁷⁷. Clustering analysis was also performed by applying a robust local linear embedding algorithm due to the high dimensionality of the SERS data⁷⁸ and the ability of the algorithm to reduce the influence of noise on the clustering. It can be observed from FIG. 31, c) that the immunogenomic signature associated with breast cancer forms a distinct cluster without overlapping with the immunogenomic signature of naïve T cells. This observation shows that the genomic signature of immune cells changes when encountering tumor cells.

Further, the PC loading analysis pinpoints the specific SERS peaks accountable for the differences in the immunogenomic signature of breast cancer (FIG. 31, d). Additionally, a correlation analysis was performed to study the contribution of heterogeneity in the unique immunogenomic signature of breast cancer, which separates into two distinct clusters representing the presence of genotypic heterogeneity in both non-cancer and breast cancer-associated immunogenomic signatures (FIG. 31, f). The F1 score comparison shows that breast cancer has a statistically significantly (p<0.0001) low score confirming the differentiation between the immunogenomic signatures (FIG. 31, e).

Breast cancer is one of the most versatile cancers phenotypically, characterized by the different responses to therapeutics, pathological features, and survival⁷⁹. Histologically, breast cancer is characterized by the expression of surface receptors such as estrogen receptor (ER), progesterone receptor (PR), and human epidermal growth factor receptor 2 (HER2)^{80,81}. In particular, the prognosis and aggressiveness of breast cancer are highly associated with the HER2 expression⁸². In addition, HER2 expression is assessed routinely to decide on treatment options. Hence, whether the immunogenomic signature differs for breast cancer subtypes was investigated. For this purpose, the genomic signature of naïve T cells cocultured with breast cancer stem cell-enriched spheroids generated from in vitro models of HER2 negative breast cancer (MDA-MB-231) and HER2 positive breast cancer (MDA-MB 361) were analyzed (Error! Reference source not found.2, a).

The representative SERS signature of the breast cancer subtypes in Error!Reference source not found.2, b) show characteristic peaks associated with DNA molecule. Due to the high-dimensional nature of the SERS signature and the close relationship with the analytes, a multidimensional scaling algorithm was used to pinpoint the peak positions that could differentiate the breast cancer subtypes (Error! Reference source not found.2, e). Based on the analysis, the key peak positions contributing to the difference in SERS signatures at 786 cm⁻¹, 1208 cm⁻¹, 1282 cm⁻¹, and 1421 cm⁻¹ could be attributed to DNA bases, ring breathing mode of A/T DNA bases, cytosine, adenine/guanine bases, respectively⁷⁷. Furthermore, on comparing the normalized SERS intensity of the peak positions mentioned above (Error! Reference source not found.2, c), a statistically significant difference between HER2-positive breast cancer and HER2-negative breast cancer (p<0.0001) was observed.

Further, linear correlation analysis showed that the normalized intensity of peak positions 1208 cm⁻¹, 1282 cm⁻¹, and 1421 cm⁻¹ showed a positive correlation with the breast cancer subtypes. Conversely, the peak at 786 cm⁻¹ showed a negative correlation (Error! Reference source not found.2, d), consistent with the reported literature^{76,83,84}

HER2-positive breast cancer is among the most aggressive. It can potentially metastasize to multiple organs, including the brain, liver, and lymph nodes, at early stages with poor prognosis⁸⁷. Further, studies have pointed out that the leading cause of clinical inefficacy in treating HER2-positive breast cancer is spatial and temporal heterogeneity⁸⁸⁻⁹⁰. In particular, brain metastasis is incident in almost 50% of patients diagnosed with HER2-positive breast cancer^{91,92}, drastically reducing the overall survival of the patients. Furthermore, the prognosis of HER2-positive breast cancer is highly correlated with the immune landscape, especially the infiltration of immune cells 93. Hence, whether the immunogenomic signature is distinct for metastatic breast cancer was assessed.

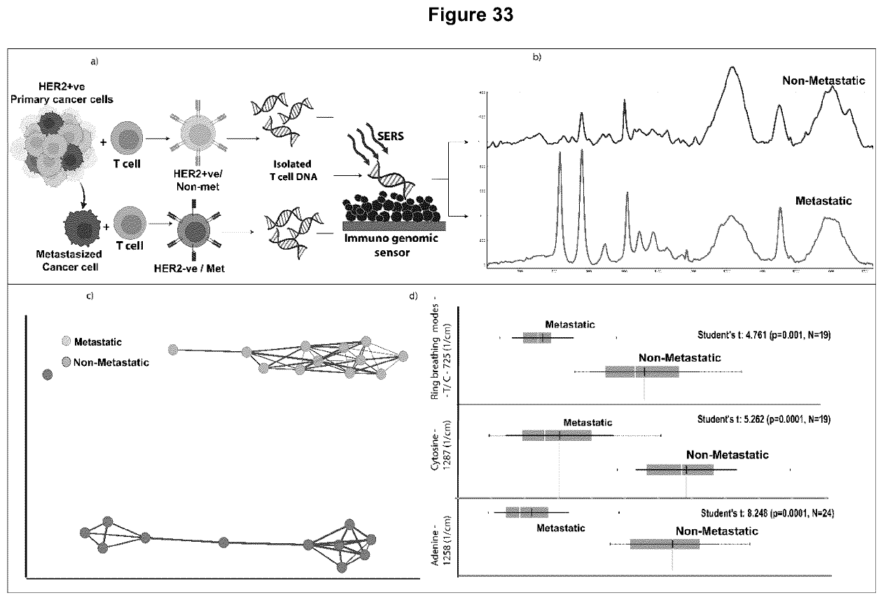

To obtain the immunogenomic signature specific to HER2-positive metastatic breast cancer, the genomic signature of naïve T cells cocultured with breast cancer stem cell-enriched spheroids generated from in vitro models of HER2-positive primary breast cancer (BT 474) and HER2-positive metastatic breast cancer (MDA-MB 361) was analyzed (FIG. 33, a). The SERS spectra in FIG. 33, b) show characteristic peaks associated with DNA consistent with those reported in literature^{77,94}. It can also be observed from FIG. 33, b) that the normalized intensity of metastatic breast cancer is significantly higher than that of primary breast cancer. In addition, a graph network analysis was performed to analyze the differences between primary and metastatic breast cancer. The network analysis presented in FIG. 33, c) shows that primary and metastatic breast cancer immunogenomic signatures differ significantly, without any overlap. Further, these analyses show overall connectivity to each of the samples from metastatic breast cancer. No interrelationships were observed outside the cluster.

Next, the highly connected nodes were selected to determine how the connectivity between primary and metastatic breast cancer is distinguished. Finally, the centrality indices were compared to inspect the statistical importance⁹⁵. The SERS peaks at 725 cm⁻¹, 1258 cm⁻¹, and 1287 cm⁻¹, corresponding to ring breathing modes of thymine/cytosine, Adenine, and cytosine, respectively^{77,96,97}. The comparison of normalized peak intensities revealed the significant difference between primary breast cancer and metastatic breast cancer (FIG. 33, d), which showed the ability of the immunogenomic signature to differentiate between HER2-positive primary and metastatic breast cancer. Research has emphasized the importance of HER2 status in metastasis and its effect on clinical prognosis^{98,99}. Combined with these results, the uniqueness of the immunogenomic signature of HER2-positive metastatic breast cancer can significantly open the possibility of serving as a predictive biomarker for managing HER2-positive metastatic breast cancer.

The immunogenomic signature obtained using in vitro breast cancer models provided preliminary data, which was then extended to clinical samples for applicability as a clinical diagnostic assay. Therefore, when assessing the translatability of metastatic breast cancer immunogenomic signature to clinical samples, the immunogenic signature obtained from in vitro breast cancer models was compared with clinical samples from patients diagnosed with metastatic breast cancer and primary breast cancer.

The hierarchical clustering algorithm was applied to evaluate the incidence of the metastatic immunogenomic signature in DNA isolated from tissue biopsy samples. Hierarchical clustering analysis is a robust algorithm that analyses multiple factors to classify samples based on similarities and differences and captures the tumor heterogeneity¹⁰⁰. The tissue biopsy DNA represents the tumor microenvironment and its heterogeneity. Primarily the immunogenomic signature is determined by the impact and interaction of immune cells in the tumor microenvironment¹⁰¹. In addition, the immune escape mechanism of breast cancer is regulated by the tumor microenvironment by forming an immunosuppressive state^{102,103}. Hence, to assess the validity of the immunogenomic signature of metastatic breast cancer, it is essential to establish similarity with the genomic signature of the tumor microenvironment.

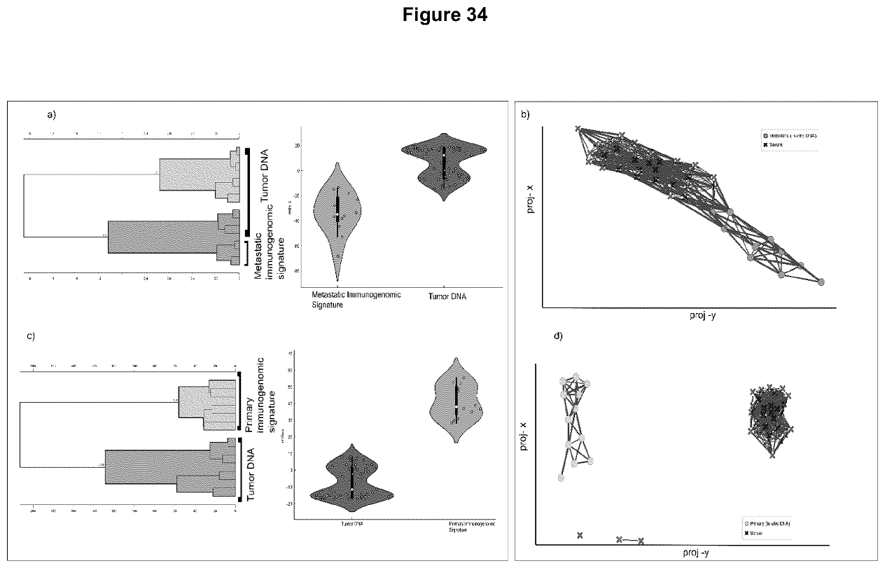

It can be noted from FIG. 34, a) that the metastatic immunogenomic signature shows high similarity with a subset of samples from the tissue biopsy, which could be attributed to the heterogeneity in the tissue biopsy samples. Subsequently, the immunogenomic signature of non-metastatic breast cancer was compared with the DNA from tissue biopsy samples. It can be observed from FIG. 34, c) that there exists a high degree of dissimilarity between the samples, resulting in the formation of two distinct clusters. Further, it can be established that the metastatic immunogenomic signature can be used as a valid marker for the detection and prognosis of metastatic breast cancer.

Similarly, the presence of metastatic immunogenomic signature in the serum was analyzed, which is essential for applying the metastatic immunogenomic signature for liquid biopsy. Multiple studies have noted the distinct immunophenotypes in metastatic breast cancer and, in some cases, its detrimental effect on the prognosis and patient survival^{76,79,81,99,104}. However, immunogenomic features' contribution to the circulating tumor DNA is still widely unexplored, hence so is their applicability as a liquid biopsy biomarker. Therefore, the similarity of metastatic immunogenomic signature with was compared with serum samples isolated from patients diagnosed with metastatic breast cancer (FIG. 34, b).

Due to the high dimensionality and complex nature of the data, a network analysis was employed to measure the similarity and validity of the metastatic immunogenomic signature. It can be observed from FIG. 34, b) that the metastatic immunogenomic signature and the signature of serum obtained from a patient diagnosed with metastatic breast cancer form a single network that is highly interconnected. In contrast, the immunogenomic signature of non-metastatic breast cancer formed a distinctly different network from the signature of serum obtained from a patient diagnosed with metastatic breast cancer (FIG. 34, d) with a high degree of disconnect. In addition, to assess the closeness between the samples, the centrality measurement was used, which was observed to be 0.9, confirming the similarity between the metastatic serum samples and the metastatic immunogenomic signature. In contrast, the centrality measure of 0 was observed between metastatic serum samples and the non-metastatic immunogenomic showing the distinctions between the sample sets. This indicates that immunogenomic alterations play a critical role in cancer progression and metastasis and can be assessed non-invasively.

In this study, by exploiting the circulating components of the immunogenomic signature associated with breast cancer metastasis, the goal is to extend the immunogenomic signature to predict the potential of primary breast cancer to metastasize at early stages, assess the phenotype of breast cancer and detect the site of metastasis. One critical factor influencing the metastatic potential of a primary tumor is the infiltration of immune cells. For example, T cells indirectly promote invasion and metastasis in breast cancer by activating epidermal growth factor signaling and affecting tumor-associated macrophages' phenotype¹⁰⁵. Similarly, immunosuppressive cells such as CD 4(+) CD25(+) Treg enables metastasis and are commonly chosen by tumors to inhibit immune destruction¹⁰⁶⁻¹⁰⁸. In addition, it is well known that to form distant metastases, the cancer cells intravasate, migrate to distant sites, and interact with immune cells in circulation¹⁰⁹. The cancer cells in circulation interact with CD8 T cells, suppressing their effector function¹¹⁰. The studies above show the immune system's critical role in forming distant metastasis. Although the role of the immune system is critical in the progression and metastasis of cancer, the composition of immune cells is relatively low in the tumor microenvironment and circulation. Hence, a deconvolution analysis was performed to isolate the immune cell composition in the genomic signature of the tissue biopsy DNA and the serum sample.

The tissue biopsy measured a genomic signature that reflected every cell in the sample. However, the heterogeneous nature of the bulk tissue complicates the interpretation and quantification of the contribution from immune cells in the tumor microenvironment¹¹¹. Consequently, understanding the composition of the cell types in the bulk tissue biopsy samples through deconvolution becomes critical¹¹².

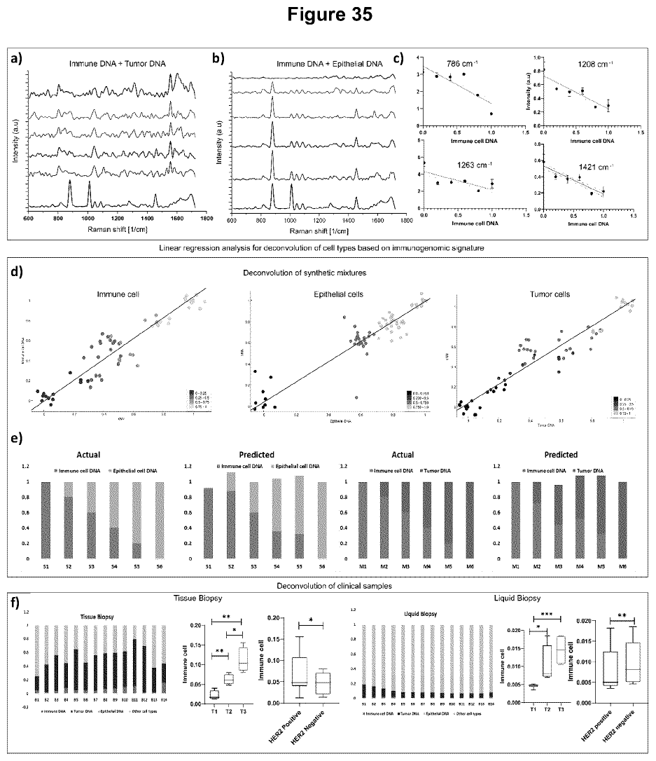

Using a quasi-supervised learning strategy, the training data was obtained for deconvolution using a synthetic mixture by controlling the percentage of tumor DNA, immune cell DNA, and epithelial cell DNA to account for the broader heterogeneity of the clinical samples. The representative spectra of the synthetic mixtures are presented in FIG. 35, a) and b), which show the characteristic peaks associated with DNA molecules. Further, the differences in the peak intensity can be visually observed when the composition varies. Subsequently, linear regression analyses were performed to determine the relationship between the varying immune cell DNA composition and SERS peak intensity (FIG. 35, c), which is critical to determine if the SERS spectral data can be used for deconvolution of cell types in a heterogeneous sample. The characteristic peaks associated with DNA molecules show a linear relationship with the varying immune cell DNA composition percentage. Hence, the linear regression algorithm was applied to assess the performance of the deconvolution algorithm using synthetic mixture data. In estimating the cell-type proportions, smaller differences were observed between the actual and predicted values for each type's percentages (FIG. 35, e). In addition, the RMSE was maintained at less than 10%, which validates the algorithm's superior performance.

Next, the algorithm was extended to clinical tissue and liquid biopsy samples to assess their applicability to complex samples. Finally, the synthetic mixture data was used to train the algorithm, which was validated using an independent cohort of clinical samples (FIG. 35, f). The results show a higher percentage of immune cell components in the tissue biopsy than in liquid biopsy samples, which could be attributed to the minimal presence of tumor-associated cell free DNA in circulation. Further, it demonstrates the ultra-high sensitivity of the nanosensors, which can detect the presence of even 0.005% of tumor-associated-immunogenomic signature in circulation.

On analyzing the tumor-associated immunogenomic signature in a tissue biopsy, the percentage of immune cells increases significantly with the tumor stage, with significant differences in the early stage (T1) metastatic samples. In addition, significant differences were observed in the immune cell levels between the HER2-positive and negative breast tumors (FIG. 35, f). This corroborates the uniqueness and the potential applicability of the metastatic immunogenomic signature for the diagnosis and clinical management of breast cancer, including assessing the metastatic potential, the phenotype of breast cancer, and the potential site of metastasis.

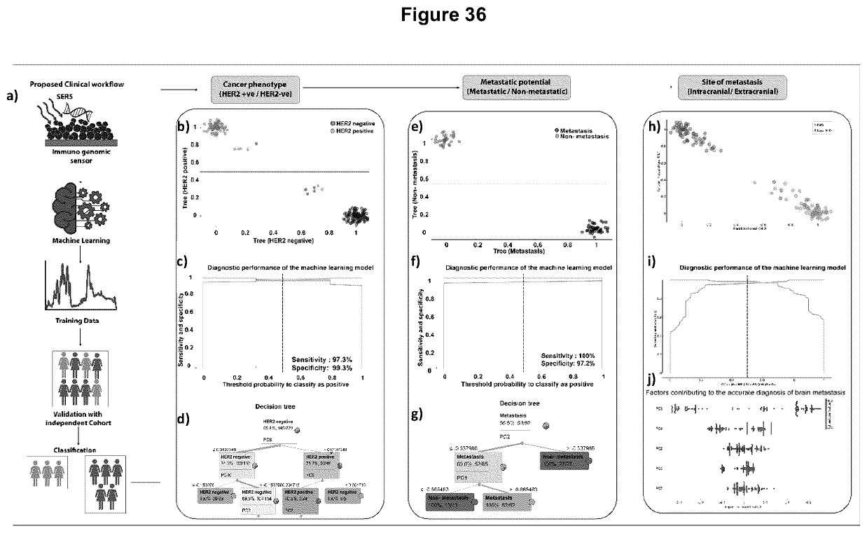

To assess the validity of the metastatic immunogenomic signature, a simple machine-learning algorithm was applied on an independent cohort of serum samples from patients diagnosed with metastatic breast cancer (n=20), non-metastatic breast cancer (n=20). The machine learning algorithm was trained using the metastatic immunogenomic signature generated using in vitro samples. It can be observed from FIG. 36 that the prediction of tumor metastasis from blood samples is achievable with high accuracy. Additionally, the specificity and sensitivity of the classification were at 97.2% and 100%, respectively (FIG. 36, middle panel).

The clinical impact of a false-positive result in liquid biopsy is enormous since it could lead to a misdiagnosis or affect the treatment regimen.¹³ Hence, reducing the false-positives is critical for an assay to be applied for the clinical management of cancer. Conventional liquid biopsy assay approaches suffer from a high false-positive rate, which is one of the major issues preventing the clinical realization of liquid biopsy. In this study, the false-positive rates were reduced by utilizing the metastatic immunogenomic signature to <0.1%. Next, the spectral features contributing to the high accuracy of predicting metastasis in breast cancers were investigated. The principal components (PC1 and PC2) provided the maximum variance between the sample groups resulting in an accurate prediction of cancer metastasis. The prevalent SERS peaks in PC1 and PC2 at 725 cm⁻¹, 1258 cm⁻¹, and 1287 cm⁻¹, corresponding to ring breathing modes of thymine/cytosine, Adenine, and cytosine, respectively^{77,96,97}.

Determining the phenotype of breast cancer at early stages is essential to determine the metastatic possibility and the probable site of metastasis. Studies have shown that the level of HER2 expression promotes an invasive phenotype and offers a selective advantage for metastasis during tumor initiation¹⁴. In this study, a simple machine-learning algorithm was applied that exhibited excellent performance, measured by the specificity and sensitivity between HER2 positive metastatic vs. HER2 negative metastatic breast cancer samples (FIG. 36). The peak positions prevalent in PC2 at peak positions 1208 cm⁻¹, 1282 cm⁻¹, and 1421 cm⁻¹ were the strongest predictor for the accurate Classification of breast cancer phenotype.

Metastatic HER2-positive breast cancer is one of the most aggressive forms of cancer⁹¹. In addition, patients with HER2-positive breast cancer show a high incidence of brain metastasis compared to patients with HER2-negative breast cancer. Further, screening for brain metastasis is currently not recommended due to a lack of data supporting the benefit in terms of overall survival¹¹⁵. Hence, a liquid biopsy test to assess the possibility of developing brain metastases could change the clinical management of brain metastasis. The high specificity of metastatic immunogenomic signature was used to validate if it can be applied to differentiate between intracranial metastatic disease and other tissue metastasis. By applying a simple machine-learning algorithm, the model could accurately differentiate the presence of brain metastasis, with a sensitivity and specificity of 98.5% and 96.7%, respectively (FIG. 36).

### Example 11—Detection of Cancer with Methylation Signals of Tumor Associated NK Cells

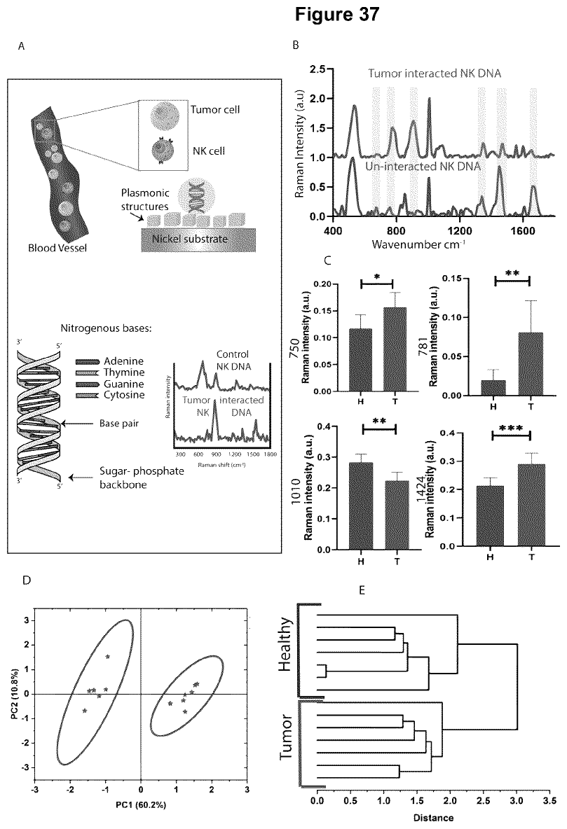

Using the in-house synthesized nano sensor, the Raman signature profiles of healthy immune cell (NK cell) DNA and colorectal tumor-interacted NK cell DNA were obtained by placing 5 μL of DNA sample onto the nano sensor and SERS spectral profile was acquired at 785 nm wavelength. (FIG. 37, A). Surface functionalization (—OH, C—OH, C—O, C═O) of the 3D nanoprobe facilitates improved interaction of the cell to the surface of the sensor. With the help of Raman spectroscopy, the nitrogenous base pairs adenine, guanine, thymine, and cytosine can be found alongside the phosphate backbone.^{27,28} In this study, the SERS spectra of the DNA isolated from in vitro Natural Killer cells (NK-92) that interacted with colorectal cancer cells (Colo-205) and healthy tumor un-interacted Natural Killer cells (Control NK-92) were captured in order to determine the genomic differences between them (FIG. 37, B). Characteristic nucleic acid Raman spectra bands with an A, T, C, G, and phosphate backbone were present in NK cells that are completely devoid of any association with tumor cells. Prominent peaks include 745 cm⁻¹ (adenine (A), thymine (T), and cytosine (C) ring breathing), 1485 cm⁻¹ and 1577 cm⁻¹ (A and guanine (G) contributions), and 785 cm⁻¹ 1090 cm⁻¹, and 1115 cm⁻¹ from the O P O backbone. The PO₂ peak at 1090 cm⁻¹ was determined to be the internal standard, which aids in removing variations from absolute intensity measurements. The PO₂ band is used as an internal benchmark since DNA structural damage has minimal effect on it. Using a two-tailed student t-test (FIG. 37, C), prominent spectral points revealed statistically significant differences between tumor-associated NK DNA and NK DNA (* 0.05, **0.01 and ***0.001 for 750 cm⁻¹, 781 cm⁻¹, 1010 cm⁻¹, and 1424 cm⁻¹ respectively). By subtracting this healthy NK DNA spectra points from the tumor-interacted NK cell DNA spectra, the contributions of each peak to the spectral difference was determined (FIG. 37). The spectral intensity of NK cell DNA associated with Colo rectal tumors was slightly different from that of NK cells not associated with tumors. As evidenced by the SERS profile, substantial changes occur at the genomic level in NK cell activity in the presence of Colorectal cancer cells²⁹. Spectral changes are caused by these intracellular changes driven by NK DNA.

NK cells are subject to epigenetic regulation in the tumor microenvironment. The epigenetic state of NK cells either helps in immune clearance or promotes cancer progression. Among the epigenetic modifications, DNA methylation is an essential epigenetic alteration that determines the cellular characteristics. DNA methylation is an indispensable epigenetic modification that determines cellular characteristics. DNA methylation signals are distinct due to their chemical stability and the insertion of methyl groups at the fifth position of cytosine and the sixth position of adenine. At the tumor microenvironment, NK cells develop a unique methylation profile that can be used to detect the presence of cancer.

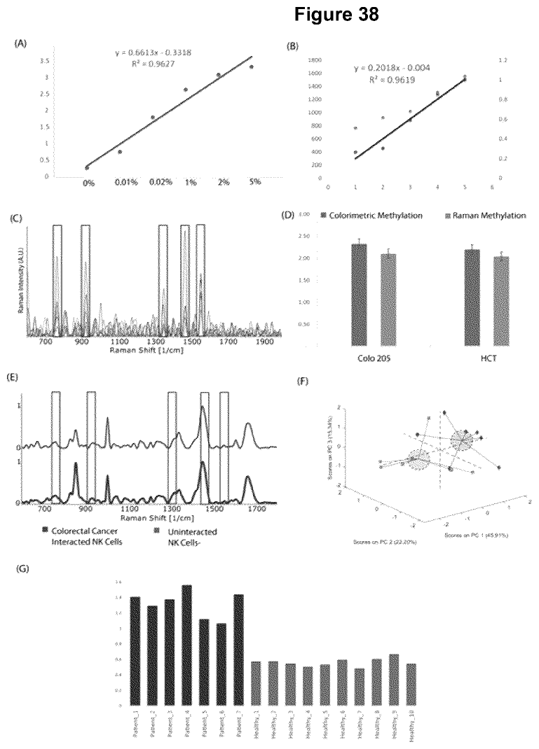

The capability of the nano sensor to detect molecular changes in DNA permits the further study the capability to estimate methylation changes in NK cells. Prior to measuring the DNA methylation status of NK cells, methylated and non-methylated DNA standards were analyzed using Raman spectroscopy. The optimal standard curve was generated using a 5-mC standard control based on the optical density at 450 nm (FIG. 38, A). Using Raman peak intensity and methylation percentages, a linear correlation model was then built. The Raman spectra of 5mC and methylated DNA (FIG. 38, C) revealed different levels of methylation with increased signals in the regions 700 cm⁻¹ to 1800 cm⁻¹. The methylation percentage in the DNA sample was calculated using the linear fit of the standard curve (R²=0.9619) and the equation y=0.2018x+0.004 (FIG. 38, B). As shown in FIG. 38, E, among the numerous DNA characteristics, 5 unique peaks were identified at 765 cm⁻¹, 924 cm⁻¹, 1020 cm⁻¹, 1310 cm⁻¹ and 1555 cm⁻¹, which correspond to the adenine ring and cytosine ring breathing modes. These peaks were considered as methylation specific markers as the variation in peak intensities and peak shift can be attributed to methylation-induced structural modification. The relative spectral intensities of methylation markers for colorectal tumor interacted NK cells were elevated in comparison to tumor un-interacted NK cell DNA. Multivariate analysis using PCA was done using the methylation specific Raman markers. i Two separate clusters were obtained showing healthy NK cell DNA and tumor interacted NK cell DNA (FIG. 38, F. The difference in peaks also implies that a methyl group was inserted at the position of the cytosine nucleotide. In order to validate the in vitro study, the methylation markers in patient blood samples from these experiments with patient DNA samples were examined. As anticipated, colorectal cancer patients exhibited elevated methylation of immune cells (FIG. 38, G).

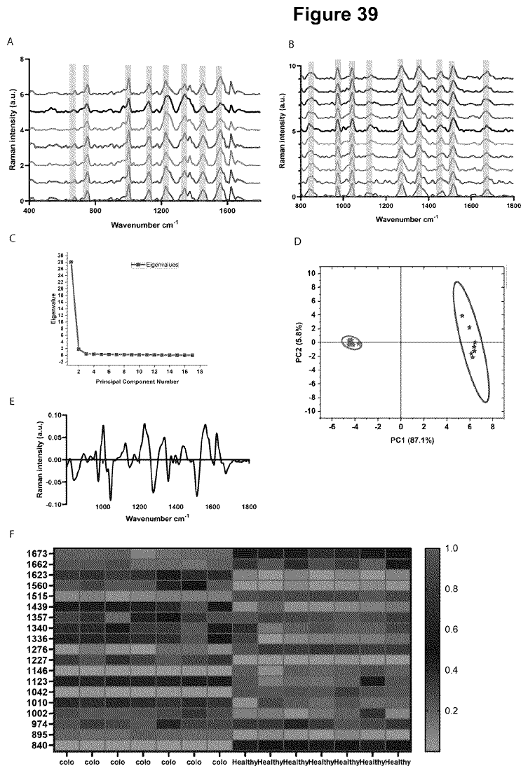

Given that the molecular changes in DNA and methylation-related changes in DNA of NK cells that have interacted with colon tumors (colo-205 co-cultured with NK-92) differ from those of NK cells that have not interacted with colon tumors (control NK-92), the next step was to test whether the identified markers could produce the same results in patient samples. SERS spectra were obtained from 20 clinical samples obtained from the Ontario Tumor Board (OTB) for this purpose. The DNA was extracted from the peripheral blood of cancer patients diagnosed by clinical presentation and histopathological diagnosis. The patient cohort had colorectal carcinoma (n=7) and healthy (n=10) volunteers. After dropping 5 μL of patient sample onto the sensor, Raman spectra were collected for each of the 17 samples. The Raman spectra for the patient samples are displayed in FIGS. 39, A and B, respectively, with genomic DNA molecular and methylation markers highlighted. In order to perform PCA, the collective Raman spectra of the molecular and methylation markers were used. The PCA results demonstrated clear clustering of the patient samples into the two distinct groups (FIG. 39, D). The peaks that were responsible for the differences were subsequently obtained from the loading plot plotting the coefficients of each variable in the first component against the coefficients in the second component (FIG. 39, E). The Raman spectra of colorectal cancer patients' samples and healthy volunteers' samples were compared using a heat map (FIG. 39, F). Each cell in the heat map is color-coded based on the spectral intensity values. The heat map clearly illustrates the distinctions between the two sample types.

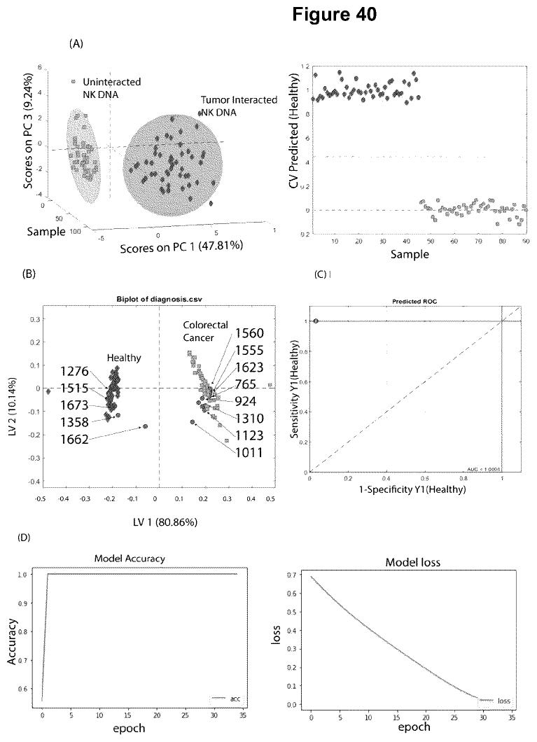

Using DNA structure and methylation peaks, an exploratory approach based on Principal Component Analysis was applied to the Raman spectra of invitro colorectal tumor interacted NK cell DNA (CNKDNA) and tumor un-interacted NK cell DNA (NKDNA). The first two Principal Components (PCs) were chosen, where two distinct clusters of CNKDNA (right) and NKDNA (left) can be seen (FIG. 40, A). It was possible to identify the variables responsible for sample clustering by examining the loadings of each variable on the first and second principal components, as shown in FIG. 40, B). This allowed for the identification of the variables responsible for sample clustering. The features that contributed the most variance were identified, and these features were then used to distinguish between cancer individuals and healthy individuals.

Following the completion of the initial stage of feature extraction with the help of PCA, PLS-DA models were developed in order to predict the class membership of newly collected observations. PLS-DA is a technique for supervised classification that looks for a direct correlation between the variable being studied and the response that is being predicted. The expected response for each of the two categories (n=2, healthy and cancer) is either 0 or 1. The samples were divided into calibration data sets and testing data sets, which allowed for the development of two different classification models. PLSDA was trained using the Raman peaks for DNA structure and methylation of patients' blood, utilizing venetian blind cross-validation. The training model was used to develop a classification system for cancer. Using the sample distribution of the calibration prediction generated by the PLS model, a threshold value of 0.5 was determined to be appropriate for classifying the data into their respective categories. Those samples that are above this threshold are classified as cancerous, whereas those that are below it are considered healthy. The results of the PLS-DA calibration model are displayed in FIG. 40, C). The analyses produced a prediction of 1.0, an accuracy of 100%, a precision of 100%, a specificity of 100%, and a sensitivity of 100%. The area under the curve was equal to 1, and the coefficient of determination (R²) for calibration was 0.99, while the coefficient of determination for prediction was 0.96. (FIG. 40, C).

In addition to PLSDA, Artificial Neural Networks (ANN) were used to show that the prediction is independent of the algorithm used. Because of their adaptability and capacity for independent learning, they have been utilized extensively in the identification and diagnosis of cancer. An artificial neural network is a multilayer network structure that is made up of many neurons that are connected to one another by a predetermined rule. Training was done with data from cell culture with cross-validation (80:20 splits). For predicting both healthy colorectal tissue and colorectal tumours, the model had a sensitivity and specificity of one hundred percent. Calculating the loss of the machine learning model requires using both the training and validation losses (FIG. 40, D). The precision of the model can be evaluated by contrasting the data that was predicted with the data that actually occurred. The loss of the model indicates how effectively or ineffectively it performs after each iteration, while the accuracy of the model serves as a measurement of how efficiently it performs.

### Example 12—Signature of GBM-Associated NK Cells for Cancer Diagnosis

### Materials and Methods

### Cell Lines and Co-Culture Assays

Nk-92 cells were obtained from ATCC (The American Tissue Type Culture Collection, USA). The source of the cells was from peripheral blood of non-Hodgkin's lymphoma. NK 92 cells resemble circulating NK cells in morphology and function. Alpha minimum essential medium w/o nucleosides with 0.2 mM myo-inositol, 0.1 mM 2-mercaptoethanol, 0.02 mM folic acid, 12.5 percent horse serum, and 12.5 percent foetal bovine serum were used to maintain the cells in culture. IL-2 was added at a concentration of 150 IU/ml to ensure adequate proliferation. A-172 glioblastoma cancer cell lines were obtained from ATCC and grown in DMEM media with 10% FBS. Both the cell lines were maintained in 5% CO₂ at 37° C. Transwell equipment (0.4 m hole size, corning, Lowell, MA) was used to coculture NK-92 cells with three breast cancer cell lines. The upper insert of a 24 well transwell plate was seeded with NK-92 cells.

### Isolation and Characterization of CIV

NK-92 cells were grown in prepared media for 24 hours. The cells were stimulated with IL-2 and total exosome isolation kit (Invitrogen) was used. Briefly, the supernatant was collected and incubated overnight with reagent from the kit at 2-8 C. The vesicles were then recovered from standard centrifugation at 10,000×g for 60 min. The pellet is then reconstituted in water for immediate further use. For TEM analysis, the CIV samples were adsorbed on a metal grid covered by lace like membrane and fixed with fixative to retain the shape and prevent osmotic damage. To improve the low contrast, negative staining is done with uranyl acetate contrast agent.

### SERS Data, Collection, Pre-Processing and Analysis

Renishaw invia confocal spectrometer with Leica DMI 6000 epifluorescence microscope was used to obtain SERS spectra. Spectrometer calibration was done using silicon chip with raman shift of 520 cm⁻¹. Crystal violet (CV) was prepared in different concentration and to collect the spectra of CV, NK cells and CIV, 5 ul of the respective samples were directly placed on the sensor. The spectra of cells and extracellular vesicles were taken in triplicates with 10 s acquisition time and averaged. 785 nm was used as excitation wavelength with spectral resolution of 1 cm⁻¹. Before analysis, the raw data was pre-processed SpectraGryph 1.2 for cosmic ray removal, smoothening (Savitsky-Golay) and baseline correction (adaptive).

### Statistical Analysis

Both multivariate and univariate analysis was performed wherever applicable. Multivariate statistics was performed using Solo eigenvector and univariate analysis was done using Prism Graphpad 9.0. All data are expressed as mean±standard deviation. For comparison of a single variable between two groups, students t test was used. Pearson test was used for correlation analysis. Multivariate analysis was done using principal component analysis (PCA). PCA displays the outliers (hotelling T2 test) and variance in the sample by taking all the features in the sample and component scores plot show the correlations within the sample. PLSDA was performed to distinguish categorial variables. Confidence ellipse with 95% confidence level was used for classification of the groups. From the samples 80% of original data was used as training data and 20% were used as model prediction purposes. The calibration model was cross validated for errors in calibration (training data) and error in cross validation (test data) The predictive performance of the model was checked with cross validation data. Root mean square of cross validation RMSECV showed the quality of the model. The performance of the model was confirmed with sensitivity, specificity, receiver operating characteristic curves (ROC), and area under the curve (AUC) for training, cross validation and test set. The variable's (VIP) Variable of Importance in PLSDA score was computed from the weighted sum of the squared correlations between the PLS-DA components and the original variable. The weights were obtained from the percentage of variation from the PLS-DA components. Artificial neural network (ANN) was applied to Raman peaks derived from PCA using python's keras, numpy and sklearn. Generative adversarial networks (GAN) were used for data augmentation.

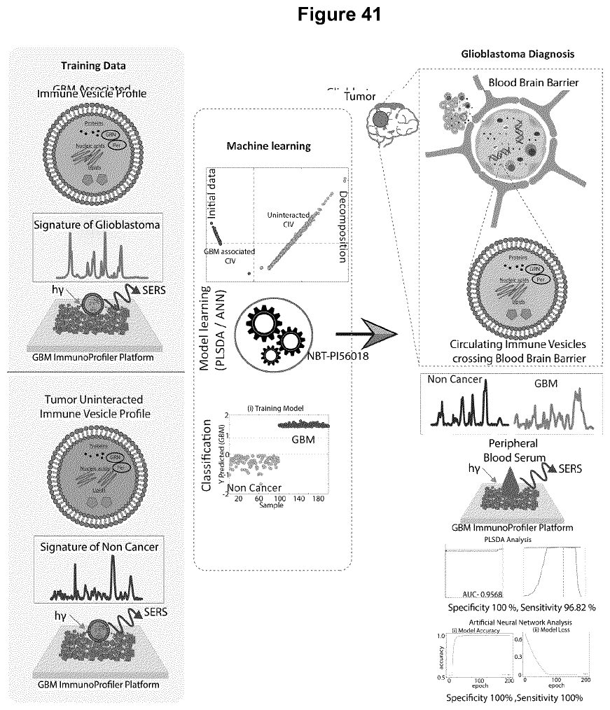

This Example shows that molecular profiles of circulating immune cell vesicles that have interacted with glioblastoma tumor microenvironment (TME) provide diagnostic information for the detection of glioblastoma. NK cells play an important role in glioblastoma immunosurveillance and respond to signals from GBM. NK cells are reprogrammed to either promote or suppress the tumor. This editing by tumor cells induces metabolic changes in the NK cell resulting in altered proteins, lipids, and nucleic acids within the cell. Further, these altered metabolites are selectively packaged into the vesicles of NK cells, giving rise to circulating immune vesicles (CIVs) with a unique metabolic profile. To detect CIVs, an ultrasensitive sensor capable of detecting low levels of analytes in circulation is needed. For this purpose, Surface Enhanced Raman Spectroscopy (SERS) was used as the detection method. SERS is a versatile spectroscopic technique characterized by non-destructive and rapid detection of analytes in circulation. As per FIG. 41, using SERS, two phenotypic spectra were obtained—naïve CIV spectra and GBM associated CIV spectra. These phenotypic subtypes of CIV form the basis for GBM detection in this Example. This diagnostic technique is illustrated in the graphical abstract, FIG. 41. To identify the different CIV signatures, a machine learning classifier was used to get tumor associated signals from the Raman data. This was possible with the use of partial least squares discriminative analysis (PLS-DA) to classify the spectra into those that are healthy and those associated with GBM. NK-92 cells were cocultured with A172 glioblastoma cells to obtain GBM-CIV, and their Raman data was used as input training data while the healthy CIVs were used as control. Successful classification was obtained after training, and the model provided 100% sensitivity and 100% specificity (AUC=1.0) with the test data.

### Characterization of NK Cell Derived Immune Vesicles

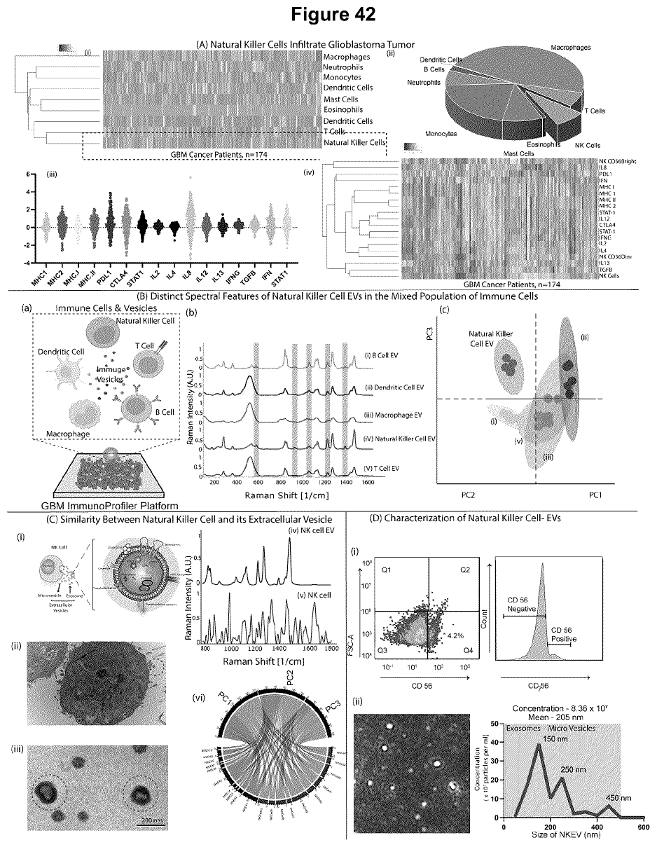

CIV from parent NK-92 cells that were stimulated with interleukin-2 (IL-2) were generated, as activated NK cells are known to produce more quantities of EVs (Pirzadeh Z, et al., 2014). As per FIG. 42, D), isolated EVs were characterized by Nanoparticle Tracking Analysis (NTA) and electron microscopy. NTA measured the size of larger vesicles (microvesicles) and smaller vesicles (exosomes) through a digital camera that records the Brownian motion of individual particles and calculates the diameter of the particles using the Stokes-Einstein equation (Himstedt R, et al., 2017). The size of NK-92 cell-derived microvesicles ranged from 150 nm to 450 nm, and the size of NK-92 cell-derived exosomes ranged from 50 to 100 nm, similar to the size of microvesicles and exosomes described elsewhere (He S, et al., 2019; Pirzadeh Z, et al., 2014). A snapshot of the NTA video is also shown in FIG. 42, D). The vesicles appeared as electron-dense dark outer membrane and electro-lucent bright inner mass. Larger vesicles appeared to have cauliflower morphology due to the aggregation of proteins around the EVs (Shibue T and Weinberg R A, 2017). Collectively, the results from NTA and TEM show that the isolated vesicles in this Example had sizes ranging from 50 nm to 450 nm.

Biochemical Similarity of Immune Cell Vesicles with Parent NK Cells with SERS Profiling

NK cell (NKC) spectrum was compared with CIV spectrum to determine if CIV exhibit similar properties to the parent NK cell for diagnostic purposes (FIG. 42). The SERS spectra of NKC and CIV were analyzed for their biochemical composition. FIG. 42, C shows spectra of NKC and CIV, respectively, and their tentative assignments are shown in Table 11.

TABLE 11
SERS assignment for NK cells (NKC) and NK cell
derived extracellular vesicles (NKEVs).

| Raman shift (cm⁻¹) | | |
|---|---|---|
| NKC | NKEV | Raman Assignment |
| 761 | 761 | Tryptophan d (ring) |
| | | Ring breathing tryptophan (proteins) |
| 832 | 832 | Asymmetric O—P—O stretching, tyrosine |
| 893 | 893 | Backbone, C—C skeletal, Phosphodiester, |
| | | Deoxyribose |
| 1015 | 1015 | Stretching C—O ribose |
| 1141 | 1141 | Palmitic acid |
| | | Fatty acid |
| 1255 | 1255 | Lipids |
| 1359 | 1359 | Tryptophan |
| 1475 | 1477 | Lipids |
| 1582 | 1582 | d(C══C), phenylalanine |
| 1695 | 1703 | Amide I (turns and bands) |
| 2879 | 2879 | CH 2 asymmetric stretch of lipids and proteins |
| 2933 | 2933 | CH 2 asymmetric stretch |

NK cell spectra clearly show information on proteins, lipids, carbohydrates, and nucleic acids. Characteristic vibrational bands for proteins such as amide III (1224 cm⁻¹), amide I (1632 cm⁻¹), tryptophan (1359 cm⁻¹), tyrosine (859 cm⁻¹) are visible. Few nucleic acid contributions are seen at 678 cm⁻¹ corresponding to ring breathing modes in the DNA bases, 789 cm⁻¹ corresponding O—P—O stretching in DNA. An analysis of the spectral profile of CIV indicates that they have a biochemically similar composition to parent cells, as seen in FIG. 42, c) vi). The spectral composition of CIV is predominantly due to the rich phospholipid bilayer with cholesterol-containing nucleic acids and protein cargo. Accordingly, the spectra show vibrational bands at 1300 cm⁻¹ (δ CH2 twisting, wagging of phospholipid) and 719 cm⁻¹, corresponding to symmetric stretch vibration of choline group N+ (CH3)₃, characteristic for phospholipids. Nucleic acid peak at 669 cm⁻¹ corresponds to thymine, guanine contributions in DNA/RNA, O—P—O stretch DNA phosphodiester, 720 cm⁻¹ (DNA), and protein peaks at 644 cm⁻¹ corresponding to C—C twisting mode of tyrosine, 761 cm⁻¹ rings breathing tryptophan, 951 cm⁻¹ (a-helix of proteins), 1224 cm⁻¹ (amide Ill protein) and lipids with the band at 701 cm⁻¹ (cholesterol), 1255 cm⁻¹ (lipid) and 1300 cm⁻¹ corresponding to CH₂ twisting and wagging (lipids). To reduce the dimensionality of the data and allow easier interpretation, principal component analysis (PCA) is done. PCA compresses high dimensional data from SERS into a 3D plot by selecting the maximum variation within the data set and plotting them along the component direction. Although, PCA is done to show the maximum variance, here PCA was used to show the similarities between the spectra. The first three components (PC1, PC2, and PC3) were chosen to include a minimum of 70% of the variance. In the PCA score plot with 3 components, NKC and CIV samples were closely clustered, confirming the near-identical composition of the cell and vesicles. Additionally, to find the key similarities between the spectra, a chord diagram was plotted, enabling visualization of the similarities in the biochemical composition of the cell and EV spectra. FIG. 42, c) vi) shows the link between CIV and parent NK cell with selected principal components. the lower half of the circle represents the immune cell and its component. Connections shown in blue are biochemical similarities of CIV with the first three principal components, connections represented in red (NK cells) show a similar connection with the first three principal components, and the metabolites fall into different clusters, indicating changed metabolic profiles.

Significant Variation in SERS Profiles of Immune Vesicles Derived from GBM Associated NK Cells Compared to Immune Vesicles Derived from Un-Interacted NK Cells

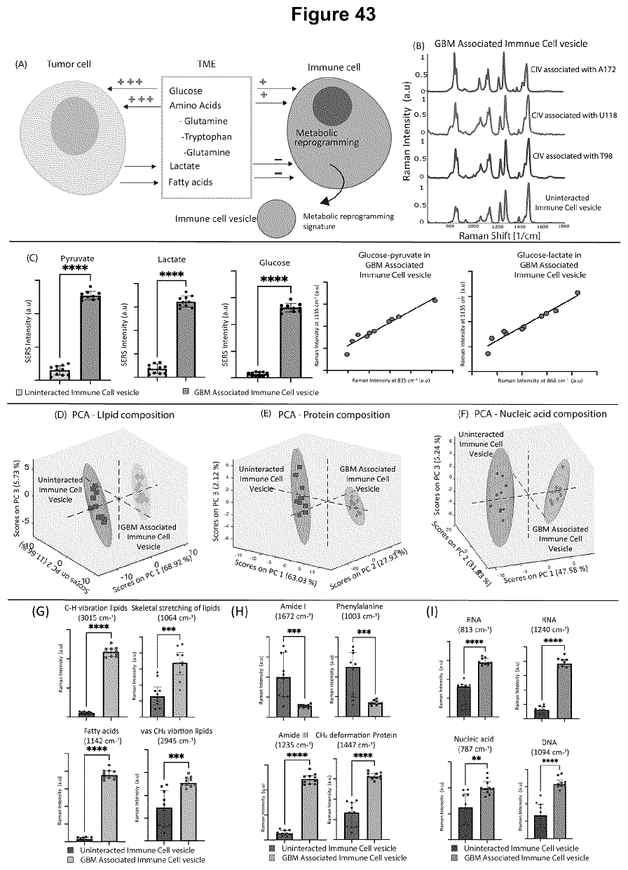

To understand how NK cells behave in the tumor microenvironment, it is important to know how NK cells identify, differentiate, and kill cancer cells. Consequently, their cellular metabolic expression varies by the type of cells they interact with. Example 2 demonstrated that NK cells exhibit different SERS profiles upon interaction with tumors cells due to differential education by cancer and undifferentiated cancer stem cells. Both resting and activated NK cells release extracellular vesicles (EVs) that contain parent metabolites. Metabolites inside CIVs are mainly proteins, lipids, and nucleic acids, sugars, and vitamins, and represent the metabolic state of the parent cell. The metabolic state of parent NK cells significantly affects the contents of the EVs that they generate. This is evident from the unique SERS spectra obtained from GBM-CIV (FIG. 43). Complete spectral assignment can be found in Table 12. To obtain the signature spectrum of Glioblastoma-associated NK cell-derived extracellular vesicles, NK-92 cells were exposed to A-172 Glioblastoma cells for 24 hours. Post interaction, the cell supernatant was collected, and centrifugation was done to obtain EVs and named as GBM-CIV. Un-interacted NK-92 cell supernatant was used as a control to obtain EVs and further named as CIV.

TABLE 12
SERS assignments for GBM-CIV.

| Peak (cm⁻¹) | Tentative SERS assignment |
|---|---|
| 535 | Cholesterol ester |
| 787 | RNA |
| 813 | C′5—O—P—O—C′3 phosphodiester bands in RNA |
| 844 | Tyrosine (Fermi resonance of ring fundamental and overtone) |
| 932 | Skeletal C—C, a -helix of proteins |
| 1003 | Phenylalanine, C—C skeletal |
| 1064 | Skeletal C—C stretch of lipids |
| 1094 | DNA |
| 1142 | v(C—N), proteins (protein assignment) |
| 1240 | RNA |
| 1235 | Amide III |
| 1281 | Amide III & CH2 wagging vibrations from glycine backbone & proline side chains |
| 1397 | C══O symmetric stretch |
| 1447 | CH2 deformation (protein vibration)-A marker for protein concentration |
| 1482 | Guanine and adenine ring breathing modes in the DNA bases |
| 1672 | Amide I band (C══O stretch coupled to a N—H bending) |
| 2887 | vs CH 3, lipids, fatty acids |
| 2945 | C—H vibrations in lipids & proteins |
| 3015 | CH of lipids |

In a nutrient-depleted environment, NK cells compete with GBM tumor cells for glucose and amino acids exhibiting the ‘Warburg phenomenon’ and metabolic switch to aerobic glycolysis (Ames E, et al., 2015; Tallerico R, et al., 2013; Butler K T, et al., 2018). Lactate and pyruvate, which are metabolites indicative of glycolysis, rise in NK cells after metabolic reprogramming. In line with this, as the cells transform into effector cells, the metabolic profile becomes increasingly critical. The SERS signature at 866 cm⁻¹ and 835 cm⁻¹, which correspond to lactate and pyruvate respectively, can be used to detect glycolytic reprogramming that gives rise to distinct SERS metabolic signatures. In GBM CIVs, lactate and pyruvate metabolite concentrations were considerably lower than in healthy CIVs (p<0.0001).

In addition, the relative change in these metabolites relative to glucose was accessed using intensity at 1125 cm⁻¹ (glucose) compared to the intensity at 866 cm⁻¹ (lactate) and 835 cm⁻¹ (pyruvate). The regression plots show a strong positive correlation (R²=0.9) between pyruvate-glucose and lactate-glucose (FIG. 43, C). Overall, the findings imply that glycolytic reprogramming distinguishes GBM CIVs from healthy CIVs, which sustains the increased energy demand caused by nutritional deprivation at the tumor site.

Fatty acid metabolism also plays a role in the development of different CIV phenotypes. Lipids form a major component of CIVs. However, in the TME, GBM cells release enormous amounts of fatty acids into the environment, which interfere with the activity of NK cells. Intracellular lipid accumulation lowers NK cells' cytotoxic function (Guo S, et al., 2021). In addition, changes in lipid content affect signal transduction pathways, membrane fluidity, and trafficking (Meza Ramirez CA, 2021). The changed immune response is due to a change in function, which is associated with changes in phenotype. The lipid contribution in GBM CIVs and healthy CIVs was investigated using SERS. Vibrations were at 3015 cm⁻¹, 2945 cm⁻¹, 1142 cm⁻¹ and 1064 cm⁻¹ corresponding to CH band, asymmetric vibration of CH₂, fatty acids and skeletal C—C stretch of lipids (acyl chains) respectively (FIG. 43, G).

When NK cells are exposed to tumor cells for a prolonged period, their immune responses are altered. Activation and inhibition of NK cells are brought about by a complex metabolism that necessitates a high degree of protein regulation. Several protein receptors and molecules (natural cytotoxicity receptors—NCR, cytotoxic proteins, CD16, CD56, NKp44, NKG2D, PDL1) are transferred to the extracellular vesicles in the form of lytic granules. These proteins can be detected on the surface or cytosol of CIVs. The secondary structure of proteins and the relative amounts of the different aromatic amino acids present were probed using SERS. The SERS peak at 1447 cm⁻¹ is an indication of protein concentration, and the most prominent changes were observed with amide I at 1672 cm⁻¹, amide Ill at 1235 cm⁻¹, and phenylalanine at 1003 cm⁻¹ (FIG. 43, H). While amide I and phenylalanine are significantly decreased in GBM-CIVs, the amide Ill peak is seen to increase in GBM-CIVs. Additionally, The entire SERS spectra were analyzed for visualization of the complete metabolic profile in the form of PCA. Lipids, proteins, and nucleic acids were explored using PCA score plots. PC1, PC2, and PC3 explained the maximum variance in the FIG. 43, D-F. The data within the same cluster were grouped close to each other, showing highly reproducible results. FIG. 43 shows clear differences in the lipid and protein compositions in the EV packaging. The results of univariate (two-tailed student t-test) and multivariate analyses (PCA) are shown in the FIG. 43, which demonstrate the distinct features. Collectively, the results demonstrate that metabolic signature from GBM-CIV can be used as a biomarker for GBM diagnosis.

### Validation of GBM Interaction

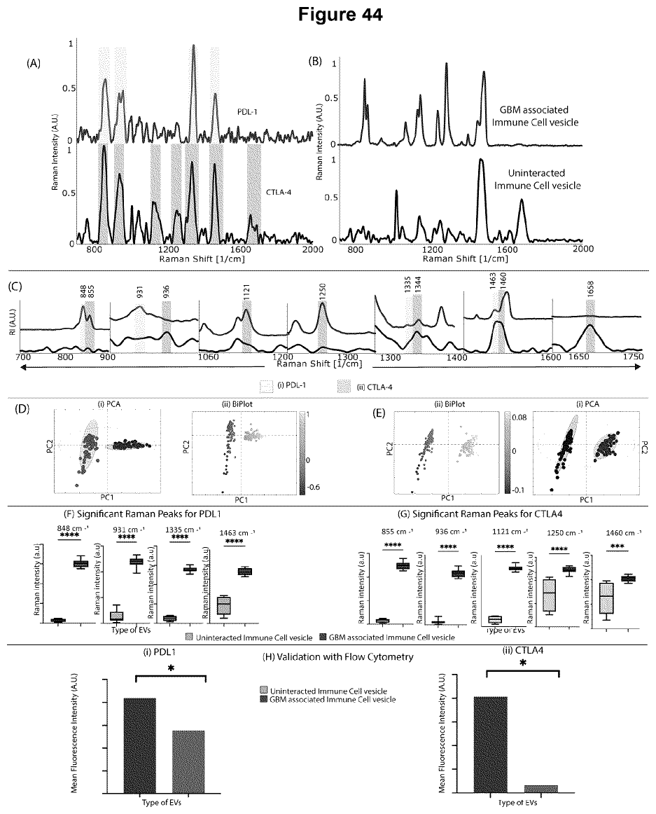

Classically, programmed death-ligand (PDL-1) has been found in tumors that have interacted with infiltrating immune cells as a way of evading detection. Because NK cells only express PDL-1 when they interact with tumors, PDL-1 expression in NK cells may be employed as a reliable indicator of cancer engagement. PDL-1 is also secreted in vesicles after being endocytosed. As a result, PDL-1 expression in GBM-CIV can be used to confirm the results of SERS. Expression of PDL-1 in GBM-CIVs was compared using SERS spectra from GBM-CIV and healthy CIV. FIG. 44, A) shows the Raman signature spectra of PDL-1. Prominent peaks appear at 846 cm-1 (tyrosine fermi resonance), 855 cm-1 (tyrosine v(C—C)), 952 cm⁻¹, 1083 cm⁻¹ (C—N stretching mode of proteins), 1125 cm⁻¹ (C—C stretching mode of proteins) and 1464 cm⁻¹ (Fermi interaction (bending CH₂ and stretching CH₂). The relative expression of PDL-1 in GBM associated CIV and healthy CIV was strongly significant (FIG. 44, F; two-tailed t-test with p<0.0001 and R²=0.99) and further supported by PCA and BiPlot (FIG. 44, D).

GBM Diagnosis with GBM Associated Circulating Immune Vesicles

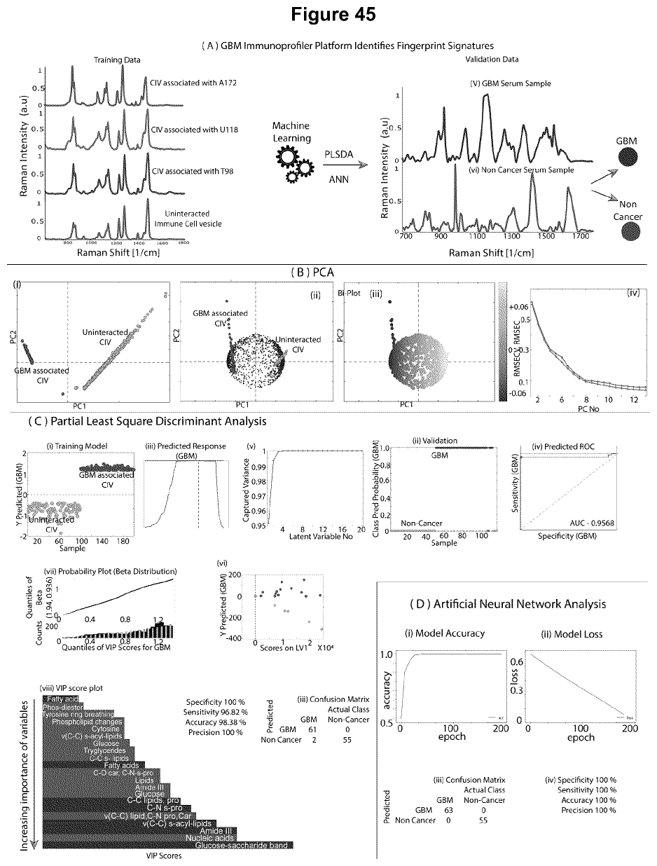

The classification models for GBM diagnosis were developed by applying supervised Partial Least Squares Discriminative Analysis (PLS-DA) to measure variation between the two phenotypes of CIV. PLS-DA was chosen since it is a powerful tool for chemometrics when the number of wavelengths exceeds the number of samples. PLS-DA finds mathematical models by combining partial least squares regression analysis and classification by discriminative analysis to assign unknown samples into different classes (0=Healthy and 1=GBM). FIG. 45, (A) shows the flowchart of PLS-DA describing the process of PLS regression model combined with discriminant analysis for prediction of glioblastoma. Two classes of spectral data were used as input training data: (1) GBM-CIV (cancer) and (2) CIV (healthy). Like the unsupervised data cluster, supervised PLS-DA showed similar clustering. The PLS-DA score plots differentiated CIV phenotypes with no overlap (FIG. 45, (B)(ii)). To look at the variables that cause maximum separation, the VIP scores were calculated. Variable of Importance in Projection (VIP) scores measure the importance of each variable in a PLS model's projection to discover key variables. In this model, a variable with VIP Scores of more than 1.4 was considered important. Several amino acids, lipids, and saccharides were predicted by the model as the variable of importance as in FIG. 45, (B)(iii). This shows that metabolites involved in NK cell metabolism may have a significant role in determining the diagnostic profile.

To evaluate the performance of the classification model and to obtain the total number of latent variables (LV), Venetian cross blinds were applied as the cross-validation (CV) method. Further, the original data was split into two independent datasets. 20% of original data were categorized as validation data and the remaining (80% data) were grouped as calibration data and used for model building.

Subsequently, the model was applied to the validation data and tested. Serum samples from GBM positive and healthy patients were used as external validation as input test data. Table 13 shows the output of the model in the form of a confusion matrix (Matthew's Correlation Coefficient=1.000). 0.42 was the threshold selected to classify the samples as GBM or healthy to evaluate the performance of the prediction model using the ROC curve AUC (AUC 0.9). Statistical results obtained from PLS-DA models are shown in Table 14 showing high R² values (0.99) and low error rate (RMSEP 0.02). Table 15 shows the classification accuracies of models. Accuracy was determined by the ratio of successful prediction to the total number of samples. The optimized PLS-DA model correctly predicted 10 GBM and 10 healthy samples among 20 samples. The 100% sensitivity and 100% specificity obtained from ROC for GBM detection are promising for future testing. Instead of relying on a single biomarker, metabolic signatures provide collective changes occurring in the disease thereby eliminating the possibility of random changes in a single metabolite to be correlated with disease. This diagnostic model has considered the collective spectral data, thereby eliminating the need to correlate a single biomarker for cancer detection.

TABLE 13
Model confusion matrix for GBM diagnosis.

| Class | TPR | FPR | TNR | FNR | Err |
|---|---|---|---|---|---|
| GBM | 1.00000 | 0.00000 | 1.00000 | 0.00000 | 0.00000 |
| Healthy | 1.00000 | 0.00000 | 1.00000 | 0.00000 | 0.00000 |

Actual Class

| Confusion table: | GBM | Healthy |
|---|---|---|
| Predicted as GBM | 10 | 0 |
| Predicted as Healthy | 0 | 10 |
| Predicted as unassigned | 0 | 0 |

TABLE 14
Statistical results of PLS-DA model performance
using serum samples for GBM diagnosis.

| | Calibration | Cross Validation | Prediction |
|---|---|---|---|
| R² | 0.998296 | 0.985529 | 0.998296 |
| Error | RMSEC: 0.0206396 | RMSECV: 0.0629593 | RMSEP: 0.0206396 |
| Sensitivity | 1.000 | 1.000 | 1.000 |
| Specificity | 1.000 | 1.000 | 1.000 |
| Classification Error | 0 | 0 | 0 |

TABLE 15
Cross validation confusion matrix for
using serum samples for GBM diagnosis.

| Class | TPR | FPR | TNR | FNR | Err |
|---|---|---|---|---|---|
| GBM | 1.00000 | 0.00000 | 1.00000 | 0.00000 | 0.00000 |
| Healthy | 1.00000 | 0.00000 | 1.00000 | 0.00000 | 0.00000 |

Actual Class

| Confusion table: | GBM | Healthy |
|---|---|---|
| Predicted as GBM | 10 | 0 |
| Predicted as Healthy | 0 | 10 |
| Predicted as unassigned | 0 | 0 |

### Example 13—Use of NK Cell Extracellular Vesicles for Distinguishing Between Astrocytoma, Oligodendroglioma, and Glioblastoma

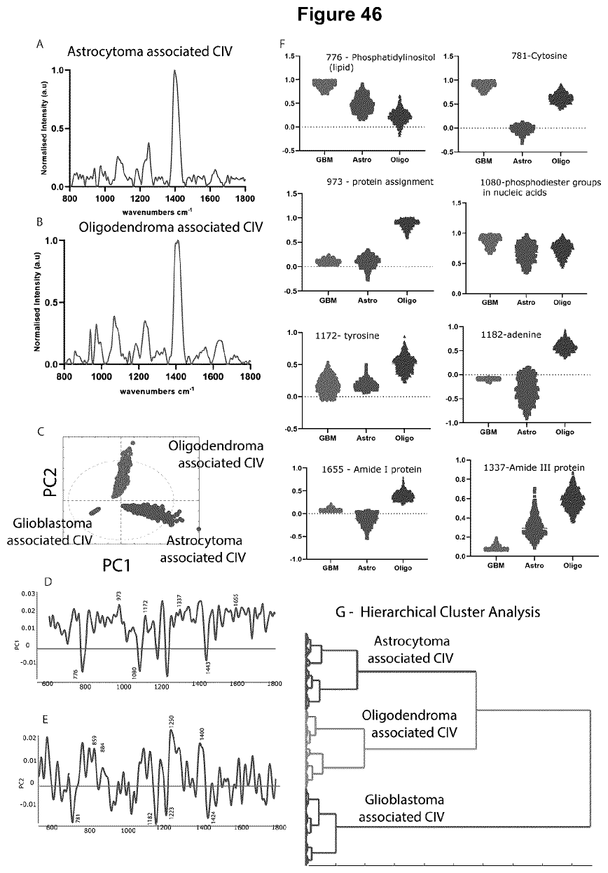

The term glioma encompassed astrocytoma, oligodendroglioma, and glioblastoma. While GBM is a tumor of high grade (grade IV), astrocytoma and oligodendroglioma are tumors of low grade (grade II). Since GBM tumor has been demonstrated to educate NK cells, whether low grade gliomas such as astrocytoma and oligodendroglioma are also capable of educating NK cells was assessed, and thereby determining whether this results in metabolic changes in the tumor-experienced NK cell that will be reflected in the vesicles of NK cells, resulting in CIVs with a distinct metabolic profile. For this purpose, tumor cells were isolated from a patient diagnosed with a grade II astrocytoma and grade II oligodendroglioma. The isolated tumor cells were cocultured with NK cells, and following interaction, the supernatant was collected and centrifuged (according to the manufacturer's protocol) to produce EVs, which were subsequently named as astrocytoma associated CIV and oligodendroglioma related CIV, respectively. FIG. 46A and FIG. 46B show the Raman spectra of each. These Raman spectra were then compared with GBM associated CIVs to determine if NK-CIV can distinguish between low grade and high-grade tumors.

FIG. 46C shows principal component analysis using first two principal components showing Raman spectra's ability to clearly distinguish between the low grade (astrocytoma vs oligodendroglioma) and between low grade and high grade (glioblastoma) tumors. To further identify the critical spectral data points in characterizing low-grade and high-grade tumors, the spectral data points from the PC1 and PC2 loadings of PCA were taken. Spectral data points from the PC1 loading, namely, 776, 973, 1080, 1172, 1337, 1443, and 1665 cm⁻¹ which contributed most to distinguishing low grade oligodendroglioma from high grade GBM (FIG. 46D), were chosen as the candidate biomarkers. Notably, the spectral data points linked with proteins (973, 1172, 1337, and 1655 cm⁻¹) had positive values, while the spectral data points related with lipids (776, 1080, and 1443 cm⁻¹) had negative values. The above analyses led to the selection of these seven spectral data points as potential differentiation markers to differentiate oligodendroglioma from GBM. Similarly, the spectral data points from the PC2 loading, namely, 884, 859, 1250, 1400 and 781, 1182, 1223, 1424 cm⁻¹, which contributed most to separating low grade astrocytoma from high grade GBM (FIG. 46E), were selected as the possible biomarkers to identify astrocytoma from GBM. The spectrum data points associated with proteins had positive values (884, 859, 1250, 1400 cm⁻¹), whereas the spectral data points associated with nucleic acids had negative values (781, 1182, 1223, 1424 cm⁻¹).

### Example 14—Detection of Hard to Detect Ovarian Cancer with the Extracellular Vesicles of Tumor Associated T Cells

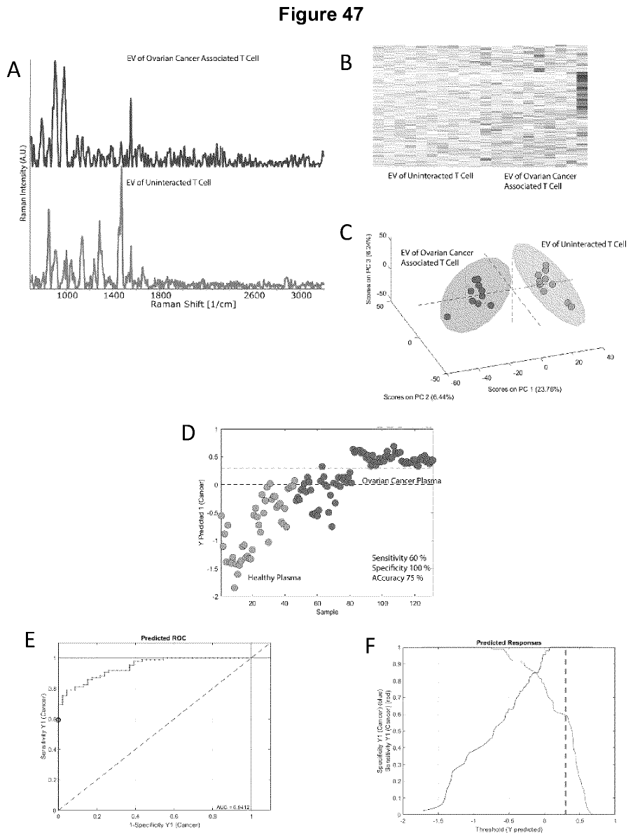

Raman spectral signatures were obtained of extracellular vesicles (EVs) from healthy T cells and T cells associated with a pre-clinical model of ovarian cancer (FIG. 47, A). Both a heatmap (FIG. 47, B) and principal component analysis (FIG. 47, C) demonstrate clear and discrete clustering of healthy vs ovarian cancer associated T cell EVs. Cross-validation of the pre-clinical model was undertaken using patient plasma (FIG. 47, D) and demonstrated that the model can classify a sample as ovarian cancer with 60% sensitivity and 100% specificity (FIG. 47, F).

### Example 15—Early Diagnosis of Brain Cancer with Tumor Associated T Cells

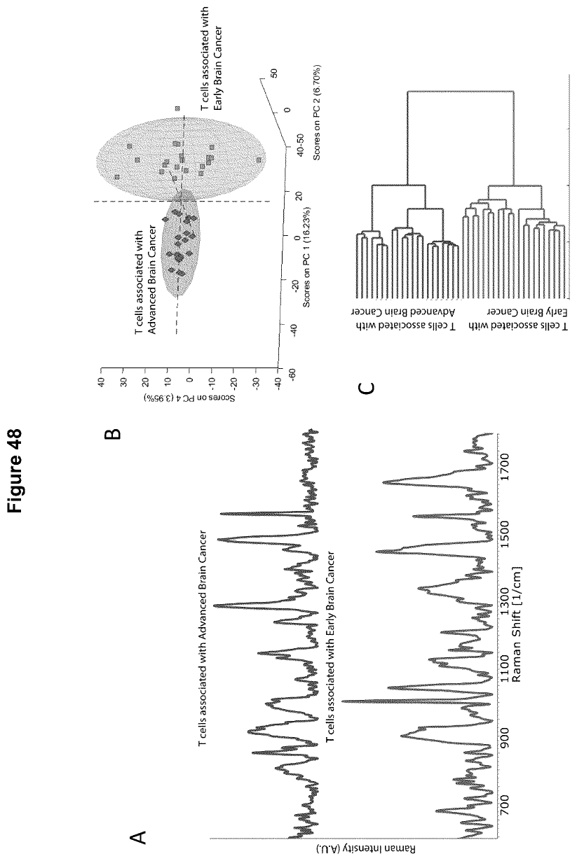

Raman spectral signatures were obtained from T cells associated with pre-clinical models of early brain cancer and advanced brain cancer (FIG. 48). These demonstrated distinct clusters for early vs advanced brain cancer in the pre-clinical model (FIG. 48, C).

### Example 16—Application Tumor Associated Immune Cells for Differentiating Benign and Malignant Cancer

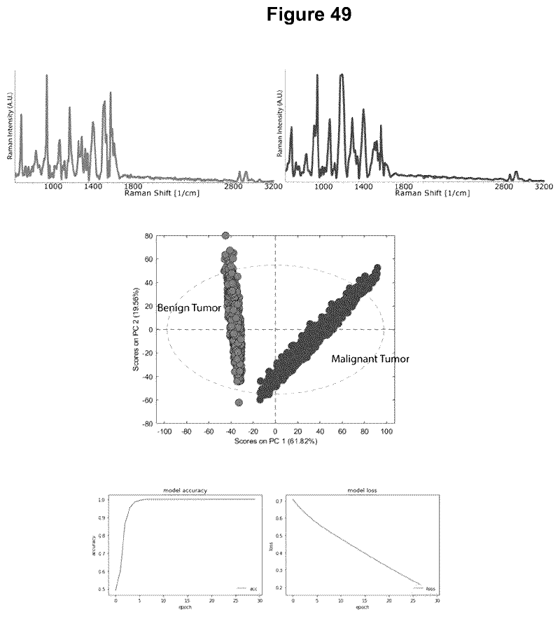

T cells play a critical role in recognizing tumor cells, including those that are from benign tumors. T cells, in particular, have been shown to infiltrate benign brain tumors like meningiomas and may play a role in controlling tumor growth. T cells also induce an immune response that can lead to the recognition and destruction of nearby tumor cells. T cell responds to benign tumors by recognizing the specific tumor-associated antigens and attracting other immune cells to the tumor site. By studying the T cell immune response to benign brain tumors, here in this study, meningiomas were distinguished from other malignant gliomas with a high level of accuracy as demonstrated in FIG. 49. The confusion matrix for the ANN prediction is in Table 16.

TABLE 16
Confusion matrix for ANN prediction
as a benign or malignant tumor.

| | Actually Benign | Actually Malignant |
|---|---|---|
| Predicted as Benign Tumor | 214 | 0 |
| Predicted as Malignant Tumor | 0 | 186 |

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### CITATIONS FOR REFERENCES REFERRED TO WITH IN-TEXT CITATIONS IN THE SPECIFICATION

Ames E. et al., “NK cells preferentially target tumor cells with a cancer stem cell phenotype,” The Journal of Immunology, vol. 195, no. 8, pp. 4010-4019, 2015.
Butler K. T., Davies D. W., Cartwright H., Isayev O., and Walsh A., “Machine learning for molecular and materials science,” Nature, vol. 559, no. 7715, pp. 547-555, 2018.
El-Said W. A., Kim T.-H., Kim H., and Choi J.-W., “Analysis of Intracellular State Based on Controlled 3D Nanostructures Mediated Surface Enhanced Raman Scattering,” PLOS ONE, vol. 6, no. 2, p. e15836, February 2011, doi: 10.1371/journal.pone.0015836.
Gentles, A. J. et al. The prognostic landscape of genes and infiltrating immune cells across human cancers. Nat. Med. 21, 938-945 (2015).
Guo S., Popp J., and Bocklitz T., “Chemometric analysis in Raman spectroscopy from experimental design to machine learning-based modeling,” Nature protocols, vol. 16, no. 12, pp. 5426-5459, 2021.
Himstedt R. et al., “Localized surface plasmon resonances of various nickel sulfide nanostructures and Au—Ni3S2 core-shell nanoparticles,” Chemistry of Materials, vol. 29, no. 17, pp. 7371-7377, 2017.
Ichimura, T. et al. Non-label immune cell state prediction using Raman spectroscopy. Sci. Rep. 6, 1-7 (2016).
Jiang S., Munker R., and Andreeff M., “Bcl-2 is expressed in human natural killer cells and is regulated by interleukin-2,” Nat. Immun. vol. 15, no. 6, pp. 312-7, 1997 1996.
Kornberg M. D., “The immunologic Warburg effect: Evidence and therapeutic opportunities in autoimmunity,” Wiley Interdiscip Rev Syst Biol Med, vol. 12, no. 5, pp. e1486-e1486, September 2020, doi: 10.1002/wsbm.1486.
Kresovich, J. K. et al. “Prediagnostic Immune Cell Profiles and Breast Cancer.” JAMA Netw. Open 3, 1-14 (2020).
López-Soto A., Gonzalez S., Smyth M. J., and Galluzzi L., “Control of metastasis by NK cells,” Cancer cell, vol. 32, no. 2, pp. 135-154, 2017.
Luna J. I., Grossenbacher S. K., Murphy W. J., and Canter R. J., “Natural Killer Cell Immunotherapy Targeting Cancer Stem Cells,” Expert opinion on biological therapy, vol. 17, no.3, p. 313, 2017.
Meza Ramirez C. A., Greenop M., Ashton L., and Rehman I. U., “Applications of machine learning in spectroscopy,” Applied Spectroscopy Reviews, vol. 56, no. 8-10, pp. 733-763, 2021.
Movasaghi, Z., Rehman, S. & Rehman, I. U. Raman spectroscopy of biological tissues. Appl. Spectrosc. Rev. 42, 493-541 (2007).
Myers, J. A. and Miller J. S., “Exploring the NK cell platform for cancer immunotherapy,” Nature reviews Clinical oncology, vol. 18, no. 2, pp. 85-100, 2021.
Pirzadeh Z., Pakizeh T., Miljkovic V., Langhammer C., and Dmitriev A., “Plasmon-interband coupling in nickel nanoantennas,” Acs Photonics, vol. 1, no. 3, pp. 158-162, 2014.
Ronald, J. A., Chuang, H. Y., Dragulescu-Andrasi, A., Horia, S. S. & Gambhira, S. S. Detecting cancers through tumor-activatable minicircles that lead to a detectable blood biomarker. Proc. Natl. Acad. Sci. U.S.A. 112, 3068-3073 (2015).
Salmon, H. & Donnadieu, E. Within tumors, interactions between T cells and tumor cells are impeded by the extracellular matrix. Oncoimmunology 1, 992 (2012).
Savas, P. et al. Clinical relevance of host immunity in breast cancer: from TILs to the clinic. Nat. Rev. Clin. Oncol. 13, 228-241 (2016).
Sheetz K. E. and Squier J., “Ultrafast optics: Imaging and manipulating biological systems,” Journal of Applied Physics, vol. 105, no. 5, p. 2, 2009.
Shen Y., Yue J., Xu W., and Xu S., “Recent progress of surface-enhanced Raman spectroscopy for subcellular compartment analysis,” Theranostics, vol. 11, no. 10, pp. 4872-4893, 2021, doi: 10.7150/thno.56409.
Shibue T. and Weinberg R. A., “EMT, CSCs, and drug resistance: the mechanistic link and clinical implications,” Nature reviews Clinical oncology, vol. 14, no. 10, pp. 611-629, 2017.
Tallerico R. et al., “Human NK cells selective targeting of colon cancer-initiating cells: a role for natural cytotoxicity receptors and MHC class I molecules,” The Journal of Immunology, vol. 190, no. 5, pp. 2381-2390, 2013.
Varn, F. S., Tafe, L. J., Amos, C. 1. & Cheng, C. Computational immune profiling in lung adenocarcinoma reveals reproducible prognostic associations with implications for immunotherapy. Oncoimmunology 7, 1-10 (2018).
Wellenstein, M. D. & de Visser, K. E. Cancer-Cell-Intrinsic Mechanisms Shaping the Tumor Immune Landscape. Immunity 48, 399-416 (2018).
Wong D. M., Bol'shakov A. A., and Russo R. E., “Laser induced breakdown spectroscopy,” in Encyclopedia of Spectroscopy and Spectrometry, 2010, pp. 1281-1287.
Xu Y. et al., “Killer immunoglobulin-like receptors/human leukocyte antigen class-1, a crucial immune pathway in cancer,” Annals of translational medicine, vol. 8, no. 5, 2020.
Zhao, W. et al. Deconvolution of cell type-specific drug responses in human tumor tissue with single-cell RNA-seq. Genome Med. 13, (2021).

### CITATIONS FOR REFERENCES REFERRED TO WITH NUMBERED CITATIONS IN THE SPECIFICATION

1. Waldman, A. D., Fritz, J. M. & Lenardo, M. J. A guide to cancer immunotherapy: from T cell basic science to clinical practice. Nat Rev Immunol 20, 651-668 (2020).
2. Ren, H., Cao, K. & Wang, M. A Correlation Between Differentiation Phenotypes of Infused T Cells and Anti-Cancer Immunotherapy. Frontiers in Immunology 12, (2021).
3. Andreatta, M. et al. Interpretation of T cell states from single-cell transcriptomics data using reference atlases. Nat Commun 12, 2965 (2021).
4. Whitaker, K. Earlier diagnosis: the importance of cancer symptoms. The Lancet Oncology 21, 6-8 (2020).
5. Reading, J., Foster, K., Joshi, K. & Chain, B. Tracking down tumor-specific T cells. Cancer Cell 40, 351-353 (2022).
6. Wang, Q. et al. Genetic associations of T cell cancer immune response-related genes with T cell phenotypes and clinical outcomes of early-stage lung cancer. J Immunother Cancer 8, e000336 (2020).
7. Guo, G. et al. Immune cell concentrations among the primary tumor microenvironment in colorectal cancer patients predicted by clinicopathologic characteristics and blood indexes. Journal for ImmunoTherapy of Cancer 7, 1-13 (2019).
8. Wu, S. Y., Fu, T., Jiang, Y. Z. & Shao, Z. M. Natural killer cells in cancer biology and therapy. Molecular Cancer 19, 1-26 (2020).
14. Nakamura, K. & Smyth, M. J. Immunoediting of cancer metastasis by NK cells. Nat Cancer 1, 670-671 (2020).
15. Mantovani, A., Allavena, P., Marchesi, F. & Garlanda, C. Macrophages as tools and targets in cancer therapy. Nat Rev Drug Discov 21, 799-820 (2022).
16. Cendrowicz, E., Sas, Z., Bremer, E. & Rygiel, T. P. The Role of Macrophages in Cancer Development and Therapy. Cancers (Basel) 13, 1946 (2021).
17. Kumari, N. & Choi, S. H. Tumor-associated macrophages in cancer: recent advancements in cancer nanoimmunotherapies. Journal of Experimental & Clinical Cancer Research 41, 68 (2022).
18. Poh, A. R. & Ernst, M. Targeting Macrophages in Cancer: From Bench to Bedside. Frontiers in Oncology 8, (2018).
19. Balá\hat{z}ová, K., Clevers, H. & Dost, A. F. The role of macrophages in non-small cell lung cancer and advancements in 3D co-cultures. eLife 12, e82998 (2023).
20. Condeelis, J. & Pollard, J. W. Macrophages: Obligate partners for tumor cell migration, invasion, and metastasis. Cell 124, 263-266 (2006).
21. Conway, E. M. et al. Macrophages, inflammation, and lung cancer. American Journal of Respiratory and Critical Care Medicine 193, 116-130 (2016).
24. Duan, Z. & Luo, Y. Targeting macrophages in cancer immunotherapy. Sig Transduct Target Ther 6, 1-21 (2021).
25. Dendritic cells in cancer immunology and immunotherapy|Nature Reviews Immunology. https://www.nature.com/articles/s41577-019-0210-z.
26. Del Prete, A. et al. Functional Role of Dendritic Cell Subsets in Cancer Progression and Clinical Implications. Int J Mol Sci 21, 3930 (2020).
27. Veglia, F. & Gabrilovich, D. I. Dendritic cells in cancer: the role revisited. Curr Opin Immunol 45, 43-51 (2017).
28. Lee, Y. S. & Radford, K. J. The role of dendritic cells in cancer. Int Rev Cell Mol Biol 348, 123-178 (2019).
29. Zanna, M. Y. et al. Review of Dendritic Cells, Their Role in Clinical Immunology, and Distribution in Various Animal Species. Int J Mol Sci 22, 8044 (2021).
30. Wang, H. et al. Metabolic labeling and targeted modulation of dendritic cells. Nature Materials (2020) doi:10.1038/s41563-020-0680-1.
31. Engelhard, V. et al. B cells and cancer. Cancer Cell 39, 1293-1296 (2021).
32. Shang, J., Zha, H. & Sun, Y. Phenotypes, Functions, and Clinical Relevance of Regulatory B Cells in Cancer. Front Immunol 11, 582657 (2020).
35. Hao, D. et al. The Single-Cell Immunogenomic Landscape of B and Plasma Cells in Early-Stage Lung Adenocarcinoma. Cancer Discovery 12, 2626-2645 (2022).
36. Wang, S. et al. Tumor-infiltrating B cells: their role and application in anti-tumor immunity in lung cancer. Cell Mol Immunol 16, 6-18 (2019).
37. Monzon, F. A., Medeiros, F., Lyons-Weiler, M. & Henner, W. D. Identification of tissue of origin in carcinoma of unknown primary with a microarray-based gene expression test. Diagnostic Pathology 5, 3 (2010).
38. Nguyen, L., Van Hoeck, A. & Cuppen, E. Machine learning-based tissue of origin classification for cancer of unknown primary diagnostics using genome-wide mutation features. Nat Commun 13, 4013 (2022).
39. Miao, Y. et al. Identifying cancer tissue-of-origin by a novel machine learning method based on expression quantitative trait loci. Front Oncol 12, 946552 (2022).
40. Visvader, J. E. Cells of origin in cancer. Nature 469, 314-322 (2011).
41. Xia, D. et al. Minimalist approaches to cancer tissue-of-origin classification by DNA methylation. Modern Pathology 33, 1874-1888 (2020).
42. Murata, M. Inflammation and cancer. Environmental Health and Preventive Medicine 23, 50 (2018).
43. Treffers, L. W., Hiemstra, I. H., Kuijpers, T. W., van den Berg, T. K. & Matlung, H. L. Neutrophils in cancer. Immunol Rev 273, 312-328 (2016).
44. Mishalian, I., Granot, Z. & Fridlender, Z. G. The diversity of circulating neutrophils in cancer. Immunobiology 222, 82-88 (2017).
45. Uribe-Querol, E. & Rosales, C. Neutrophils in Cancer: Two Sides of the Same Coin. J Immunol Res 2015, 983698 (2015).
46. Fridlender, Z. G. et al. Polarization of Tumor-Associated Neutrophil Phenotype by TGF-β: “N1” versus “N2” TAN. Cancer Cell 16, 183-194 (2009).
47. Xiong, S., Dong, L. & Cheng, L. Neutrophils in cancer carcinogenesis and metastasis. J Hematol Oncol 14, 173 (2021).
48. Dissemond, J. et al. Activated neutrophils exert antitumor activity against human melanoma cells: reactive oxygen species-induced mechanisms and their modulation by granulocyte-macrophage-colony-stimulating factor. J Invest Dermatol 121, 936-938 (2003).
49. Wang, T. et al. Tumour-activated neutrophils in gastric cancer foster immune suppression and disease progression through GM-CSF-PD-L1 pathway. Gut 66, 1900-1911 (2017).
50. Lei, X. et al. Immune cells within the tumor microenvironment: Biological functions and roles in cancer immunotherapy. Cancer Letters 470, 126-133 (2020).
51. Li, X. et al. Immune Cell Infiltration Landscape of Ovarian Cancer to Identify Prognosis and Immunotherapy-Related Genes to Aid Immunotherapy. Frontiers in Cell and Developmental Biology 9, (2021).
52. Gentles, A. J. et al. The prognostic landscape of genes and infiltrating immune cells across human cancers. Nature medicine 21, 938-945 (2015).
53. Kumar, S. & Dikshit, M. Metabolic Insight of Neutrophils in Health and Disease. Front Immunol 10, 2099 (2019).
54. Li, Y. et al. The regulatory roles of neutrophils in adaptive immunity. Cell Communication and Signaling 17, 147 (2019).
55. Zhao, H. et al. Inflammation and tumor progression: signaling pathways and targeted intervention. Sig Transduct Target Ther 6, 1-46 (2021).
56. Nicolas-Avila, J. A., Adrover, J. M. & Hidalgo, A. Neutrophils in homeostasis, immunity, and cancer. Immunity 46, 15-28 (2017).
57. Ramoji, A. et al. Toward a spectroscopic hemogram: Raman spectroscopic differentiation of the two most abundant leukocytes from peripheral blood. Analytical chemistry 84, 5335-5342 (2012).
58. Pully, V., Lenferink, A. T. & Otto, C. Time-lapse Raman imaging of single live lymphocytes. Journal of Raman spectroscopy 42, 167-173 (2011).
59. Sijtsema, N., Otto, C., Segers-Nolten, G. M., Verhoeven, A. & Greve, J. Resonance Raman microspectroscopy of myeloperoxidase and cytochrome b558 in human neutrophilic granulocytes. Biophysical journal 74, 3250-3255 (1998).
60. Puppels, G., Garritsen, H., Kummer, J. & Greve, J. Carotenoids located in human lymphocyte subpopulations and natural killer cells by Raman microspectroscopy. Cytometry: The Journal of the International Society for Analytical Cytology 14, 251-256 (1993).
61. van Manen, H.-J., Kraan, Y. M., Roos, D. & Otto, C. Intracellular chemical imaging of heme-containing enzymes involved in innate immunity using resonance Raman microscopy. The Journal of Physical Chemistry B 108, 18762-18771 (2004).
62. Baci, D. et al. The Ovarian Cancer Tumor Immune Microenvironment (TIME) as Target for Therapy: A Focus on Innate Immunity Cells as Therapeutic Effectors. Int J Mol Sci 21, 3125 (2020).
63. Yajuk, O. et al. The PD-L1/PD-1 Axis Blocks Neutrophil Cytotoxicity in Cancer. Cells 10, 1510 (2021).
64. Zhang, S. et al. Berberine Maintains the Neutrophil N1 Phenotype to Reverse Cancer Cell Resistance to Doxorubicin. Frontiers in Pharmacology 10, (2020).
65. Stein, R. G. et al. Cognate Nonlytic Interactions between CD8+ T Cells and Breast Cancer Cells Induce Cancer Stem Cell-like Properties. Cancer Research 79, 1507-1519 (2019).
66. Surface-Enhanced Raman Spectroscopy of DNA|Journal of the American Chemical Society. https://pubs.acs.org/doi/10.1021/ja800023j.
67. De Gelder, J., De Gussem, K., Vandenabeele, P. & Moens, L. Reference database of Raman spectra of biological molecules. Journal of Raman Spectroscopy 38, 1133-1147 (2007).
68. Ghebeh, H. et al. FOXP3+ Tregs and B7-H1+/PD-1+T lymphocytes co-infiltrate the tumor tissues of high-risk breast cancer patients: Implication for immunotherapy. BMC Cancer 8, 57 (2008).
69. Bates, G. J. et al. Quantification of regulatory T cells enables the identification of high-risk breast cancer patients and those at risk of late relapse. J Clin Oncol 24, 5373-5380 (2006).
70. Abo-Elenein, A., Elgohary, S. E. & Hashish, A. Significance of Immunoregulatory T Cells in Different Stages of Breast Cancer Patients. 8.
71. Leong P P, Mohammad R, Ibrahim N, Ithnin H, Abdullah M, Davis W C, Seow H F(2006). Phenotyping of lymphocytes expressing regulatory and effector markers in infiltrating ductal carcinoma of the breast. Immnol Lett. 102:229-36.—Google Search. https://www.google.com/search?q=Leong+PP %2C+Mohammad+R %2C+Ibrahim+N %2C+Ithnin+H %2C+Abdullah+M %2C+Davis+WC %2C+Seow+HF(2006).+Phenotyping+of+lymphocytes+expressing+regulatory+and+effector+markers+in+infiltrating+ductal+carcinoma+of+the+ breast.+Immnol+Lett.+102%3A229% E2%80%9336.&rlz=1C1CHBF_enCA899CA899&oq=Leong+PP %2C+Mohammad+R %2C+Ibrahim+N %2C+Ithnin+H %2C+Abdullah+M %2C+Davis+WC %2C+Seow+HF(2006).+Phenotyping+of+lymphocytes+expressing+regulatory+ and+effector+markers+in+infiltrating+ductal+carcinoma+of+the+ breast.+Immnol+Lett.+102%3A229% E2%80%9336.&aqs=chrome . . . 69i57.284j0j7&sourceid=chrome&ie=UTF-8.
72. Wang, Y., Wei, Z., Zhang, Y. & Chen, Y. Direct detection of DNA using 3D surface enhanced Raman scattering hotspot matrix. ELECTROPHORESIS 40, 2104-2111 (2019).
73. Barhoumi, A., Zhang, D., Tam, F. & Halas, N. J. Surface-Enhanced Raman Spectroscopy of DNA. ACS Publications http://pubs.acs.org/doi/full/10.1021/ja800023j (2008) doi:10.1021/ja800023j.
74. Zhou, J. & Zhong, Y. Breast cancer immunotherapy. Cell Mol Immunol 1, 247-255 (2004).
75. Ali, H. R., Chlon, L., Pharoah, P. D. P., Markowetz, F. & Caldas, C. Patterns of Immune Infiltration in Breast Cancer and Their Clinical Implications: A Gene-Expression-Based Retrospective Study. PLOS Medicine 13, e1002194 (2016).
76. Wang, S. et al. Clinical significance and immunogenomic landscape analyses of the immune cell signature based prognostic model for patients with breast cancer. Brief Bioinform 22, bbaa311 (2021).
77. Movasaghi, Z., Rehman, S. & Rehman, I. U. Raman spectroscopy of biological tissues. Applied Spectroscopy Reviews 42, 493-541 (2007).
78. Xing, X., Du, S. & Wang, K. Robust Hessian Locally Linear Embedding Techniques for High-Dimensional Data. Algorithms 9, 36 (2016).
79. Rivenbark, A. G., O'Connor, S. M. & Coleman, W. B. Molecular and Cellular Heterogeneity in Breast Cancer. Am J Pathol 183, 1113-1124 (2013).
80. Godoy-Ortiz, A., Alba-Bernal, A., Pascual, J., Comino-Mendez, I. & Alba, E. Unveiling the Potential of Liquid Biopsy in HER2-Positive Breast Cancer Management. Cancers 14, 587 (2022).
81. Lopes, C. et al. HER2 Expression in Circulating Tumour Cells Isolated from Metastatic Breast Cancer Patients Using a Size-Based Microfluidic Device. Cancers 13, 4446 (2021).
82. Pupa, S. M. et al. HER2 Signaling and Breast Cancer Stem Cells: The Bridge behind HER2-Positive Breast Cancer Aggressiveness and Therapy Refractoriness. Cancers (Basel) 13, 4778 (2021).
83. Li, X. et al. Uncovering the Subtype-Specific Molecular Characteristics of Breast Cancer by Multiomics Analysis of Prognosis-Associated Genes, Driver Genes, Signaling Pathways, and Immune Activity. Front Cell Dev Biol 9, 689028 (2021).
84. Fan, L., Ma, L.-X., Zhou, P. & Shao, Z.-M. Atlas of immune cell infiltration in breast cancer-high M2 macrophage and low native B cell proportions are associated with poor survival. Annals of Breast Surgery 6, (2022).
88. Croker, A. k & Allan, A. I. Cancer stem cells: implications for the progression and treatment of metastatic disease. Journal of Cellular and Molecular Medicine 12, 374-390 (2008).
89. Sheridan, C. et al. CD44+/CD24-Breast cancer cells exhibit enhanced invase properties: An early step necessary for metastasis. Breast Cancer Research 8, 1-13 (2006).
90. Turner, N. H. & Di Leo, A. HER2 discordance between primary and metastatic breast cancer: Assessing the clinical impact. Cancer Treatment Reviews 39, 947-957 (2013).
91. Zimmer, A. S., Van Swearingen, A. E. D. & Anders, C. K. HER2-positive breast cancer brain metastasis: A new and exciting landscape. Cancer Rep (Hoboken) 5, e1274 (2022).
92. Sammons, S., Van Swearingen, A. E. D., Chung, C. & Anders, C. K. Advances in the management of breast cancer brain metastases. Neuro-Oncology Advances 3, v63-v74 (2021).
93. Stanton, S. E. & Disis, M. L. Clinical significance of tumor-infiltrating lymphocytes in breast cancer. J Immunother Cancer 4, 59 (2016).
94. Lin, D. et al. Label-free liquid biopsy based on blood circulating DNA detection using SERS-based nanotechnology for nasopharyngeal cancer screening. Nanomedicine: Nanotechnology, Biology, and Medicine 22, 102100 (2019).
95. Papachristou, N. et al. Network Analysis of the Multidimensional Symptom Experience of Oncology. Sci Rep 9, 2258 (2019).
96. Bell, S. E. J. & Sirimuthu, N. M. S. Surface-Enhanced Raman Spectroscopy (SERS) for Sub-Micromolar Detection of DNA/RNA Mononucleotides. Journal of the American Chemical Society 128, 15580-15581 (2006).
97. Ganesh, S., Venkatakrishnan, K. & Tan, B. Quantum scale organic semiconductors for SERS detection of DNA methylation and gene expression. Nature Communications 11, (2020).
98. Martinez-Sáez, O. & Prat, A. Current and Future Management of HER2-Positive Metastatic Breast Cancer. JCO Oncology Practice 17, 594-604 (2021).
99. Fabi, A. et al. HER2 Protein and Gene Variation between Primary and Metastatic Breast Cancer: Significance and Impact on Patient Care. Clinical Cancer Research 17, 2055-2064 (2011).
100. Liu, M. et al. Classification using hierarchical clustering of tumor-infiltrating immune cells identifies poor prognostic ovarian cancers with high levels of COX expression. Mod Pathol 22, 373-384 (2009).
101. Wang, T. et al. Immunogenomic Landscape in Breast Cancer Reveals Immunotherapeutically Relevant Gene Signatures. Front Immunol 13, 805184 (2022).
102. Spranger, S. Mechanisms of tumor escape in the context of the T-cell-inflamed and the non-T-cell-inflamed tumor microenvironment. International Immunology 28, 383-391 (2016).
103. Horton, B. L. et al. Lack of CD8+ T cell effector differentiation during priming mediates checkpoint blockade resistance in non-small cell lung cancer. Science Immunology 6, eabi8800.
104. Yang, J., Xu, J., E, Y. & Sun, T. Predictive and prognostic value of circulating blood lymphocyte subsets in metastatic breast cancer. Cancer Med 8, 492-500 (2019).
105. DeNardo, D. G. et al. CD4+ T Cells Regulate Pulmonary Metastasis of Mammary Carcinomas by Enhancing Protumor Properties of Macrophages. Cancer Cell 16, 91-102 (2009).
106. Karavitis, J. et al. Regulation of COX2 Expression in Mouse Mammary Tumor Cells Controls Bone Metastasis and PGE2-Induction of Regulatory T Cell Migration. PLOS ONE 7, e46342 (2012).
107. Janssen, L. M. E., Ramsay, E. E., Logsdon, C. D. & Overwijk, W. W. The immune system in cancer metastasis: friend or foe? Journal for ImmunoTherapy of Cancer 5, 79 (2017).
108. Olkhanud, P. B. et al. Breast Cancer Lung Metastasis Requires Expression of Chemokine Receptor CCR4 and Regulatory T Cells. Cancer Research 69, 5996-6004 (2009).
109. de Kruijf, E. M. et al. NKG2D ligand tumor expression and association with clinical outcome in early breast cancer patients: an observational study. BMC Cancer 12, 24 (2012).
110. Coffelt, S. B. et al. IL-17-producing γδ T cells and neutrophils conspire to promote breast cancer metastasis. Nature 522, 345-348 (2015).
111. Kuhn, A., Thu, D., Waldvogel, H. J., Faull, R. L. M. & Luthi-Carter, R. Population-specific expression analysis (PSEA) reveals molecular changes in diseased brain. Nat Methods 8, 945-947 (2011).
112. Kang, K. et al. CDSeq: A novel complete deconvolution method for dissecting heterogeneous samples using gene expression data. PLOS Computational Biology 15, e1007510 (2019).
113. Hu, Y. et al. False-positive plasma genotyping due to clonal hematopoiesis. Clinical Cancer Research 24, 4437-4443 (2018).
114. Freudenberg, J. A. et al. The role of HER2 in early breast cancer metastasis and the origins of resistance to HER2-targeted therapies. Exp Mol Pathol 87, 1-11 (2009).
115. Cardoso, F. et al. 5th ESO-ESMO international consensus guidelines for advanced breast cancer (ABC 5). Annals of Oncology 31, 1623-1649 (2020).

1. A method of determining one or more cancer characteristics for a subject, the method comprising:
a. measuring a value of one or more cancer traits of at least one tumor associated immune cell (TAIC) or TAIC-derived biomarker in a fluid sample obtained from the subject to obtain a test profile;
b. comparing the test profile to one or more reference profiles;
c. wherein similarity or a differential in the test profile to the one or more reference profiles is indicative of one or more cancer characteristics; and optionally,
d. based on the one or more cancer characteristics, providing a cancer assessment of the subject.

2. The method of claim 1, wherein the cancer characteristic is a presence or absence of cancer, a cancer type, a cancer stage, a cancer grade, a primary cancer, a metastatic cancer, cancer potential for metastasis, a molecular phenotype of the cancer, a benign cancer, a malignant cancer, the tissue of origin of the cancer, a metastatic location of the cancer or a combination thereof.

3. The method of claim 1, wherein the at least one tumor associated immune cell comprises a T cell, a NK cell, a macrophage, a neutrophil, a dendritic cell or a B cell, or a combination thereof.

4. The method of claim 1, wherein the one or more TAIC-derived biomarkers comprise metabolic changes, activation status or a surface phenotype of the at least one tumor associated immune cell.

5. The method of claim 1, wherein the one or more TAIC-derived biomarkers comprise one or more extracellular vesicles from the at least one tumor associated immune cell.

6. The method of claim 1, wherein the one or more TAIC-derived biomarkers comprise cell-free nucleic acid of the at least one tumor associated immune cell.

7. The method of claim 6, wherein the cell-free nucleic acid is cell-free DNA, miRNA, lncRNA, mRNA or histone-associated DNA, optionally wherein the one or more TAIC-derived biomarkers comprise the structure and molecular composition of the cell-free DNA.

8. The method of claim 7, wherein the cell-free DNA is molecularly modified by one or more of methylation, oxidation, phosphorylation and acetylation.

9. (canceled)

10. The method of claim 1, wherein the one or more TAIC-derived biomarkers comprise DNA methylation status of the at least one tumor associated immune cell.

11. The method of claim 1, wherein the one or more cancer traits is measured by Raman spectroscopy.

12. The method of claim 1, wherein the one or more cancer traits is measured by DNA Next gen sequencing, PCR, repertoire sequencing, immunosequencing, single cell RNA sequencing, DNA methylation sequencing, unique molecular identifier (UMI), optical microscopy techniques, immunohistochemistry, super resolution microscopy, flow cytometry, or mass cytometry.

13. The method of claim 1, wherein the reference profile is derived from one or more reference TAICs, optionally wherein the reference TAIC is a T cell, a NK cell, a macrophage, a neutrophil, a dendritic cell, or a B cell, or a combination thereof.

14. (canceled)

15. The method of claim 13, wherein the reference TAIC is:
i) a cancer cell associated T cell, a cancer stem cell associated T cell, or a tumor associated stem cell like T cell,
(ii) a cancer cell associated NK cell, a cancer stem cell associated NK cell, or a tumor associated stem cell like NK cell,
(iii) is cancer cell associated macrophage, a cancer stem cell associated macrophage, or a tumor associated stem cell like macrophage,
(iv) is a cancer cell associated neutrophil, a cancer stem cell associated neutrophil, or a tumor associated stem cell like neutrophil,
(v) is a cancer cell associated dendritic cell, a cancer stem cell associated dendritic cell, or a tumor associated stem cell like dendritic cell or
(vi) is a cancer cell associated B cell, a cancer stem cell associated B cell, or a tumor associated stem cell like B cell.

16.-20. (canceled)

21. The method of claim 1, wherein the fluid is blood plasma, serum, urine, stool, mucus or cerebrospinal fluid.

22. The method of claim 1, wherein providing the cancer assessment comprises providing a cancer type, a cancer location, a stage of the cancer, a grade of the cancer, a metastatic potential of the cancer, or a cancer therapy efficacy.

23. The method of claim 1, wherein providing the cancer assessment comprises providing a prognosis for the patient, providing an early cancer diagnosis, determining whether a tumor is benign or malignant, determining whether a tumor is primary or metastatic, determining whether a tumor has potential for metastasis, determining a progression of the cancer, determining a nodal metastasis of the cancer, determining a clinical metastasis of the cancer, predicting patient survival, providing a prognosis for the patient, determining a presence of an aggressive brain cancer, providing a location of the tumor, monitoring cancer recurrence during or after therapy, and/or determining a presence of minimal residual disease.

24. The method of claim 23, wherein when the tumor is benign, determining whether the tumor has potential for malignancy.

25. The method of claim 1, wherein the cancer is brain cancer, optionally glioblastoma, astrocytoma, or oligodendroglioma.

26. The method of claim 1, wherein the cancer is ovarian cancer, brain cancer, bladder cancer, breast cancer, colon cancer, esophageal cancer, gastric cancer, hepatic cancer, intestinal cancer, lung cancer, head and neck cancer, rectal cancer, prostrate cancer, pancreatic cancer, thyroid cancer, cervical cancer, melanoma, nasopharyngeal cancer, testicular cancer, or uterine cancer.

27. (canceled)

28. A method of determining one or more cancer characteristics for a subject, the method comprising:
a. isolating a volume of a fluid from a fluid sample of the patient, the volume of fluid including at least one tumor associated immune cell (TAIC) or TAIC-derived biomarker;
b. adding at least a portion of the volume of fluid to a nanosensor, the nanosensor comprising nanoparticles configured to capture the at least one TAIC) or TAIC-derived biomarker and amplify signals emitted by the at least one TAIC or TAIC-derived biomarker during Raman spectroscopy;
c. performing Raman spectroscopy on the volume of fluid on the nanosensor to produce a sample Raman spectrum, the sample Raman spectrum having amplified signals indicating the presence of the at least one TAIC or TAIC-derived biomarker on the nanosensor;
d. processing the sample Raman spectrum using data from template Raman spectra having cancer characteristics to detect whether the sample comprises one or more of the cancer characteristics; and optionally
e. based on the detected one or more cancer characteristics, providing a cancer assessment of the subject.

29.-47. (canceled)

48. A method of generating a reference tumor associated immune cell (TAIC) for use in cancer assessment, the method comprising coculturing one or more naïve immune cells with a tumor and/or cancer cell line until the one or more reference tumor associated immune cell (TAICs) expresses one or more cancer traits.

49.-50. (canceled)
